(19) United States
(12) Patent Application Publication
(10) Pub. No.: US 2009/0275570 A1
(43) Pub. Date: Nov. 5, 2009

(54) SUBSTITUTED HETEROCYCLES AND THEIR USE AS CHK1, PDK1 AND PAK INHIBITORS

(75) Inventors: Kevin Daly, Waltham, MA (US); Nicola Heron, Macclesfield (GB); Alexander Hird, Waltham, MA (US); Stephanos Ioannidis, Waltham, MA (US); James Janetka, Waltham, MA (US); Paul Lyne, Waltham, MA (US); Jaime Scott, Macclesfield (GB); Dorin Toader, Waltham, MA (US); Melissa Vasbinder, Waltham, MA (US); Dingwei Yu, Waltham, MA (US); Yan Yu, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 11/910,358

(22) PCT Filed: Apr. 5, 2006
(86) PCT No.: PCT/GB06/01242

§ 371 Date: Oct. 1, 2007

Related U.S. Application Data
(60) Provisional application No. 60/668,779, filed on Apr. 6, 2005.
(60) Provisional application No. 60/738,866, filed on Nov. 21, 2005.

(51) Int. Cl. A61K 31/538; C07D 495/04; A61K 31/4365; A61K 31/5377; A61P 35/00; A61K 31/454; C07D 401/12

(52) U.S. Cl. 514/230.5; 546/114; 514/301; 514/233.8; 544/127; 544/105; 514/323; 546/201

(57) ABSTRACT

The invention relates to novel compounds of Formula (I) and to their pharmaceutical compositions and to their methods of use. These novel compounds possess CHK1 kinase inhibitory activity, PDK1 inhibitory activity and Pak kinase inhibitory activity and are accordingly useful in the treatment and/or prophylaxis of cancer.

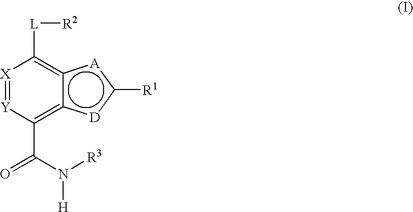

### FIELD OF THE INVENTION

The present invention relates to novel substituted heterocycles, their pharmaceutical compositions and methods of use. In addition, the present invention relates to therapeutic methods for the treatment and prevention of cancers.

### BACKGROUND OF THE INVENTION

Chemotherapy and radiation exposure are currently the major options for the treatment of cancer, but the utility of both these approaches is severely limited by drastic adverse effects on normal tissue, and the frequent development of tumor cell resistance. It is therefore highly desirable to improve the efficacy of such treatments in a way that does not increase the toxicity associated with them. One way to achieve this is by the use of specific sensitizing agents such as those described herein.

An individual cell replicates by making an exact copy of its chromosomes, and then segregating these into separate cells. This cycle of DNA replication, chromosome separation and division is regulated by mechanisms within the cell that maintain the order of the steps and ensure that each step is precisely carried out. Key to these processes are the cell cycle checkpoints (Hartwell et al., Science, Nov. 3, 1989, 246(4930):629-34) where cells may arrest to ensure DNA repair mechanisms have time to operate prior to continuing through the cycle into mitosis. There are two such checkpoints in the cell cycle—the G/S checkpoint that is regulated by p53 and the G2/M checkpoint that is monitored by the Ser/Thr kinase checkpoint kinase 1 (CHK1). In addition, Chk1 has also been recently identified to be important in the S phase checkpoint (Zhao et al. PNAS, Nov. 12, 2002, 99(23): 14795-14800; Sorsensen et al., Cancer Cell, March 2003, vol 3:247-258 and Senggupta et al., Journal of Cell Biology, vol 166, 6, 801-813).

As the cell cycle arrest induced by these checkpoints is a crucial mechanism by which cells can overcome the damage resulting from radio- or chemotherapy, their abrogation by novel agents should increase the sensitivity of tumor cells to DNA damaging therapies. Additionally, the tumor specific abrogation of the G1/S checkpoint by p53 mutations in the majority of tumors can be exploited to provide tumor selective agents. One approach to the design of chemosensitizers or radiosensitizers that abrogate the G2/M checkpoint is to develop inhibitors of the key G2/M regulatory kinase CHK1, and this approach has been shown to work in a number of proof of concept studies. (Koniaras et al., Oncogene, 2001, 20:7453; Luo et al., Neoplasia, 2001, 3:411; Busby et al., Cancer Res., 2000, 60:2108; Jackson et al., Cancer Res., 2000, 60:566).

Certain kinases that belong to the serine/threonine kinase family and are located intracellularly and are involved in the transmission of biochemical signals such as those that influence tumour cell growth. Such serine/threonine kinase signalling pathways include the Raf-MEK-ERK cascade and those downstream of PI3K such as PDK-1, AKT and mTOR (Blume-Jensen and Hunter, Nature, 2001, 411, 355). These serine/threonine kinase pathways have also been show to regulate, and be regulated by, other serine/threonine kinases that also regulate tumour growth and invasion. One such family of kinases is the p21-activated protein kinase (Pak) family of intracellular serine/threonine kinases.

The Pak family of kinases act as downstream effectors of the small p21 Rho GTPases, Rac and Cdc42 (Bokoch, Annual Review of Biochemistry, 2003, 72, 741-781). Six human Pak kinases have been identified which fall into two subfamilies. The first subfamily (Group I) consists of Pak1 (Pakoc), Pak2 (Pakγ, hPak65) and Pak3 (Pak13). The other subfamily (Group II) includes Pak4, Pak5 and Pak6. Group I family Paks share 93% identity in their kinase domains whereas the kinase domains of Group II Paks are more diverged displaying 54% identity with Group I kinase domains. Group 1 Pak kinases can be activated by a variety of GTPase-dependent and -independent mechanisms. Group 1 Pak kinases interact with activated (GTP-bound) p21 (Rac/Cdc42), inhibiting the GTPase activity of p21 and leading to kinase autophosphorylation and activation. Guanine nucleotide exchange factors (GEFs) and GTPase-activating proteins (GAPs), which regulate the GTP-GDP bound states of the Rho family of GTPases, are important determinants of downstream signalling activated by Pak.

The Pak family of kinases have been implicated in the regulation of cell survival, transformation, proliferation and cell motility (Bokoch, Annual Review of Biochemistry, 2003, 72, 741-781; Kumar and Hung, Cancer Research, 2005, 65, 2511-2515). Pak1 signals downstream of the Ras pathway and activation of Pak has been shown to have a role in cellular transformation. As in simpler eukaryotes, Paks in mammalian cells regulate MAPK signalling pathways, for example, Pak1 phosphorylates both Raf1 and Mek1. Paks play an important role in growth factor signalling, leading to cytoskeletal reorganisation that influences growth factor-mediated migration and invasion. Pak1 activation also promotes cell survival by inactivating Bad, suggesting that Pak1 may be involved in cancer cell survival and progression

There is now emerging data that the Pak family of kinases contribute to tumourigenesis in a wide range of human cancers, either directly or indirectly (Vadlamudi and Kumar, 2003, Cancer and Metastasis Reviews, 2003, 22, 385-393; Kumar and Hung, Cancer Research, 2005, 65, 2511-2515). For example, Pak1 gene amplification and a corresponding up-regulation of Pak1 protein has been reported in ovarian breast tumours (Schraml et al., American Journal of Pathology, 2003, 163, 985-992). Pak1 expression has been reported to increase with progression of colorectal carcinoma to metastasis (Carter et al., Clinical Cancer Research, 2004, 10, 3448-3456). Furthermore, Pak4 gene amplification and mutation has been identified in colorectal kinases (Parsons et al., Nature, 2005, 436, 792). Emerging data suggests that Pak1 is involved in breast cancer progression. For example, expression of a constitutively active Pak1 transgene in mouse mammary glands induces hyerplasia in the mammary epithelium (Wang et al., The EMBO Journal, 2002, 21, 5437-5447). Finally, the regulation of Pak activity by Rac/Cdc42 and Guanine Exchange Factors (GEFs) may also participate in the hyperactivation of Pak signalling cascades in cancer. For example, emerging data around a key role for the GEF Vav1 in pancreatic cancer tumourigenesis has revealed a potential opportunity to target the Rac-Pak signalling pathway in the treatment of pancreatic tumours (Fernandez-Zapico et al., Cancer Cell, 2005, 7, 39-49).

These findings suggest that pharmacological inhibitors of Pak should be of therapeutic value for treatment of the various forms of the disease of cancer.

There is also evidence that Pak plays a role in regulating neural outgrowth and normal brain development (Hofmann et al., Journal of Cell Science, 2004, 117, 4343-4354; Nikolic, The International Journal of Biochemistry, 2002, 34, 731-745). Pak inhibitors may be useful in the treatment of neural degenerative diseases and diseases associated with defective neural regeneration. Furthermore, Pak inhibitors may also have potential application in the treatment of a joint disease or of joint pain.

The phosphatidylinositol 3′OH kinase (PI3K) pathway is known to be intrinsically involved in regulating cell survival and apoptosis (Yao and Cooper, Oncogene, 1996, 13, 343-351; Franke et al, Oncogene, 2003, 22, 8983-8998) As part of this pathway phosphoinositide dependent protein kinase-1 (PDK1) and Akt play pivotal roles in signal transduction (Vanhaesebroeck and Alessi, Biochem. J., 2000, 346, 561-576). Activation of PI3K leads to production of phosphatidylinositol (3,4,5) triphosphate, which binds to the pleckstrin homology regions of PDK1 and Akt to effect membrane association and activation of Akt. Gene mutations of PI3K pathway kinases such as PI3K, Akt, mTOR have been closely associated with several human cancers including those of the colon, breast and prostate (Philp et al, Cancer Res., 2001, 61, 7426-7429; Bellacosa et al, Int. J. Cancer, 1995, 64, 280-285). Perturbation of this pathway by mutation or deletion of PTEN, a lipid phosphatase that reduces cellular PIP3, is associated with a variety of human tumours including breast, prostate, endometrial cancers along with melanomas and glioblastomas (Steck et al, Nat. Genetics, 1997, 15, 356-362).

In vivo evidence from hypomorphic PDK1 knockout mice in a PTEN deficient background, strongly implicate PDK in a wide range of tumour types (Bayascas et al, Curr. Biol., 2005, 15, 1839-1846). Further, in vivo studies with an inhibitor of PDK1, 7-hydroxystauro-sporine, are consistent with these findings (Sato et al, Oncogene, 2002, 21, 1727-1738). Accordingly it is expected that an inhibitor of phosphoinositide dependent protein kinase-1 (PDK1) would be useful in the treatment of diseases such as cancer, for example colon, breast or prostate cancer.

### SUMMARY OF THE INVENTION

In accordance with the present invention, the applicants have hereby discovered novel compounds that are potent inhibitors of the kinase CHK1 and therefore possess the ability to prevent cell cycle arrest at the G2/M checkpoint in response to DNA damage. Certain compounds of the invention are also inhibitors of a PDK1. The compounds of the invention are accordingly useful for their anti-proliferative (such as anti-cancer) activity and are therefore useful in methods of treatment of the human or animal body.

Certain compounds of the invention are also inhibitors of a Pak kinase, for example inhibitors of one or more of Pak 1, Pak 2, Pak 3, Pak 4, Pak 5 and Pak 6 kinase, particularly Pak 1, Pak 2 or Pak 4 Kinase. Compounds with Pak kinase activity are also expected to be useful in the inhibition of tumourigenesis, for example by inhibiting cell survival, cell transformation or cell motility.

The invention also relates to processes for the manufacture of said compounds, to pharmaceutical compositions containing them and to their use in the manufacture of medicaments for use in the production of an anti-cancer effect, for example an anti-proliferative effect, in warm-blooded animals such as man.

The present invention includes pharmaceutically acceptable salts of such compounds. Also in accordance with the present invention applicants provide pharmaceutical compositions and a method to use such compounds in the treatment of cancer.

Such properties are expected to be of value in the treatment of disease states associated with cell cycle arrest, cell proliferation, cell survival, cell transformation or cell motility such as cancers (solid tumors and leukemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, neural degenerative diseases and diseases associated with defective neural regeneration such as Parkinson's disease and Alzheimer's disease, acute and chronic inflammation such as osteoarthritis, rheumatoid arthritis or joint pain, bone diseases and ocular diseases with retinal vessel proliferation.

### DETAILED DESCRIPTION OF THE INVENTION

Accordingly, the present invention provides a compound of formula (I)

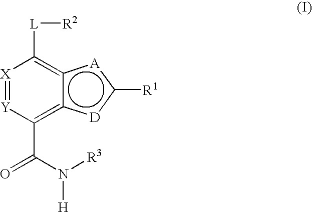

wherein:

A and D are each independently selected from N, CH, S, O and NR⁴;

L is selected from NR⁵, O and S;

X and Y are each independently selected from N and CH;

R¹ is selected from cyano, halo; C₁₋₆alkyl, —NR¹¹R¹², C₁₋₆alkoxy, C₂₋₆alkenyl, C₂₋₆alkynyl, cycloalkyl, cycloalkenyl, aryl, heterocyclyl, OR⁶; —COcarbocyclyl, —COheterocyclyl, —CO(C₁₋₆alkyl), —CONR²⁸R²⁹, —S(O)ₓ(C₁₋₆alkyl), —S(O)ₓcarbocyclyl, —S(O)ₓheterocyclyl, S(O)_{y}NR²⁸R²⁹, and —(C₁₋₆alkyl)S(O)_{y}NR²⁸R²⁹ wherein x is independently 0 to 2 and y is independently 1 or 2; and wherein R¹ may be optionally substituted on one or more carbon atoms by one or more R⁹; and wherein if heterocyclyl contains an —NH— moiety, the nitrogen of said moiety may be optionally substituted by a group selected from R¹⁰;

R² is selected from (C₁₋₃alkyl)NR⁷R⁵, a 4- to 7-membered heterocyclyl ring containing at least one nitrogen atom, —COcarbocyclyl, —COheterocyclyl, —CO(C₁₋₆alkyl), —CONR²⁸R²⁹, —CO₂(C₁₋₆alkyl), —CO₂-carbocyclyl, —CO₂heterocyclyl, —CO₂NR²⁸R²⁹, —S(O)ₓ(C₁₋₆alkyl), S(O)ₓcycloalkyl, —S(O)ₓcycloalkenyl, —S(O)ₓheterocyclyl, S(O)_{y}NR²⁸R²⁹, and —(C₁₋₆alkyl)S(O)_{y}NR²⁸R²⁹ wherein x is independently 0 to 2 and y is independently 1 or 2 and wherein R² may be optionally substituted on one or more carbon atoms by one or more R¹³; and further wherein if heterocyclyl contains an —NH— moiety, the nitrogen of said moiety may be optionally substituted by a group selected from R¹⁴;

R³ is selected from H, benzyl, C₁₋₆alkyl, cycloallkyl, cylcoalkenyl, aryl, heterocyclyl, OR⁶, CHO, —COcarbocyclyl, —CO(C₁₋₆alkyl), —CONR²⁸R²⁹, —S(O)ₓ(C₁₋₆alkyl), —S(O)ₓcarbocyclyl, —S(O)ₓheterocyclyl, S(O)_{y}NR²⁸R²⁹, and —(C₁₋₆alkyl)S(O)_{y}NR²⁸R²⁹ wherein x is independently 0 to 2, y is independently 1 or 2 and wherein R³ may be optionally substituted on one or more carbon atoms by one or more R¹⁵; and wherein if heterocyclyl contains a —NH— moiety, the nitrogen may be optionally substituted by a group selected from R¹⁶;

R⁴ is selected from H, C₁₋₃alkyl, cyclopropyl and CF₃;

R⁵ is selected from H, C₁₋₆alkyl, cycloalkyl, cylcoalkenyl, heterocyclyl and OR⁶; wherein R⁵ may be optionally substituted on carbon by one or more R¹⁷; and wherein if said heterocyclyl contains a —NH— moiety, the nitrogen of said moiety may be optionally substituted by a group selected from R¹⁸;

R⁶ is selected from H, C₁₋₆alkyl, cycloalkyl, cylcoalkenyl, aryl, and heterocyclyl; wherein R⁶ may be optionally substituted on carbon by one or more R¹⁹; and wherein if said heterocyclyl contains a —NH— moiety, the nitrogen of said moiety may be optionally substituted by a group selected from R²⁴;

R⁷ and R⁸ are independently selected from H, C₁₋₆alkyl, cycloalkyl, cylcoalkenyl, aryl, and heterocyclyl; wherein R⁷ and R⁸ independently of each other may be optionally substituted on carbon by one or more R²⁰; and wherein if said heterocyclyl contains a —NH— moiety, the nitrogen of said moiety may be optionally substituted by a group selected from R²¹;

R¹¹ and R¹² are independently selected from H, C₁₋₆alkyl, cycloalkyl, cylcoalkenyl, aryl, heterocyclyl, wherein R¹¹ and R¹² independently of each other may be optionally substituted on carbon by one or more R³²; and wherein if said heterocyclyl contains a —NH— moiety, the nitrogen of said moiety may be optionally substituted by a group selected from R³³;

R⁹, R¹³, R¹⁵, R¹⁷, R¹⁹, R²⁰, R³² and R³⁴ are each independently selected from halo, nitro, —NR²⁸R²⁹, cyano, isocyano, C₁₋₆alkyl, C₂₋₆alkenyl, C₂₋₆alkynyl, aryl, cycloalkyl, cylcoalkenyl, heterocyclyl, hydroxy, keto (═O), —O(C₁₋₆alkyl), —Ocarbocyclyl, —Oheterocyclyl, —Oaryl, —OC(O)C₁₋₆alkyl, —NHCHO, —N(C₁₋₆alkyl)CHO, —NHCONR²⁸R²⁹, —N(C₁₋₆alkyl)CONR²⁸R²⁹, —NHCO(C₁₋₆alkyl), —NHCOcarbocyclyl, —NHCO(heterocyclyl), —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR²⁸R²⁹, —CO(C₁₋₆alkyl), —COheterocyclyl, —COcycloalkyl, —COcycloalkenyl, —COaryl, —CO₂H, —CO₂(C₁₋₆alkyl), —CO₂-carbocyclyl, —CO₂heterocyclyl, —OC(O)(NR²⁸R²⁹), mercapto, —S(O)ₓ(C₁₋₆alkyl), —S(O)ₓcarbocyclyl, —S(O)ₓheterocyclyl, and —S(O)ₓNR²⁸R²⁹; wherein x is independently 0 to 2, wherein R⁹, R¹³, R¹⁵, R¹⁷, R¹⁹, R²⁰, R³² and R³⁴ independently of each other may be optionally substituted on carbon by one or more R²² and wherein if heterocyclyl contains a —NH— moiety, the nitrogen of said moiety may be optionally substituted by a group selected from R²³;

R¹⁰, R¹⁴, R¹⁶, R¹⁸, R²¹, R²⁴, R³³, and R³⁵ are each independently selected from cyano, C₁₋₆alkyl, C₂₋₆alkenyl, C₂₋₆alkynyl, aryl, cycloalkyl, cylcoalkenyl, heterocyclyl, hydroxy, —O(C₁₋₆alkyl), —Ocarbocyclyl, -amidino, —CHO, —CONR²⁸R²⁹, —CO(C₁₋₆alkyl), —COheterocyclyl, —COcarbocyclyl —COaryl, —CO₂(C₁₋₆alkyl), —CO₂-carbocyclyl, —CO₂heterocyclyl, —S(O)ₓ(C₁₋₆alkyl), —S(O)ₓcarbocyclyl, —S(O)ₓheterocyclyl, and —S(O)_{y}NR²⁸R²⁹; wherein x is independently 0 to 2, and y is independently 1 or 2; wherein R¹⁰, R¹⁴, R¹⁶, R¹⁸, R²¹, R²⁴, R³³ and R³⁵ independently of each other may be optionally substituted on carbon by one or more R²⁵ and wherein if said heterocyclyl contains a —NH— moiety, the nitrogen of said moiety may be optionally substituted by a group selected from R²⁶;

R²² and R²⁵ are each independently selected from halo, nitro, —NR²⁸R²⁹, cyano, isocyano, C₁₋₆alkyl, C₂₋₆alkenyl, C₂₋₆alkynyl, aryl, cycloalkyl, cylcoalkenyl, heterocyclyl, hydroxy, keto(═O), —O(C₁₋₆alkyl), —Ocarbocyclyl, —Oheterocyclyl, —Oaryl, —OC(O)C₁₋₆alkyl, —NHCHO, —N(C₁₋₆alkyl)CHO, —NHCONR²⁸R²⁹, —N(C₁₋₆alkyl)CONR²⁸R²⁹, —NHCO(C₁₋₆alkyl), —NHCOcarbocyclyl, —NHCO(heterocyclyl), —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR²⁸R²⁹, —CO(C₁₋₆alkyl), —COheterocyclyl, —COcycloalkyl, —COcycloalkenyl, —CO₂H, —CO₂(C₁₋₆alkyl), —CO₂-carbocyclyl, —OC(O)(NR²⁸R²⁹), mercapto, —S(O)ₓ(C₁₋₆alkyl), —S(O)ₓcarbocyclyl, —S(O)ₓheterocyclyl, and —S(O)ₓNR²⁸R²⁹; wherein x is independently 0 to 2, wherein R²² and R²⁵ may be optionally substituted on carbon by one or more R³⁶ and wherein if said heterocyclyl contains a —NH— moiety, the nitrogen of said moiety may be optionally substituted by a group selected from R²⁷;

R²³ and R²⁶ are each independently selected from cyano, C₁₋₆alkyl, C₂₋₆alkenyl, C₂₋₆alkynyl, aryl, cycloalkyl, cylcoalkenyl, heterocyclyl, hydroxy, —O(C₁₋₆alkyl), —Ocarbocyclyl, -amidino, —CHO, —CONR²⁸R²⁹, —CO(C₁₋₆alkyl), —COheterocyclyl, —COcycloalkyl, —COcycloalkenyl, —CO₂(C₁₋₆allyl), —CO₂-carbocyclyl, —S(O)ₓ(C₁₋₆alkyl), —S(O)ₓcarbocyclyl, —S(O)ₓheterocyclyl, and —S(O)_{y}NR²⁸R²⁹; wherein x is independently 0 to 2, and y is independently 1 or 2; wherein R²³ and R²⁶ independently of each other may be optionally substituted on carbon by one or more R³⁰ and wherein if said heterocyclyl contains a —NH—moiety, the nitrogen of said moiety may be optionally substituted by a group selected from R³¹;

R²⁸ and R²⁹ are each independently selected from H, amino, cyano, C₁₋₆alkyl, C₂₋₆alkenyl, C₂₋₆alkynyl, aryl, cycloalkyl, cycloalkenyl, heterocyclyl, hydroxy, —O(C₁₋₆alkyl), —Oaryl, —OCOalkyl, -amidino, —CHO, —CO(C₁₋₆alkyl), —COheterocyclyl, —COcycloalkyl, —COcycloalkenyl, —SO(C₁₋₆alkyl), —SO₂(C₁₋₆alkyl), wherein R²⁸ and R²⁹ independently of each other may be optionally substituted on carbon by one or more R³⁴; and wherein if said heterocyclyl contains a —NH— the nitrogen of said moiety may be optionally substituted by a group selected from R³⁵;

R³⁰ and R³⁶ are each independently selected from halo, nitro, —NR²⁸R²⁹, cyano, isocyano, C₁₋₆alkyl, C₂₋₆alkenyl, C₂₋₆alkynyl, aryl, cycloalkyl, cylcoalkenyl, heterocyclyl, hydroxy, keto (═O), —O(C₁₋₆alkyl), —Ocarbocyclyl, —OC(O)C₁₋₆alkyl, —NHCHO, —N(C₁₋₆alkyl)CHO, —NHCONR²⁸R²⁹, —N(C₁₋₆alkyl)CONR²⁸R²⁹, —NHCO(C₁₋₆alkyl), —NHCOcarbocyclyl, —NHCO(heterocyclyl), —NHCO₂(C₁₋₆alkyl); —NHCO₂H, —N(C₁₋₆alkyl)CO(C₁₋₆allyl), —NHSO₂(C₁₋₆alkyl), carboxy, -amidino, —CHO, —CONR²⁸R²⁹, —CO(C₁₋₆alkyl), —COheterocyclyl, —COcycloallyl, —COcycloalkenyl, —CO₂H, —CO₂(C₁₋₆alkyl), —CO₂-carbocyclyl, —OC(O)(NR²⁸R²⁹), mercapto, —S(O)ₓ(C₁₋₆alkyl), —S(O)ₓcarbocyclyl, —S(O)ₓheterocyclyl, and —S(O)ₓNR²⁸R²⁹; wherein x is independently 0 to 2;

R²⁷ and R³¹ are each independently selected from cyano, C₁₋₆alkyl, C₂₋₆alkenyl, C₂₋₆alkynyl, aryl, cycloalkyl, cylcoalkenyl, heterocyclyl, hydroxy, —O(C₁₋₆alkyl), —Ocarbocyclyl, —(C₁₋₆alkyl)-O—(C₁₋₆alkyl), -amidino, —CHO, —CONR²⁸R²⁹, —CO(C₁₋₆alkyl), —COheterocyclyl, —COcycloalkyl, —COcycloalkenyl, —CO₂(C₁₋₆alkyl), —CO₂-carbocyclyl, —S(O)ₓ(C₁₋₆alkyl), —S(O)ₓcarbocyclyl, —S(O)ₓheterocyclyl, and —S(O)_{y}NR²⁸R²⁹; wherein x is independently 0 to 2, and y is independently 1 or 2;

or a pharmaceutically acceptable salt thereof.

As used in this application, the term “optionally substituted,” means that substitution is optional and therefore it is possible for the designated atom to be unsubstituted. In the event a substitution is desired then such substitution means that any number of hydrogens on the designated atom is replaced with a selection from the indicated group, provided that the normal valency of the designated atom is not exceeded, and that the substitution results in a stable compound.

When a circle is shown within a ring structure, it indicates that the ring system is aromatic.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure resulting from the removal of one or more hydrogens from a hydrocarbon.

The term “alkyl” used alone or as a suffix or prefix, refers to monovalent straight or branched chain hydrocarbon radicals comprising 1 to about 12 carbon atoms unless otherwise specified and includes both straight and branched chain alkyl groups. References to individual alkyl groups such as “propyl” are specific for the straight chain version only and references to individual branched chain alkyl groups such as ‘isopropyl’ are specific for the branched chain version only. For example, “C₁₋₆alkyl” includes C₁₋₄alkyl, C₁₋₃alkyl, propyl, isopropyl and t-butyl. A similar convention applies to other radicals, for example “phenylC₁₋₆alkyl” includes phenylC₁₋₄alkyl, benzyl, 1-phenylethyl and 2-phenylethyl.

The term “alkenyl” used alone or as suffix or prefix, refers to a monovalent straight or branched chain hydrocarbon radical having at least one carbon-carbon double bond and comprising at least 2 up to about 12 carbon atoms unless otherwise specified.

The term “alkynyl” used alone or as suffix or prefix, refers to a monovalent straight or branched chain hydrocarbon radical having at least one carbon-carbon triple bond and comprising at least 2 up to about 12 carbon atoms unless otherwise specified.

The term “cycloalkyl,” used alone or as suffix or prefix, refers to a saturated, monovalent ring-containing hydrocarbon radical comprising at least 3 up to about 12 carbon atoms. When cycloalkyl contains more than one ring, the rings may be fused or unfused and include bicyclo radicals. Fused rings generally refer to at least two rings sharing two atoms therebetween. Exemplary cycloalkyl include cyclopropyl, cyclobutyl, cyclopentyl, cyclohexyl and norboranyl.

The term “cycloalkenyl” used alone or as suffix or prefix, refers to a monovalent ring-containing hydrocarbon radical having at least one carbon-carbon double bond and comprising at least 3 up to about 12 carbon atoms but excluding aromatic ring systems. When cycloalkenyl contains more than one ring, the rings may be fused or unfused and include bicyclo radicals. Exemplary cycloalkenyl includes cyclohexenyl and cycloheptenyl.

The term “aryl” used alone or as suffix or prefix, refers to a hydrocarbon radical having one or more polyunsaturated carbon rings having aromatic character, (e.g., 4n+2 delocalized electrons) and comprising 6 up to about 14 carbon atoms, wherein the radical is located on a carbon of the aromatic ring. Exemplary aryl includes phenyl, naphthyl, and indenyl.

The term “alkoxy” used alone or as a suffix or prefix, refers to radicals of the general formula —O—R, wherein -R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “carbocyclyl” refers to saturated, partially saturated and unsaturated, mono, bi or polycyclic carbon rings. These may include fused or bridged bi- or polycyclic systems. Carbocyclyls may have from 3 to 12 carbon atoms in their ring structure, i.e. C₃₋₁₂-carbocyclyl, and in a particular embodiment are monocyclic rings have 3 to 7 carbon atoms or bicyclic rings having 7 to 10 carbon atoms in the ring structure. Examples suitable carbocyclyls include cyclopropyl, cyclobutyl, cyclopentyl, cyclohexyl, cycloheptyl, cyclohexenyl, cyclopentadienyl, indanyl, phenyl and naphthyl.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)— and a ring sulphur atom may be optionally oxidised to form the S-oxides. Heterocyclyl may contain more than one ring. When a heterocyclyl contains more than one ring, the rings may be fused. Fused rings generally refer to at least two rings sharing two atoms there between. Heterocyclyl may be aromatic. Examples of heterocyclyls include, but are not limited to, 1H-indazolyl, 2-pyrrolidonyl, 2H, 6H-1,5,2-dithiazinyl, 2H-pyrrolyl, 3H-indolyl, 4-piperidonyl, 4aH-carbazolyl, 4H-quinolizinyl, 6H-1,2,5-thiadiazinyl, acridinyl, azepanyl, azetidinyl, aziridinyl, azocinyl, benzimidazolyl, benzofuranyl, benzofuranyl, benzothiofuranyl, benzothiophenyl, benzodioxolyl, benzoxazinyl, dihydrobenzoxazinyl, 3,4-dihydro-1,4-benzoxazinyl, benzoxazolyl, benzthiophenyl, benzthiazolyl, benzotriazolyl, benzotetrazolyl, benzisoxazolyl, benzthiazole, benzisothiazolyl, benzimidazolyls, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dioxolanyl, furyl, 2,3-dihydrofuranyl, 2,5-dihydrofuranyl, dihydrofuro[2,3-b]tetrahydrofuranyl, furanyl, furazanyl, homopiperidinyl, imidazolyl, imidazolidinyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxiranyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, piperidinyl, pteridinyl, piperidonyl, 4-piperidonyl, purinyl, pyranyl, pyrrolidinyl, pyrrolinyl, pyrrolidinyl, pyrazinyl, pyrazolidinyl, pyrazolinyl, pyrazolyl, pyridazinyl, pyridooxazolyl, pyridoimidazolyl, pyridothiazolyl, pyridinyl, N-oxide-pyridinyl, pyridyl, pyrimidinyl, pyrrolidinyl, pyrrolinyl, pyrrolyl, pyridinyl, quinazolinyl, quinolinyl, 4H-quinolizinyl, quinoxalinyl, quinuclidinyl, carbolinyl, tetrahydrofuranyl, tetrahydroisoquinolinyl, thiophanyl, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, thiiranyl, triazinyl, 1,2,3-triazolyl, 1,2,4-triazolyl, 1,2,5-triazolyl, 1,3,4-triazolyl, and xanthenyl. In one aspect of the invention a “heterocyclyl” is a saturated, partially saturated or unsaturated, monocyclic ring containing 5, 6 or 7 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides. Particular examples of heterocyclyl include azepanyl, 1H-indazolyl, piperidinyl, 1H-pyrazolyl, pyrimidyl, pyrrolidinyl, pyridinyl and thienyl.

As used herein, “4- to 7-membered heterocyclyl ring containing at least one nitrogen atom” means a 4-, 5-, 6- or 7-membered heterocycly ring containing at least one nitrogen atom. Exemplary 4- to 7-membered heterocycly rings containing at least one nitrogen include, but are not limited to, piperidinyl, azetidinyl, azepanyl, pyrrolidinyl, pyrazolidinyl, piperazinyl, imidazolyl, morpholinyl, indolinyl, and thiomorpholinyl.

The term “halo” means fluoro, chloro, bromo and iodo.

When any variable (e.g., R²⁸, R²⁹ etc.) occurs more than one time in any formula for a compound, its definition at each occurrence is independent of its definition at every other occurrence.

Some of the compounds of formula (I) may have chiral centers and/or geometric isomeric centers (E- and Z-isomers) and therefore the compounds may exist in particular stereoisomeric or geometric forms. It is to be understood that the present invention encompasses all such optical, diastereoisomers and geometric isomers and mixtures thereof that possess CHK1, Pak or PDK1 kinase inhibitory activity. The present invention also encompasses all tautomeric forms of the compounds of formula (I) that possess CHK 1, Pak or PDK1 kinase inhibitory activity. It is well known in the art how to prepare optically active forms, such as by resolution of racemic forms or by synthesis from optically active starting materials. When required, separation of the racemic material can be achieved by methods known in the art. All chiral, diastereomeric, racemic forms and all geometric isomeric forms of a structure are intended, unless the specific stereochemistry or isomeric form is specifically indicated.

The following substituents for the variable groups contained in formula (I) are further embodiments of the invention. Such specific substituents may be used, where appropriate, with any of the definitions, claims or embodiments defined hereinbefore or hereinafter.

X is N.

Y is CH.

X is CH and Y is CH.

D is S.

A is S.

A is N.

A is NR⁵

D is N.

D is NR⁵

A is O.

D is O.

A is N and D is O.

A is S and D is N.

X is N and A is S.

X is N and D is S.

X is N and A is O.

X is N and D is O.

X is N; A is S; and Y is CH.

X is N; D is S; and Y is CH.

X is N; A is S; D is CH and Y is CH.

X is N; D is S; A is CH and Y is CH.

At least one of A or D is S.

X is N; A is S; D is N and Y is CH.

X is N; D is S; A is N and Y is CH.

A is CH; D is NR⁴; X is CH; and Y is CH.

A is CH; D is NH; X is CH; and Y is CH.

L is NR⁵.

L is NR⁵ and R⁵ is H.

L is NR⁵ and R⁵ is cyclopropyl wherein R⁵ may be optionally substituted on carbon by one or more R¹⁷.

L is NR⁵ and R⁵ is H or C₁₋₃ alkyl wherein R⁵ may be optionally substituted on carbon by one or more R¹⁷.

L is NH.

L is O.

L is S.

R¹ is selected from C₁₋₆alkyl, C₂₋₆alkenyl, C₂₋₆alkynyl, aryl, and heterocyclyl wherein R¹ may be optionally substituted on one or more carbon atoms by one or more R⁹; and wherein if heterocyclyl contains an —NH— moiety, the nitrogen of said moiety may be optionally substituted by a group selected from R¹⁰.

R¹ is aryl wherein R¹ may be optionally substituted on one or more carbon atoms by one or more R⁹.

R¹ is aryl wherein R¹ may be optionally substituted on one or more carbon atoms by one or more R⁹ wherein R⁹ is selected from the group consisting of halo, C₁₋₆alkyl, C₂₋₆alkenyl, C₂₋₆alkynyl, heterocyclyl, —O(C₁₋₆alkyl), —CO(C₁₋₆alkyl), —CONR²⁸R²⁹, and —NHCO(heterocyclyl) wherein R⁹ may be optionally substituted on carbon by one or more R²² and wherein if heterocyclyl contains a —NH— moiety, the nitrogen of said moiety may be optionally substituted by a group selected from R²³.

R¹ is heterocyclyl wherein R¹ may be optionally substituted on one or more carbon atoms by one or more R⁹; and wherein if heterocyclyl contains an —NH— moiety, the nitrogen of said moiety may be optionally substituted by a group selected from R¹⁰.

R¹ is aryl wherein R¹ may be optionally substituted on one or more carbon atoms by one or more R⁹ wherein R⁹ is selected from the group consisting of halo and C₁₋₆alkyl, and wherein R⁹ may be optionally substituted on carbon by one or more R²² wherein R²² is selected from halo, —NR²⁸R²⁹, cyano, isocyano, aryl, cycloalkyl, cylcoalkenyl, and; wherein R²² may be optionally substituted on carbon by one or more R³⁶ and wherein if said heterocyclyl contains a —NH— moiety, the nitrogen of said moiety may be optionally substituted by a group selected from R²⁷.

R¹ is aromatic heterocyclyl wherein R¹ may be optionally substituted on one or more carbon atoms by one or more R⁹; and wherein if heterocyclyl contains an —NH— moiety, the nitrogen of said moiety may be optionally substituted by a group selected from R¹⁰.

R¹ is selected from benzimidazolyl, benzoxazinyl, dihydrobenzoxazinyl, imidazolinyl, thienyl, pyrazolyl; pyradinyl and pyrimidinyl wherein R¹ may be optionally substituted on one or more carbon atoms by one or more R⁹; and wherein if heterocyclyl contains an —NH— moiety, the nitrogen of said moiety may be optionally substituted by a group selected from R¹⁰.

R² is a 4- to 7-membered heterocyclyl ring containing at least one nitrogen atom wherein said heterocyclyl may be optionally substituted on one or more carbon atoms by one or more R¹³; and further wherein if said heterocyclyl contains an —NH— moiety, the nitrogen of said moiety may be optionally substituted by a group selected from R¹⁴.

R² is a 4- to 7-membered saturated heterocyclyl ring containing at least one nitrogen atom wherein said heterocyclyl may be optionally substituted on one or more carbon atoms by one or more R¹³; and further wherein if said heterocyclyl contains an —NH— moiety, the nitrogen of said moiety may be optionally substituted by a group selected from R¹⁴.

R² is a 4-membered heterocyclyl ring containing at least one nitrogen atom wherein said heterocyclyl may be optionally substituted on one or more carbon atoms by one or more R¹³; and further wherein if said heterocyclyl contains an —NH— moiety, the nitrogen of said moiety may be optionally substituted by a group selected from R¹⁴.

R² is a 5-membered heterocyclyl ring containing at least one nitrogen atom wherein said heterocyclyl may be optionally substituted on one or more carbon atoms by one or more R¹³; and further wherein if said heterocyclyl contains an —NH— moiety, the nitrogen of said moiety may be optionally substituted by a group selected from R¹⁴.

R² is a 6-membered heterocyclyl ring containing at least one nitrogen atom wherein said heterocyclyl may be optionally substituted on one or more carbon atoms by one or more R¹³; and further wherein if said heterocyclyl contains an —NH— moiety, the nitrogen of said moiety may be optionally substituted by a group selected from R¹⁴.

R² is a 7-membered heterocyclyl ring containing at least one nitrogen atom wherein said heterocyclyl may be optionally substituted on one or more carbon atoms by one or more R¹³; and further wherein if said heterocyclyl contains an —NH— moiety, the nitrogen of said moiety may be optionally substituted by a group selected from R¹⁴.

R² is selected from the group consisting of piperidinyl, azetidinyl, azepanyl, pyrrolidinyl, pyrazolidinyl, piperazinyl, imidazolyl, morpholinyl, indolinyl, and thiomorpholinyl wherein said piperidinyl, azetidinyl, azepanyl, pyrrolidinyl, pyrazolidinyl, piperazinyl, imidazolyl, morpholinyl, indolinyl, and thiomorpholinyl may be optionally substituted on one or more carbon atoms by one or more R¹³; and further wherein said piperidinyl, azepanyl, pyrrolidinyl, pyrazolidinyl, piperazinyl, imidazolyl, morpholinyl, indolinyl, and thiomorpholinyl may be optionally substituted on N by a group selected from R¹⁴.

R² is selected from the group consisting of pyrrolidin-3-yl, piperidin-3-yl, and azepan-3-yl wherein said pyrrolidin-3-yl, piperidin-3-yl, and azepan-3-yl may be optionally substituted on one or more carbon atoms by one or more R¹³; and further wherein said pyrrolidin-3-yl, piperidin-3-yl, or azepan-3-yl may be optionally substituted on N by a group selected from R¹⁴.

R³ is selected from H, benzyl, C₁₋₆alkyl, cycloalkyl, cylcoalkenyl, aryl, heterocyclyl and OR⁶, wherein R³ may be optionally substituted on one or more carbon atoms by one or more R¹⁵; and wherein if heterocyclyl contains a —NH— moiety, the nitrogen may be optionally substituted by a group selected from R¹⁶.

R³ is pyrazinyl optionally substituted on one or more carbon atoms by one or more R¹⁵.

R³ is H.

R³ is methyl.

R⁴ is H.

In a further aspect of the present invention there is provided a compound of formula (I) wherein:

A is CH;

D is S;

L is NR⁵;

X is N;

Y is CH;

R¹ is selected from C₁₋₆alkyl, aryl and heterocyclyl wherein R¹ may be optionally substituted on one or more carbon atoms by one or more R⁹; and wherein if heterocyclyl contains an —NH— moiety, the nitrogen of said moiety may be optionally substituted by a group selected from R¹⁰;

R² is a 4- to 7-membered heterocyclyl ring containing at least one nitrogen atom, wherein R² may be optionally substituted on one or more carbon atoms by one or more R¹³; and further wherein if heterocyclyl contains an —NH— moiety, the nitrogen of said moiety may be optionally substituted by a group selected from R¹⁴;

R³ is H;

R⁵ is H or C₁₋₃alkyl; or a pharmaceutically acceptable salt thereof.

In a further aspect of the present invention there is provided a compound of formula (I) wherein:

A is CH;

D is S;

L is NR⁵;

X is N;

Y is CH;

R¹ is selected from aryl and heterocyclyl wherein R¹ may be optionally substituted on one or more carbon atoms by one or more R⁹; and wherein if heterocyclyl contains an —NH— moiety, the nitrogen of said moiety may be optionally substituted by a group selected from R¹⁰;

R² is (C₁₋₃alkyl)NR⁷R⁸, wherein R² may be optionally substituted on one or more carbon atoms by one or more R¹³;

R³ is H;

R⁵ is H or C₁₋₃alkyl;

R⁷ and R⁸ are independently selected from H, C₁₋₆alkyl, cycloalkyl, cycloalkenyl, aryl, and heterocyclyl; wherein R⁷ and R⁸ independently of each other may be optionally substituted on carbon by one or more R²⁰; and wherein if said heterocyclyl contains a —NH-moiety, the nitrogen of said moiety may be optionally substituted by a group selected from R²¹;

or a pharmaceutically acceptable salt thereof.

In a still further aspect of the present invention there is provided a compound of formula (I) wherein:

A is CH;

D is S;

L is NR⁵;

X is N;

Y is CH;

R¹ is selected from aryl and heterocyclyl wherein R¹ may be optionally substituted on one or more carbon atoms by one or more R⁹; and wherein if heterocyclyl contains an —NH— moiety, the nitrogen of said moiety may be optionally substituted by a group selected from R¹⁰;

R² is a 4- to 7-membered heterocyclyl ring containing at least one nitrogen atom, wherein R² may be optionally substituted on one or more carbon atoms by one or more R¹³; and further wherein if heterocyclyl contains an —NH— moiety, the nitrogen of said moiety may be optionally substituted by a group selected from R¹⁴;

R³ is H;

R⁵ is H or C₁₋₃alkyl; or a pharmaceutically acceptable salt thereof.

In a further aspect of the present invention there is provided a compound of formula (I) wherein:

A is CH;

D is NR¹;

L is NR⁵;

X is CH;

Y is CH;

R¹ is selected from C₁₋₆alkyl, aryl and heterocyclyl wherein R¹ may be optionally substituted on one or more carbon atoms by one or more R⁹; and wherein if heterocyclyl contains an —NH— moiety, the nitrogen of said moiety may be optionally substituted by a group selected from R¹⁰;

R² is a 4- to 7-membered heterocyclyl ring containing at least one nitrogen atom, wherein R² may be optionally substituted on one or more carbon atoms by one or more R¹³; and further wherein if heterocyclyl contains an —NH— moiety, the nitrogen of said moiety may be optionally substituted by a group selected from R¹⁴;

R³ is H;

R⁴ is H, C₁₋₃alkyl, cyclopropyl and CF₃;

R⁵ is H or C₁₋₃alkyl; or a pharmaceutically acceptable salt thereof.

In a further aspect of the present invention there is provided a compound of formula (I) wherein:

A is CH;

D is NR⁴;

L is NR⁵;

X is CH;

Y is CH;

R¹ is selected from aryl and heterocyclyl wherein R¹ may be optionally substituted on one or more carbon atoms by one or more R⁹; and wherein if heterocyclyl contains an —NH— moiety, the nitrogen of said moiety may be optionally substituted by a group selected from R¹⁰;

R² is (C₁₋₃alkyl)NR⁷R⁸, wherein R² may be optionally substituted on one or more carbon atoms by one or more R¹³;

R³ is H;

R⁴ is H, C₁₋₃alkyl, cyclopropyl and CF₃;

R⁵ is H or C₁₋₃alkyl;

R⁷ and R⁸ are independently selected from H, C₁₋₆alkyl, cycloalkyl, cycloalkenyl, aryl, and heterocyclyl; wherein R⁷ and R⁸ independently of each other may be optionally substituted on carbon by one or more R²⁰; and wherein if said heterocyclyl contains a —NH— moiety, the nitrogen of said moiety may be optionally substituted by a group selected from R¹;

or a pharmaceutically acceptable salt thereof.

In a still further aspect of the present invention there is provided a compound of formula (I) wherein:

A is CH;

D is NR⁴;

L is NR⁵;

X is CH;

Y is CH;

R¹ is selected from aryl and heterocyclyl wherein R¹ may be optionally substituted on one or more carbon atoms by one or more R⁹; and wherein if heterocyclyl contains an —NH— moiety, the nitrogen of said moiety may be optionally substituted by a group selected from R¹⁰;

R² is a 4- to 7-membered heterocyclyl ring containing at least one nitrogen atom, wherein R² may be optionally substituted on one or more carbon atoms by one or more R¹³; and further wherein if heterocyclyl contains an —NH— moiety, the nitrogen of said moiety may be optionally substituted by a group selected from R¹⁴;

R³ is H;

R⁴ is H, C₁₋₃alkyl, cyclopropyl and CF₃;

R⁵ is H or C₁₋₃alkyl; or a pharmaceutically acceptable salt thereof.

In a further embodiment of the invention, particularly useful compounds of the invention are any one of the Examples or a pharmaceutically acceptable salt thereof.

An additional embodiment of the present invention is directed to a process for the preparation of a compound of formula (I) wherein X is N, Y is CH, A is CH, D is S, R³ is H and L is NR⁵, or a pharmaceutically acceptable salt thereof, which comprises:
a. reacting a compound of formula (II) wherein Z is halo, e.g. bromo, chloro or iodo

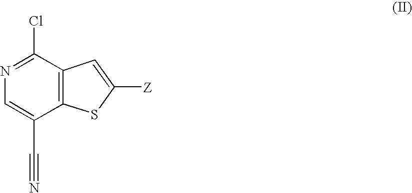

with an amine of formula (III), wherein R² and R⁵ are as defined in formula (I), in the presence of a base

NHR²R⁵ (III)
to yield a compound of formula (IV)

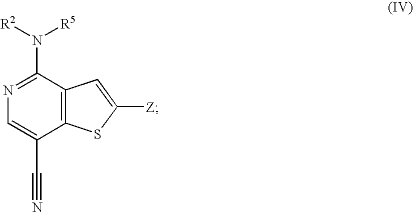

b. reacting the compound of formula (IV) with a compound of formula (V) or (V′), wherein R¹ is defined in formula (I) and R¹ is H or methyl,

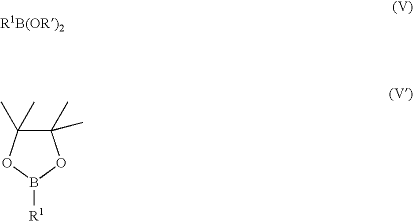

to yield a compound of formula (VI)

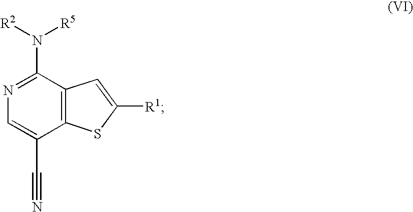

c. hydrolyzing the compound of formula (VI) to form a compound according to formula (I) as shown is formula (IA)

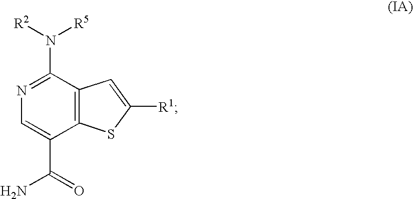

d. and thereafter if necessary:
   i) converting a compound of the formula (I) into another compound of the formula (I);
   ii) removing any protecting groups;
   iii) forming a pharmaceutically acceptable salt.

Another additional embodiment of the present invention is directed to a process for the preparation of a compound of formula (I) wherein X is N, Y is CH, A is CH, D is S, R³ is H, and L is O, or a pharmaceutically acceptable salt thereof, which comprises:
a. reacting a compound of formula (II) wherein Z is halo, e.g. bromo, chloro or iodo

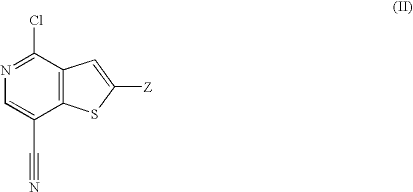

with an alcohol of formula (III′), wherein R² is as defined in formula (I), in the presence of a base, e.g. sodium hydride,

R²OH (III′)
to yield a compound of formula (IV′)

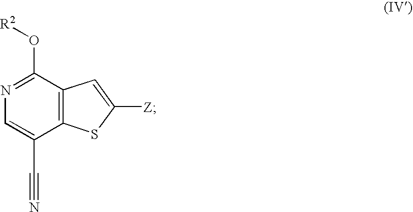

b. reacting the compound of formula (IV′) with a compound of formula (V) or (V′), wherein R¹ is defined in formula (I) and R′ is H or methyl,

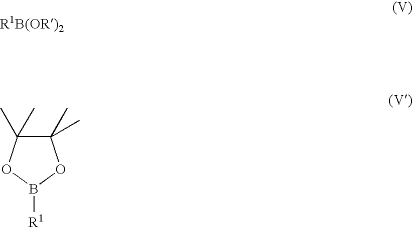

to yield a compound of formula (VI′)

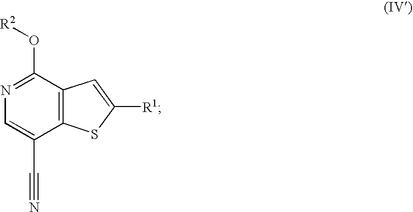

c. hydrolyzing the compound of formula (VI′) to form a compound according to formula (I) as shown in formula (IB)

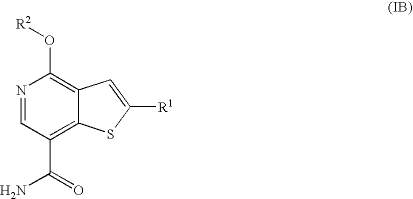

d. and thereafter if necessary:
i) converting a compound of the formula (I) into another compound of the formula (I);
ii) removing any protecting groups;
iii) forming a pharmaceutically acceptable salt.

A still further embodiment of the present invention is directed to a process for the preparation of a compound of formula (I) wherein X is CH, Y is CH, A is CH, D is NR⁴, and L is NR⁵ and R⁵ is H, or a pharmaceutically acceptable salt thereof, which comprises:
a. reacting a compound of formula (VII) wherein R″ is H, methyl, ethyl, or benzyl

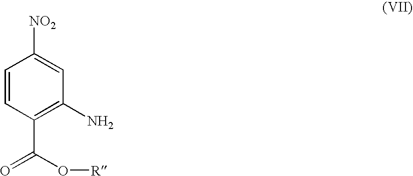

with a ketone of formula (VIII), wherein R¹ is defined in formula (I)

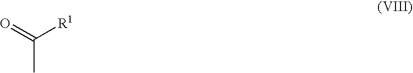

to yield an indole of formula (IX)

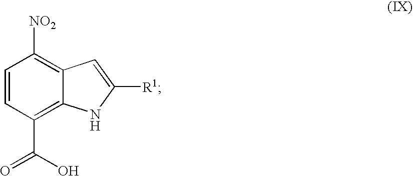

b. reacting the indole of formula (IX) with an amine of formula (X), wherein R³ is defined in formula (I)

R³NH₂ (X)
to yield a compound of formula (XI)

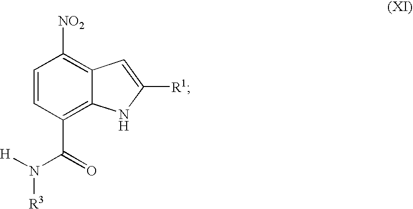

c. reducing the compound of formula (XI) to form the amine of formula (XII)

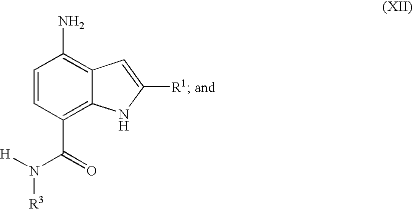

d. reacting the compound of formula (XII) with the appropriate aldehyde, ketone, carboxylic acid or sulfonyl chloride of R², wherein R¹ is defined in formula (I) to form a compound according to formula (I) as shown is formula (IC)

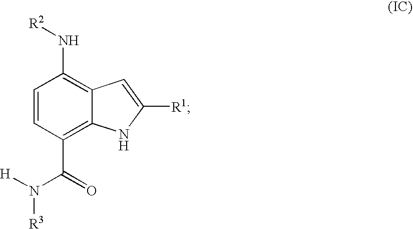

or alternatively, reacting the compound of formula (XII) with sodium nitrite and a copper halide to form a compound of formula (XIII), wherein Z is halo, e.g. bromo, chloro or iodo,

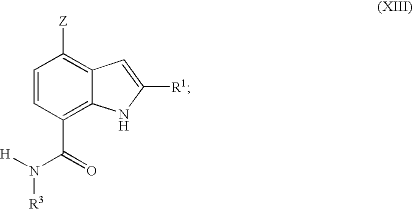

e. reacting a compound of formula (XIII) with an amine of formula (III), wherein R² and R⁵ are as defined in formula (I), in the presence of a catalyst, e.g. palladium or copper derived,

NHR²R⁵ (III)
to yield a compound according to formula (I) as shown in formula (ID)

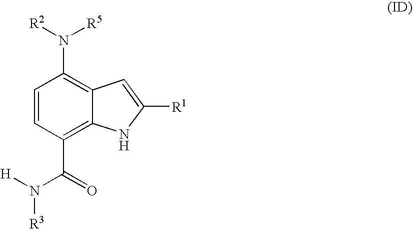

d. and thereafter if necessary:
   i) converting a compound of the formula (I) into another compound of the formula (I);
   ii) removing any protecting groups;
   iii) forming a pharmaceutically acceptable salt of the compounds of formula (IC) or (ID).

It will also be appreciated that in some of the reactions mentioned hereinbefore and after it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green and P. G. M. Wuts, Protective Groups in Organic Synthesis, 3^{rd} Edition, John Wiley and Sons, 1999). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group that may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

In an additional embodiment, the present invention is directed to compounds of the foregoing formula (IV), (IV′), (VI), (VI′), (IX), (XI), (XII), and (XIII) useful as intermediates in the production of compounds according to formula (I).

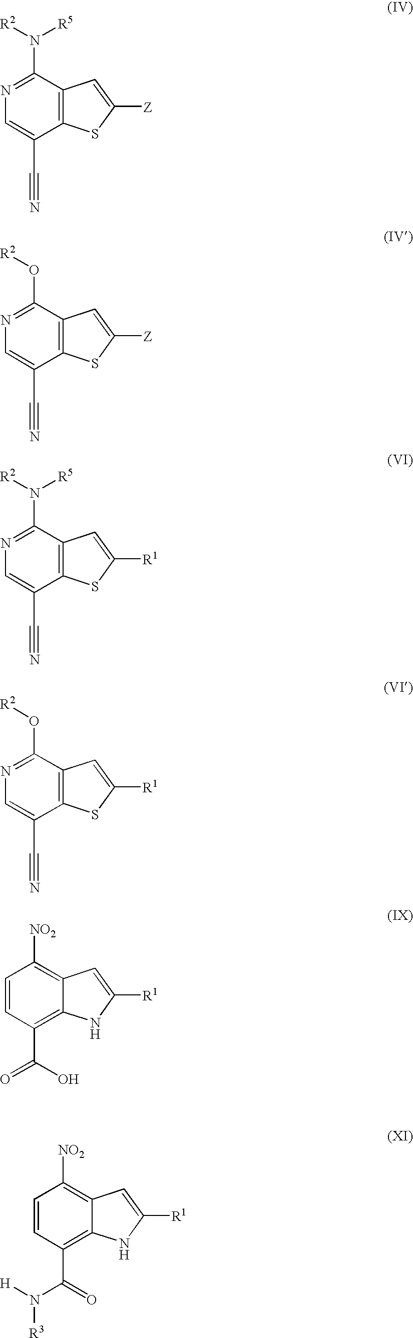

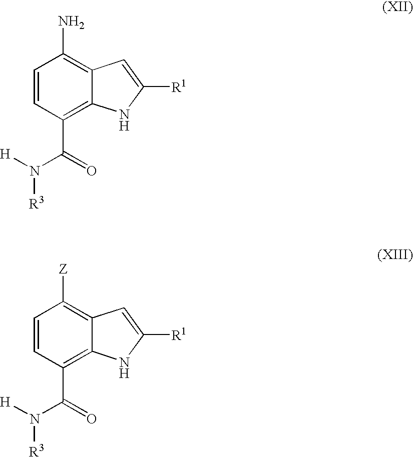

wherein R¹, R², R³, and R⁵ are as defined in formula (I), and Z is halo, e.g. bromo, chloro, and iodo.

In a further embodiment, the present invention is directed to compounds of formula (I) as shown in formula (IA), (IB), (IC), and (ID)

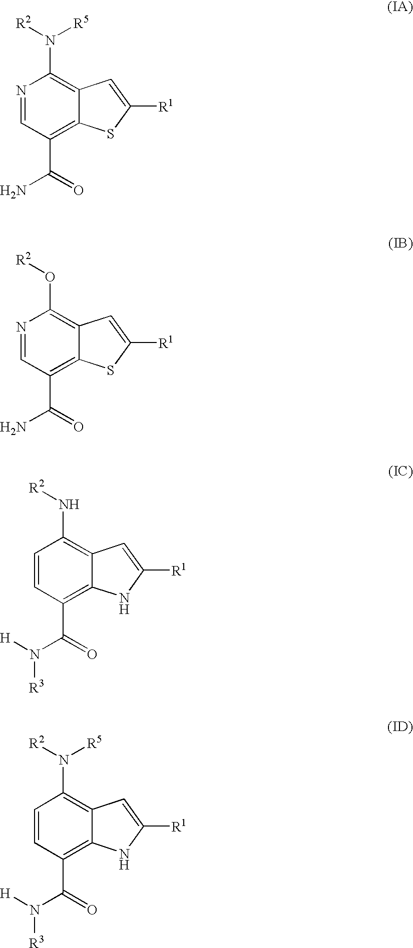

wherein the variable groups are as defined in formula (I) and pharmaceutically acceptable salts thereof.

According to a further aspect of the present invention there is provided a pharmaceutical composition comprising a compound of formula (I) or a pharmaceutically acceptable salt thereof together with at least one pharmaceutically acceptable carrier, diluent or excipient.

In another aspect of the present invention there is provided a compound of formula (I), or a pharmaceutically acceptable salt thereof, as defined hereinbefore for use as a medicament.

In another embodiment the present invention provides the use of a compound of formula (I) or a pharmaceutically acceptable salt thereof in the preparation of a medicament.

In another embodiment the present invention provides the use of a compound of formula (I) or a pharmaceutically acceptable salt thereof in the preparation of a medicament for the treatment or prophylaxis of cancer.

In another embodiment the present invention provides the use of a compound of formula (I) or a pharmaceutically acceptable salt thereof in the preparation of a medicament for the treatment or prophylaxis of neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system, and other tumor types such as melanoma, multiple myeloma, fibrosarcoma and osteosarcoma, and malignant brain tumors.

In still another embodiment the present invention provides the use of a compound of formula (I) or a pharmaceutically acceptable salt thereof in the preparation of a medicament for the treatment or prophylaxis of proliferative diseases including autoimmune, inflammatory, neurological, and cardiovascular diseases.

In another embodiment the present invention provides the use of a compound of formula (I) or a pharmaceutically acceptable salt thereof in the preparation of a medicament for use in the inhibition of CHK1 kinase activity.

In another embodiment the present invention provides the use of a compound of formula (I) or a pharmaceutically acceptable salt thereof in the preparation of a medicament for use in the inhibition of Pak kinase activity, for example inhibition of Pak1, Pak2 or Pak4 kinase activity.

In another embodiment the present invention provides the use of a compound of formula (I) or a pharmaceutically acceptable salt thereof in the preparation of a medicament for use in the inhibition of PDK1 kinase activity.

In another embodiment the present invention provides the use of a compound of formula (I) or a pharmaceutically acceptable salt thereof in the preparation of a medicament for use in limiting cell proliferation.

In another embodiment the present invention provides the use of a compound of formula (I) or a pharmaceutically acceptable salt thereof in the preparation of a medicament for use in limiting tumourigenesis.

In another aspect of the present invention there is provided a compound of formula (I), or a pharmaceutically acceptable salt thereof, as defined hereinbefore for use in a method of treatment of the human or animal body by therapy.

In another embodiment the present invention provides a compound of formula (I) or a pharmaceutically acceptable salt thereof for use in the treatment or prophylaxis of disorders associated with cancer.

In another embodiment the present invention provides a compound of formula (I) or a pharmaceutically acceptable salt thereof for the use in treatment or prophylaxis of neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system, and other tumor types such as melanoma, multiple myeloma, fibrosarcoma and osteosarcoma, and malignant brain tumors.

In another embodiment the present invention provides a compound of formula (I) or a pharmaceutically acceptable salt thereof for use in the treatment or prophylaxis of proliferative diseases including autoimmune, inflammatory, neurological, and cardiovascular diseases.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of a CHK1 kinase inhibitory effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of a Pak kinase inhibitory effect (for example a Pak1, Pak2 or Pak4 kinase inhibitory effect) in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of a PDK1 kinase inhibitory effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment or prophylaxis of proliferative diseases including autoimmune, inflammatory, neurological, and cardiovascular diseases in a warm-blooded animal such as man.

In another embodiment the present invention provides a method of limiting cell proliferation in a human or animal comprising administering to said human or animal a therapeutically effective amount of a compound of formula (I) or a pharmaceutically acceptable salt thereof.

In another embodiment the present invention provides a method of limiting tumourigenesis in a human or animal comprising administering to said human or animal a therapeutically effective amount of a compound of formula (I) or a pharmaceutically acceptable salt thereof.

In a further embodiment the present invention provides a method of inhibiting CHK1 kinase comprising administering to an animal or human in need of said inhibiting a therapeutically effective amount of a compound of formula (I) or a pharmaceutically acceptable salt thereof.

In a further embodiment the present invention provides a method of inhibiting a Pak kinase (for example a Pak1, Pak2 or Pak4 kinase) comprising administering to an animal or human in need of said inhibiting a therapeutically effective amount of a compound of formula (I) or a pharmaceutically acceptable salt thereof.

In a further embodiment the present invention provides a method of inhibiting PDK1 kinase comprising administering to an animal or human in need of said inhibiting a therapeutically effective amount of a compound of formula (I) or a pharmaceutically acceptable salt thereof.

In another embodiment the present invention provides a method of treatment of a human or animal suffering from cancer comprising administering to said human or animal a therapeutically effective amount of a compound of formula (I) or a pharmaceutically acceptable salt thereof.

In further embodiment the present invention provides a method of prophylaxis treatment of cancer comprising administering to a human or animal in need of such treatment a therapeutically effective amount of a compound of formula (I) or a pharmaceutically acceptable salt thereof.

In another embodiment the present invention provides a method of treatment of a human or animal suffering from a neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system, and other tumor types such as melanoma, multiple myeloma, fibrosarcoma and osteosarcoma, and malignant brain tumors.

In another embodiment the present invention provides a method of treatment of a human or animal suffering from a proliferative disease such as autoimmune, inflammatory, neurological, and cardiovascular diseases comprising administering to said human or animal a therapeutically effective amount of a compound of formula (I) or a pharmaceutically acceptable salt thereof.

One embodiment the of present invention provides a method of treating cancer by administering to a human or animal a compound of formula (I) or a pharmaceutically acceptable salt thereof and an anti-tumor agent.

One embodiment of the present invention provides a method of treating cancer by administering to a human or animal a compound of formula (I) or a pharmaceutically acceptable salt thereof and a DNA damaging agent.

One embodiment of the present invention provides a method for the treatment of infections associated with cancer comprising administering to a human or animal in need of such treatment a therapeutically effective amount of a compound of formula (I) or a pharmaceutically acceptable salt thereof.

A further embodiment of the present invention provides a method for the prophylaxis treatment of infections associated with cancer comprising administering to a human or animal in need of such treatment a therapeutically effective amount of a compound of formula (I) or a pharmaceutically acceptable salt thereof.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P.G.M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, maleic, tartaric, citric, ascorbic, palmitic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

“Stable compound” and “stable structure” are meant to indicate a compound that is sufficiently robust to survive isolation to a useful degree of purity from a reaction mixture, and formulation into an efficacious therapeutic agent.

The anti-cancer treatment defined herein may be applied as a sole therapy or may involve, in addition to the compound of the invention, conventional surgery and/or radiotherapy and/or chemotherapy. Such chemotherapy may include one or more of the following categories of anti-tumour agents:

(i) antiproliferative/antineoplastic drugs and combinations thereof, as used in medical oncology, such as alkylating agents or platinating (for example cis-platin, carboplatin, oxaliplatin, cyclophosphamide, nitrogen mustard, melphalan, chlorambucil, busulphan and nitrosoureas); antimetabolites (for example gemcitabine and fludarabine, as well as antifolates such as fluoropyrimidines like 5-fluorouracil and tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan, irinotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of infection is an amount sufficient to symptomatically relieve in a warm-blooded animal, particularly a human the symptoms of infection, to slow the progression of infection, or to reduce in patients with symptoms of infection the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. Examples of such acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Base salts include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic, physiologically acceptable salts are preferred, although other salts are also useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

In order to use a compound of the formula (I) or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compounds of formula (I) have been shown to inhibit checkpoint kinase activity in vitro. Inhibitors of checkpoint kinase have been shown to allow cells to progress inappropriately to the metaphase of mitosis leading to apoptosis of effected cells, and to therefore have anti-proliferative effects. Compounds of formula (I) have also been shown to inhibit Pak kinase and PDK1 kinase activity in vitro. Therefore it is believed that the compounds of formula (I) and their pharmaceutically acceptable salts may be used for the treatment of neoplastic disease as described above. In addition, compounds of formula (I) and their pharmaceutically acceptable salts are also expected to be useful for the treatment of other proliferative diseases and other diseases as described above. It is expected that the compounds of formula (I) would most likely be used in combination with a broad range of DNA damaging agents but could also be used as a single agent or in combination with another anti-tumour agent as described above

Generally, the compounds of formula (I) have been identified in one or more of the assays described below as having an IC₅₀ or EC₅₀ value of 100 micromolar or less. For example, in the Checkpoint Kinase 1 assay described below the compound of example 5 has and IC₅₀ value of 0.016 μM, example 16 has an IC₅₀ value of 0.55 μM and the compound of example 157 has an IC₅₀ value of 0.15 μM. By way of further examples, the compound of example 10 has an IC₅₀ value of 0.73 μM in the Pak 1 enzyme assay and an IC₅₀ value of 0.14 μM in the Pak 4 enzyme assay; the same compound has an IC₅₀ value of 0.35 μM in the PDK1 enzyme assay. The compound of example 14 has an IC₅₀ value of 0.60 μM in the Pak 1 enzyme assay and an IC₅₀ value of 0.10 μM in the Pak 4 enzyme assay; the same compound has an IC₅₀ value of 0.16 μM in the PDK1 enzyme assay.

### Assays to Measure Checkpoint Kinase 1 Inhibition and Effects

Checkpoint Kinase 1 Assay: This in vitro assay measures the inhibition of CHK1 kinase by compounds. The kinase domain is expressed in baculovirus and purified by the GST tag. Purified protein and biotinylated peptide substrate (Cdc25C) is then used in a 384 well automated Scintillation Proximity Assay (SPA). Specifically, peptide, enzyme and reaction buffer are mixed and aliquoted into a 384 well plate containing dilution series of compounds and controls. Cold and hot ATP are then added to initiate the reaction. After 2 hours, a SPA bead slurry, CsCl2 and EDTA are added to stop the reaction and capture the biotinylated peptide. Plates are then counted on a Topcount. Data is analyzed and IC₅₀s determined for individual compounds.

Abrogation Assay: This cellular assay measures the ability of CHK1 inhibitors to abrogate the DNA-damage induced G2/M checkpoint. Compounds active against the enzyme (<2 uM) are tested in the cellular assay. Briefly HT29 cells (colon cancer cell line, p53 null) are plated in 96 well plates on day 1. The following day, cells are treated with camptothecin for 2 hours to induce DNA damage. After 2 hours, camptothecin is removed and cells are treated for an additional 18 hours with test compound and nocodazole, a spindle poison that traps in cells in mitosis that abrogate the checkpoint. Cells are then fixed with formaldehyde, stained for the presence of phosphohistone H3, a specific marker for mitosis and labeled with Hoechst dye so that cell number can be measured. Plates are scanned using the Mitotic Index protocol on the Array Scan (Cellomics). As a positive control for abrogation, 4 mM caffeine is used. Compounds are tested in a 12-point dose response in triplicate. Data is analyzed and EC₅₀s determined for individual compounds.

### Assays to Measure Pak Kinase Inhibition:

### (a) In Vitro Pak1 Enzyme Assay

The assay used Scintillation Proximity Assay (SPA) technology (Antonsson et al., Analytical Biochemistry, 1999, 267: 294-299) to determine the ability of test compounds to inhibit phosphorylation by recombinant Pak1. The full-length Pak1 protein is expressed in E. coli as a GST fusion and purified using the GST tag using standard purification techniques.

Test compounds were prepared as 10 mM stock solutions in DMSO and diluted into water as required to give a range of final assay concentrations. Aliquots (5 μl) of each compound dilution were placed into a well of a Matrix 384-well flat bottom white polystyrene plate (Catalogue No. 4316). A 20 μl mixture of recombinant purified Pak1 enzyme (30 nM), 3 μM biotinylated peptide substrate (Biotin-Ahx-Lys-Lys-Glu-Gln-Ser-Lys-Arg-Ser-Thr-Met-Val-Gly-Thr-Pro-Tyr-Trp-Met-Ala-Pro-Glu-NH₂; Bachem UK Ltd), adenosine triphosphate (ATP; 3 μM), ³³P-labelled adenosine triphosphate (³³P-ATP; 33 nCi/well) and a buffer solution [comprising Tris-HCl pH7.5 buffer (50 mM), EGTA (0.1 mM), bovine serum albumin (0.1 mg/ml), dithiothreitol (DTT; 5 mM) and magnesium acetate (10 mM)] was incubated at ambient temperature for 120 minutes.

Control wells that produced a maximum signal corresponding to maximum enzyme activity were created by using 5% DMSO instead of test compound. Control wells that produced a minimum signal corresponding to fully inhibited enzyme were created by adding EDTA (62.5 mM) in 5% DMSO instead of test compound. These assay solutions were also incubated for 120 minutes at ambient temperature.

Each reaction was stopped and the biotinylated peptide captured by the addition of 30 Ξl of a mixture of Streptavidin coated PVT SPA bead slurry (Amersham Biosciences, Catalogue No. RPQO205; 250 μg/well) in 50 mM Tris-HCl pH7.5 buffer containing 0.05% sodium azide followed by the addition of 30 μl of 2.83M Caesium chloride (final assay concentration of 1M). Plates are then left for 2 hours on the bench before being counted on a TopCount.

Radiolabelled phosphorylated biotinylated peptide is formed in situ as a result of Pak1 mediated phosphorylation. The SPA beads contain a scintillant that can be stimulated to emit light. This stimulation only occurs when a radiolabelled phosphorylated peptide is bound to the surface of the Streptavidin coated SPA bead causing the emission of blue light that can be measured on a scintillation counter. Accordingly, the presence of Pak1 kinase activity results in an assay signal. In the presence of an Pak1 kinase inhibitor, signal strength is reduced.

Pak1 enzyme inhibition for a given test compound was expressed as an IC₅₀ value.

### (b) In Vitro Pak2 Enzyme Assay

The assay used Scintillation Proximity Assay (SPA) technology (Antonsson et al., Analytical Biochemistry, 1999, 267: 294-299) to determine the ability of test compounds to inhibit phosphorylation by recombinant Pak2. N-terminal and C-terminal His₆ tagged full-length Pak2 protein was expressed in E. coli and purified using Ni^{2+/}NTA-agarose.

Test compounds were prepared as 10 mM stock solutions in DMSO and diluted into water as required to give a range of final assay concentrations. Aliquots (5 μl) of each compound dilution were placed into a well of a Matrix 384-well flat bottom white polystyrene plate (Catalogue No. 4316). A 20 μl mixture of recombinant purified Pak2 enzyme (15 ng), 1 μM biotinylated peptide substrate (Biotin-Ahx-Lys-Lys-Glu-Gln-Ser-Lys-Arg-Ser-Thr-Met-Val-Gly-Thr-Pro-Tyr-Trp-Met-Ala-Pro-Glu-NH₂; Bachem UK Ltd), adenosine triphosphate (ATP; 2 μM), ³³P-labelled adenosine triphosphate (³³P-ATP; 33 nCi/well) and a buffer solution [comprising Tris-HCl pH7.5 buffer (50 mM), EGTA (0.1 nM), bovine serum albumin (0.1 mg/ml), dithiothreitol (DTT; 5 nM) and magnesium acetate (10 mM)] was incubated at ambient temperature for 120 minutes.

Control wells that produced a maximum signal corresponding to maximum enzyme activity were created by using 5% DMSO instead of test compound. Control wells that produced a minimum signal corresponding to fully inhibited enzyme were created by adding EDTA (62.5 mM) in 5% DMSO instead of test compound. These assay solutions were also incubated for 120 minutes at ambient temperature.

Each reaction was stopped and the biotinylated peptide captured by the addition of 30 μl of a mixture of Streptavidin coated PVT SPA bead slurry (Amersham Biosciences, Catalogue No. RPQ0205; 250 μg/well) in 50 mM Tris-HCl pH7.5 buffer containing 0.05% sodium azide followed by the addition of 30 μl of 2.83M Caesium chloride (final assay concentration of 1M). Plates are then left for 2 hours on the bench before being counted on a TopCount.

Radiolabelled phosphorylated biotinylated peptide is formed in situ as a result of Pak2 mediated phosphorylation. The SPA beads contain a scintillant that can be stimulated to emit light. This stimulation only occurs when a radiolabelled phosphorylated peptide is bound to the surface of the Streptavidin coated SPA bead causing the emission of blue light that can be measured on a scintillation counter. Accordingly, the presence of Pak2 kinase activity results in an assay signal. In the presence of a Pak2 kinase inhibitor, signal strength is reduced.

Pak2 enzyme inhibition for a given test compound was expressed as an IC₅₀ value.

### (c) In Vitro Pak4 Enzyme Assay

The assay used Scintillation Proximity Assay (SPA) technology (Antonsson et al., Analytical Biochemistry, 1999, 267: 294-299) to determine the ability of test compounds to inhibit phosphorylation by recombinant Pak4. The kinase domain of Pak4 (amino acids 291 to 591) is expressed in E. coli as a GST fusion and purified using the GST tag using standard purification techniques.

Test compounds were prepared as 10 mM stock solutions in DMSO and diluted into water as required to give a range of final assay concentrations. Aliquots (5 μl) of each compound dilution were placed into a well of a Matrix 384-well flat bottom white polystyrene plate (Catalogue No. 4316). A 20 μl mixture of recombinant purified Pak4 enzyme (10 nM), 1 μM biotinylated peptide substrate (Biotin-Ahx-Lys-Lys-Glu-Val-Pro-Arg-Arg-Lys-Ser-Leu-Val-Gly-Thr-Pro-Tyr-Trp-Met-Ala-Pro-Glu-NH₂; Bachem UK Ltd), adenosine triphosphate (ATP; 2 μM), ³³P-labelled adenosine triphosphate (³³P-ATP; 33 nCi/well) and a buffer solution [comprising Tris-HCl pH7.5 buffer (50 mM), EGTA (0.1 mM), bovine serum albumin (0.1 mg/ml), dithiothreitol (DTT; 5 mM) and magnesium acetate (10 mM)] was incubated at ambient temperature for 120 minutes.

Control wells that produced a maximum signal corresponding to maximum enzyme activity were created by using 5% DMSO instead of test compound. Control wells that produced a minimum signal corresponding to fully inhibited enzyme were created by adding EDTA (62.5 mM) in 5% DMSO instead of test compound. These assay solutions were also incubated for 120 minutes at ambient temperature.

Each reaction was stopped and the biotinylated peptide captured by the addition of 30 μl of a mixture of Streptavidin coated PVT SPA bead slurry (Amersham Biosciences, Catalogue No. RPQ0205; 250 μg/well) in 50 mM Tris-HCl pH7.5 buffer containing 0.05% sodium azide followed by the addition of 30 μl of 2.83M Caesium chloride (final assay concentration of 1M). Plates are then left for 2 hours on the bench before being counted on a TopCount.

Radiolabelled phosphorylated biotinylated peptide is formed in situ as a result of Pak4 mediated phosphorylation. The SPA beads contain a scintillant that can be stimulated to emit light. This stimulation only occurs when a radiolabelled phosphorylated peptide is bound to the surface of the Streptavidin coated SPA bead causing the emission of blue light that can be measured on a scintillation counter. Accordingly, the presence of Pak4 kinase activity results in an assay signal. In the presence of an Pak4 kinase inhibitor, signal strength is reduced.

Pak4 enzyme inhibition for a given test compound was expressed as an IC₅₀ value. Typical activity of the compounds is in the range 10 nM to 20 μM.

Assays to Measure PDK1 Kinase Inhibition:

### (a) PDK1 Enzyme Assay:

The assay utilised Alphascreen technology (Ullman, E F, et al. Proc. Natl. Acad. Sci. USA, Vol. 91, pp. 5426-5430, 1994) to measure the ability of compounds to inhibit PDK1 enzyme activity. 6H is tagged PDK1 enzyme was expressed in insect cells and purified using NiNTA beads and conventional protein purification methodology.

Test compounds were prepared as 10 mM stock solutions in DMSO and diluted into water as required to give a range of final assay concentrations. 2 μl aliquots of compounds were dispensed into Greiner Bio-One low volume 384 well plates (Catalogue no. 784075). For the activity assay, to each well was added 5 μl of a mixture of 48 nM native peptide (Biotin-Ahx-Ile-Lys-Asp-Gly-Ala-Thr-Met-Lys-Thr-Phe-Cys-Gly-Thr-Pro-Glu-Tyr-Leu-Ala-Pro-Glu-Val-Arg-Arg-Glu-Pro-Arg-Ile-Leu-Ser-Glu-Glu-Glu-Gln-Glu-Met-Phe-Arg-Asp-Phe-Asp-Tyr-Ile-Ala-Asp-Trp-NH₂, Bachem UK Ltd) and 12 μM adenosine triphosphate (ATP) in reaction buffer comprising Tris-HCl pH7.4 (60 mM), magnesium acetate (12 mM), EGTA (120 μM), DTT (1.2 mM) and bovine serum albumin (0.12 mg/ml). The reaction was started by addition of 5 μl of a freshly prepared solution containing 20 ng/ml of purified recombinant PDK1 protein in reaction buffer and incubated at room temperature for 45 minutes.

Each reaction was stopped by addition of 5 μl of a solution containing Tris-HCl pH7.4 (50 mM), bovine serum albumin (1 mg/ml), EDTA (90 mM), anti-phospho Akt T308 antibody, R&D Systems, Catalogue no. RF8871, (200 ng/ml), Alphascreen streptavidin donor bead, Perkin Elmer Catalogue no. 6760002B (30 μg/ml) and Alphascreen Protein A acceptor bead, Perkin Elmer Catalogue no. 6760137R (30 μg/ml). Plates were then sealed and incubated overnight in low light conditions before being read on an Envision plate reader (Perkin Elmer).

Compounds were also tested in an artefact assay using similar conditions to the activity assay but in the presence of 2 nM phosphorylated peptide [Biotin-Ahx-Ile-Lys-Asp-Gly-Ala-Thr-Met-Lys-(p)Thr-Phe-Cys-Gly-Thr-Pro-Glu-Tyr-Leu-Ala-Pro-Glu-Val-Arg-Arg-Glu-Pro-Arg-Ile-Leu-Ser-Glu-Glu-Glu-Gln-Glu-Met-Phe-Arg-Asp-Phe-Asp-Tyr-Ile-Ala-Asp-Trp-NH₂, Bachem UK Ltd) and 18 nM native peptide.

Control wells that produced a maximum signal corresponding to maximum enzyme activity were created by using 6% DMSO instead of test compound. Control wells that produced a minimum signal were created by adding EDTA (0.5M) for the activity assay or by addition of 1.008 mM Coomassie blue for the artefact assay instead of test compound.

Phosphorylated biotinylated peptide is formed by the activity of PDK1 in the activity assay and is subsequently bound by the anti-phospho Akt T308 antibody. This complex is then captured by both the streptavidin donor bead via its interaction with biotin, and the Protein A acceptor bead via its interaction with the antibody. The proximity of the donor and acceptor beads now enables transfer of singlet oxygen from the donor bead by excitation at 680 nm to the acceptor bead causing emission at 520-620 nm. The strength of signal is proportion to the activity of the PDK1 enzyme within the linear range of the assay. Hence the presence of inhibitors of PDK1 activity will diminish the emission at 520-620 nm.

PDK1 inhibitor activity was reported as an IC₅₀ value from duplicate measurements.

### (b) PDK1 Cell Assay:

As part of the PI3K pathway, Akt is a PIF pocket independent substrate of PDK and phosphorylation of T308 on Akt1 provides a direct measure of cellular PDK1 activity. Cell types with mutations of the PI3K pathway (eg PTEN, PI3K) can be employed in an assay to either avoid the need to stimulate or maximise on pathway flux. The cell assay utilises a phospho specific antibody to detect Akt1 phosphorylation on T308.

Breast adenocarcinoma cell line MDA-MB-468 cells (PTEN null) were seeded in 96 well plates (Packard Viewplates, Perkin Elmer Catalogue no. 1450-573) at a density of 10⁴ cells per well in 90 μl and grown overnight at 37° C., 5% CO₂. Test compounds were prepared as 10 mM stock solutions in DMSO and diluted into cell media as required to give a range of final assay concentrations. 10 ul of compound was then added to each well and cells incubated for 2 hrs at 37° C., 5% CO₂. Cells were then fixed by adding 20 μl 10% formaldehyde in phosphate buffered saline (PBS) to each well and incubating for 20 minutes at room temperature. Following removal of media and fix, cells were washed with 100 ul PBS, 0.05% polysorbate and then permeabilised by addition of 100 μl PBS, 0.5% Tween 20 and incubated at room temperature for 10 minutes. After removal of permeabilisation buffer, cells were stained for presence of phosphoAkt1 T308, phosphoAkt2 T309 and phosphoAkt3 T305.

Briefly cells were incubated at room temperature in 100 μl of blocking buffer (PBS, 0.05% Tween 20, 5% BSA) for 1 hr and then stained overnight at 4° C. with 40 μl per well of solution of anti-phospho Akt T308 antibody (Cell Signalling Technologies, Catalogue no. 4056) diluted 1/1000 in blocking buffer. Following 3 washes with 250 μl of PBS, 0.05% Tween 20, cells were stained for 1 hr at room temperature with 40 ul of a solution containing a 1/1000 dilution of goat anti-rabbit IgG (H+L)/Alexa Fluor 488 conjugate (Molecular Probes, Catalogue no. A11008) in blocking buffer. After a further 3 washes in 250 μl of PBS, 0.05% Tween 20, 1001 of PBS, 0.05% polysorbate, 1 μM propidium iodide was added and the plates read on an Acumen Explorer plate reader (TTP Labtech).

All measurements were carried out in duplicate and quantified signal used to estimate IC50 values for compounds. Effect on cell number was monitored using the propidium iodide staining.

### Synthesis

The compounds of the present invention can be prepared in a number of ways well known to one skilled in the art of organic synthesis. More specifically, the novel compounds of this invention may be prepared using the reactions and techniques described herein. In the description of the synthetic methods described below, it is to be understood that all proposed reaction conditions, including choice of solvent, reaction atmosphere, reaction temperature, duration of the experiment and workup procedures, are chosen to be the conditions standard for that reaction. It is understood by one skilled in the art of organic synthesis that the functionality present on various portions of the molecule must be compatible with the reagents and reactions proposed. Such restrictions to the substituents, which are not compatible with the reaction conditions, will be apparent to one skilled in the art and alternate methods must then be used.

Unless otherwise stated, the starting materials for the examples contained herein are either commercially available or are readily prepared by standard methods from known materials. General procedures for synthesizing the compounds of the invention are as follows: Compounds of Formula (I) can be synthesized from the general synthetic methods described below in Schemes 1-10. A general method for the synthesis of thienopyridine compounds of Formula (I) is described in Scheme 1. Nitriles substituted with aryl groups can be condensed with glyoxylic acid in a Knovenagel type manner to give unsaturated carboxylates. Generation of the acid chloride followed by reaction with sodium azide yields an acyl azide. Curtius rearrangement of the acyl azide followed by electrophilic cyclization of the thiophene ring gives the thienopyridone using very high temperatures. The 5-position of the thiophene ring can be then selectively brominated or iodinated by choice of reaction with N-bromo- or iodo-succinimide. Dehydration and aromatization using phosphorus oxychloride yields a chloropyridine intermediate, which can undergo nucleophilic displacement by reaction of an amine with potassium carbonate in NMP. Alternatively, the chloropyridine intermediate can react with oxygen or sulfur nucleophiles to give the corresponding aryl ethers or sulfides (Scheme 2). The resultant bromo- or iodo-thienopyridines can react in Pd-mediated Suzuki reactions with boronic acids or esters under standard coupling conditions. The desired thienopyridine carboxamides can be finally generated by partial hydrolysis of the nitrile using concentrated hydrochloric acid or PPA. A modification to the synthesis, shown in Scheme 3, allows for hydrolysis prior to Suzuki Coupling.

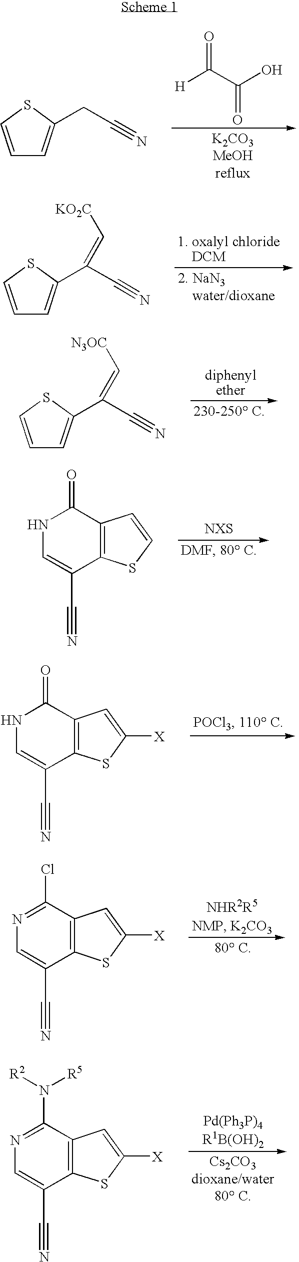

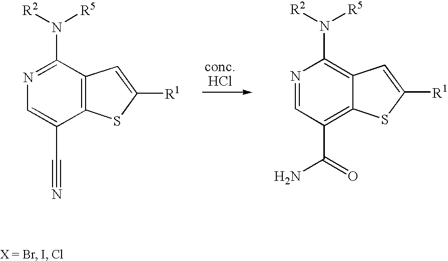

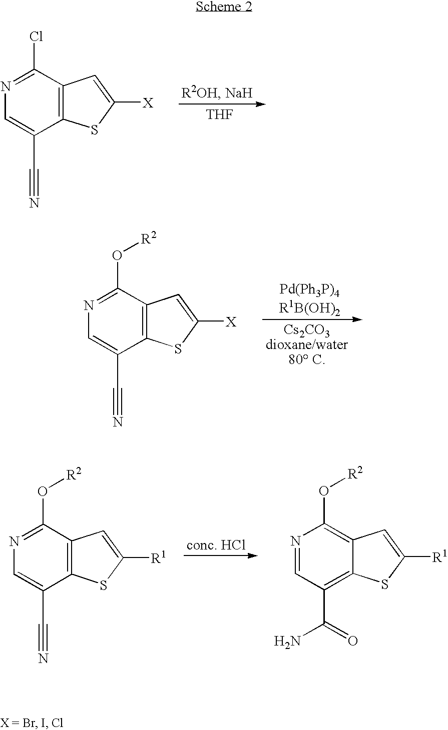

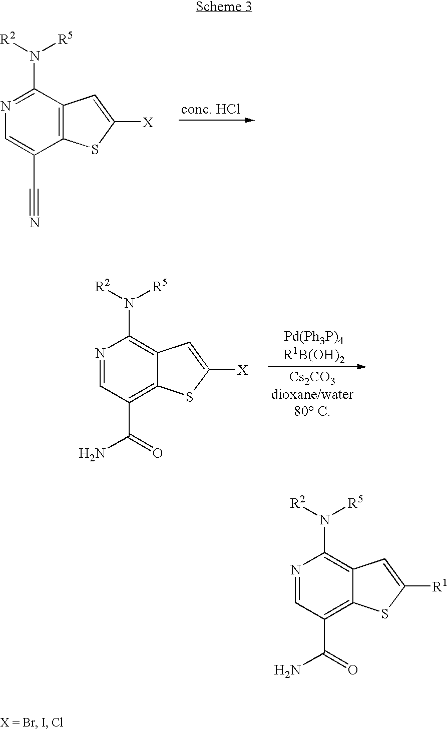

The bromo, chloro or iodo-thienopyridines from Schemes 1-3 can also be used in other Pd-mediated coupling reactions such as Stille Couplings with arylstannanes (Scheme 4), Sonogashiri Couplings with alkynes (Scheme 5), and Buchwald Aminations (Scheme 6) to form compounds of Formula (I).

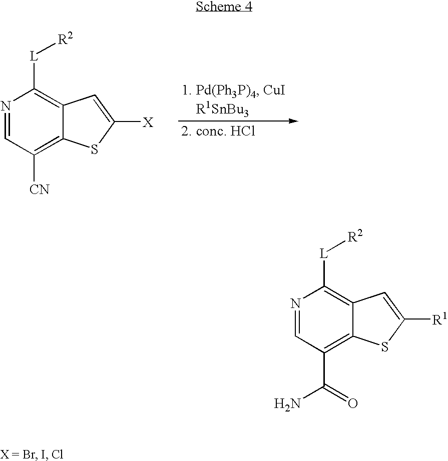

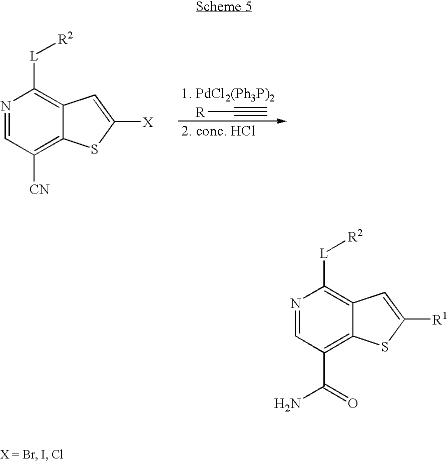

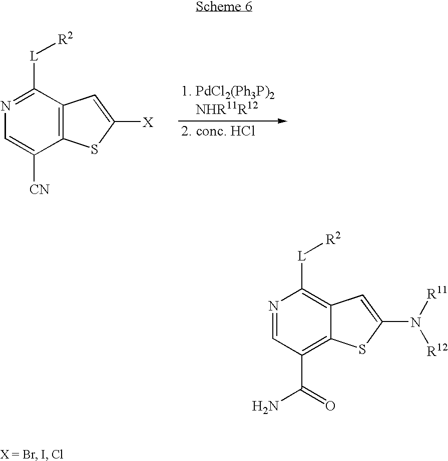

Other heterocyclic compounds of Formula (I) can be generated using the alternate synthetic route outlined in Scheme 7. Heteroaryl aldehydes can undergo Aldol type condensations with malonic acid to give unsaturated carboxylic acids. Similar to Scheme 1, generation of the acid chloride followed by acyl azide formation and cyclization via the Curtius isocyanate intermediate furnishes the heterocyclic 5-6 fused pyridine. Bromination of the pyridine ring followed by displacement with copper cyanide furnishes the nitrile pyridine. Reaction as before with phosphorous oxychloride and amine displacement provides the nitrile precursor. The target compounds of Formula (I) as then formed by hydrolysis of the nitrile to the desired carboxamide.

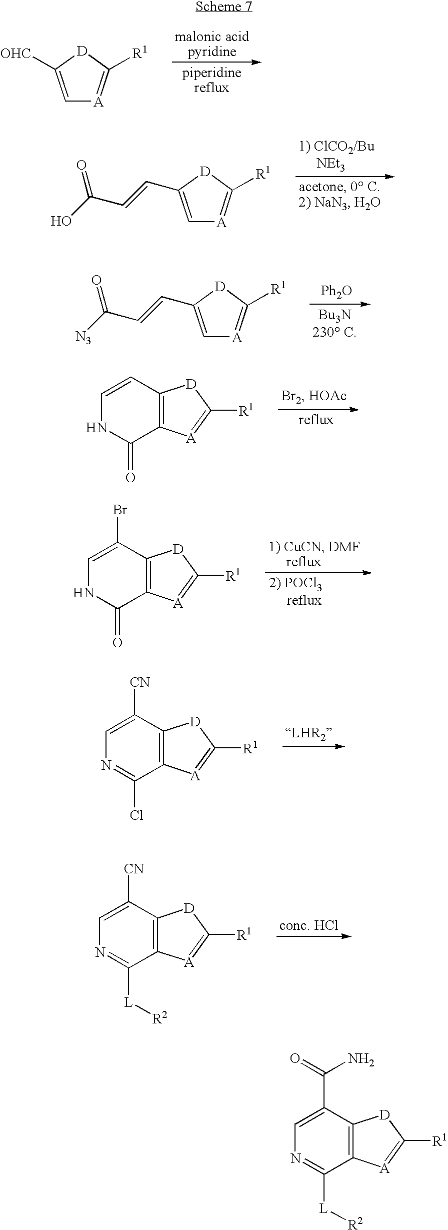

Compounds of Formula (I) where substituted amides are desired can be synthesized following the steps outlined in Scheme 8. An aminonitrile generated from any of the above Schemes 1-7 can be hydrolyzed completely to the carboxylic acid by the alternate use of aqueous 6N hydrochloric acid in place of the concentrated variety. The acid can then be coupled to an amine using any standard amide formation methods such as reaction with mixed anhydrides of the acid or the use of amide coupling/dehydrating agents such as, but not limited to, 1-(3-dimethylaminopropyl)-3-ethylcarbodiimide hydrochloride (EDCI), O-(1H-benzotriazol-1-yl)-N,N,N′,N′-tetramethyluronium hexafluorophosphate (HBTU), or benzotriazol-1-yloxytris(dimethylamino)phosphonium hexafluorophosphate (BOP).

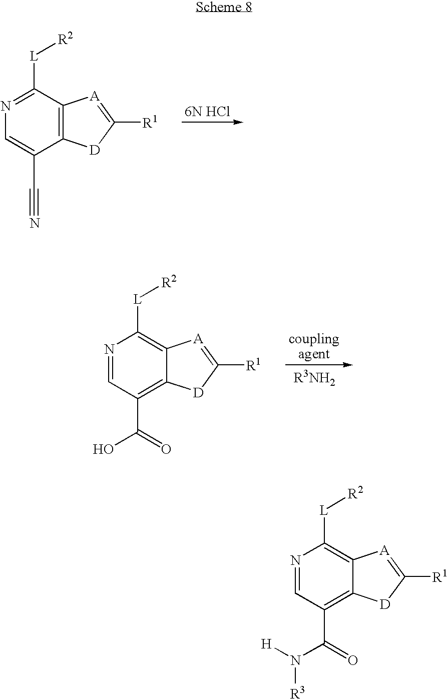

Non-pyridyl compounds of Formula (I) can be synthesized using the synthetic transformations described in Schemes 9 and 10. Shown in Scheme 9, 3-nitro anthranilate derivatives can be esterified using MeOH and anhydrous hydrochloric acid under reflux. The amino ester can then undergo a one-pot transformation using potassium tert-butoxide in DMSO which involves generation of an enamine by reaction with an aryl ketone, deprotonation, nucleophilic aromatic substitution ortho to the activating nitro group, followed by oxidation to the indole intermediate. The carboxylic acid product of this step can be converted to amides by formation of the mixed anhydride with a chloroformate, followed by reaction with an amine. Hydrogenation of the nitro group yields the aniline derivative that can be converted to indole compounds of Formula (I) by reductive amination with ketones or aldehydes. Alternatively, the aniline can be converted to halide derivatives through diazotization methodology and then converted into compounds of Formula (I) by Pd or Cu mediated displacements with amines or alcohols.

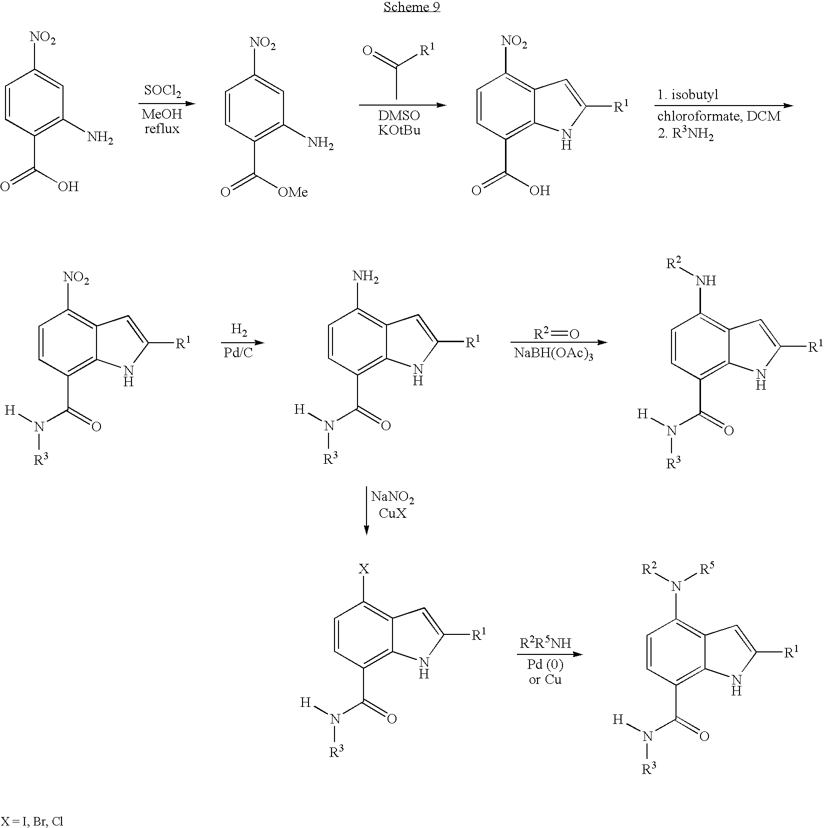

Another route to non-pyridyl compounds of Formula (I) is outlined in Scheme 10. Nucleophilic aromatic substitution of fluorine with amines, alcohols, or thiols followed by generation of an aniline and subsequent intramolecular electrophilic aromatic substitution of the acetal with strong acid can give the various 5-6 fused systems. The resultant aniline can be elaborated into non-pyridyl compounds such as benzothiophenes or benzofurans of Formula (I) using the previously described transformations in Schemes 1-9.

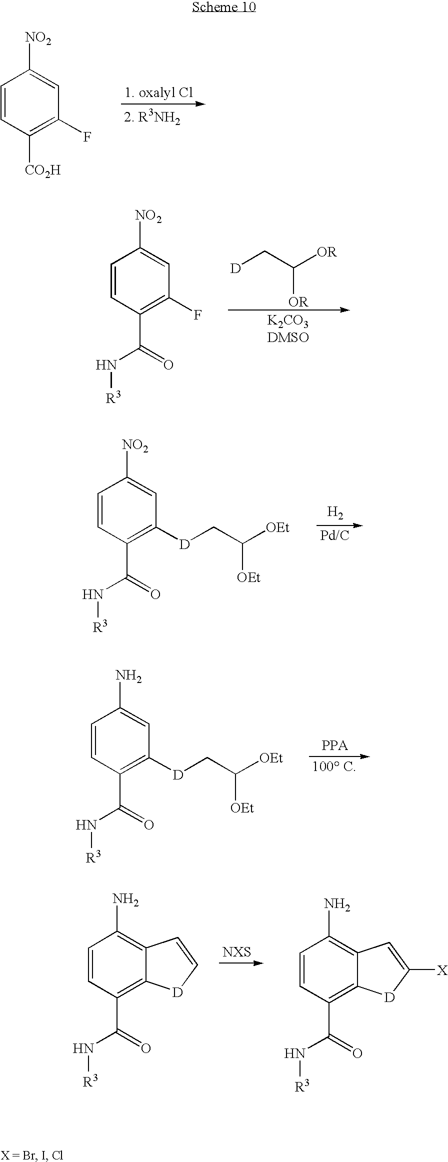

### EXAMPLES

The invention will now be further described with reference to the following illustrative examples in which, unless stated otherwise:
(i) temperatures are given in degrees Celsius (° C.); operations are carried out at room temperature or ambient temperature, that is, in a range of 18-25° C., unless otherwise stated;
(ii) solutions are dried over anhydrous sodium sulphate or magnesium sulphate; evaporation organic of organic solvent is carried out using a rotary evaporator under reduced pressure (4.5-30 mmHg) with a bath temperature of up to 60 C;
(iii) chromatography means flash chromatography on silica gel; thin layer chromatography (TLC) is carried out on silica gel plates;
(iv) in general, the course of reactions are followed by TLC or liquid chromatography/mass spectroscopy (LC/MS) and reaction times are given for illustration only;
(v) final products have satisfactory proton nuclear magnetic resonance (NMR) spectra and/or mass spectra data;
(vi) yields are given for illustration only and are not necessarily those which can be obtained by diligent process development; preparations are repeated if more material is required;
(vii) when given, nuclear magnetic resonance (NMR) data is in the form of delta (δ) values for major diagnostic protons, given in part per million (ppm) relative to tetramethylsilane (TMS) as an internal standard, determined at 300 MHz in d₆-DMSO unless otherwise stated;
(viii) chemical symbols have their usual meanings;
(ix) solvent ratio is given in volume:volume (v/v) terms;
(x) Rochelle's Salt is sodium potassium tartrate;
(xi) Hunig's Base is diisopropylethylamine (DIEA);
(xii) Purification of the compounds are carried out using one or more of the following methods:

a) flash chromatography on regular silica gel;

b) flash chromatography on silica gel using an MPLC separation system: prepacked normal phase flash column cartridge, flow rate, 30-40 ml/min;

c) Preparatory HPLC system using a reverse-phase C18 column, 100×20 mm, 5 uM (or larger) and eluting with combinations of water (0.1% TFA) and MeCN (0.1% TFA) as the mobile phase;

d) Chiral Preparatory HPLC system using a chiral column, e.g. Diacel® column (AD, OD, AS, and/or OJ stationary phases), 250×20 mm, 10 uM (or larger) and eluting with combinations of hexane, isopropanol, EtOH, and/or MeOH with 0.1% diisopropylethylamine as the mobile phase; and

(xiii) the following abbreviations have been used:
CIV concentrated in vacuo;
RT and rt room temperature;
BOC tert-butoxycarbonyl;
BOP benzotriazol-1-yloxytris(dimethylamino)phosphonium hexafluorophosphate;
Bu₃N tributylamine;
CBZ benzyloxycarbonyl;
DMF N,N-dimethylformamide;
DMSO dimethylsulfoxide;
NMP N-methyl-2-pyrrolidinone;
EtOAc ethyl acetate;
ether diethyl ether;
EtOH ethanol;
THF tetrahydrofuran;
MeOH methanol;
MeCN acetonitrile;
PPA polyphosphoric acid;
TFA trifluoracetic acid; and
TEA triethylamine.

Most of the compounds prepared in the Examples below were isolated as the hydrochloride salts which will be apparent to one of skill in the art based on the procedures used to prepare the compounds and as evidenced by the NMR data.

### Example 1

### 2-phenyl-4-[(3S)-piperidin-3-ylamino]thieno[3,2-c]pyridine-7-carboxamide

### Step 1:

(2Z)-3-cyano-3-(2-thienyl)acrylic acid. To a stirred solution of 2-thienylacetonitrile (24.8 g, 0.20 mol) in MeOH (300 mL) is added glyoxylic acid monohydrate (18.5 g, 0.20 mol) and potassium carbonate (25.5 g, 0.20 mol). The reaction slurry is placed under a nitrogen atmosphere and heated to reflux. After 2 h the reaction mixture is cooled to rt and the product is obtained by filtration. The filter cake is washed with a large amount of MeOH and then dried in a vacuum oven overnight to give 43.1 g (99%) of the title compound as a white crystalline potassium salt. ¹H NMR δ 7.95 (d, 3H), 7.75 (d, 1H), 7.30 (dd, 1H), 7.05 (s, 1H), 3.0-4.0 (br s, 1H). LCMS (ES, M+H=180, M−H=178).

### Step 2:

(2Z)-3-cyano-3-(2-thienyl)acryloyl chloride. To a stirred solution of oxalyl chloride (2.6 mL, 30 mmol) in 10 mL of CH₂Cl₂ is added a solution of (2Z)-3-cyano-3-(2-thienyl)acrylic acid potassium salt (2.2 g, 12.3 mmol) dissolved in 20 mL of CH₂Cl₂. An additional amount of CH₂Cl₂ is added until the viscous heterogeneous reaction mixture can be stirred easily. The reaction is stirred for about 1 h at rt. The solids are removed by filtration and washed with generous amounts of CH₂Cl₂. The filtrate and ishes are combined and concentrated in vacuo to yield 2.0 g of the title compound that is used in the next step.

### Step 3:

(2Z)-3-cyano-3-(2-thienyl)acryloyl azide. To a rapidly stirred suspension of sodium azide (2.0 g, 30 mmol) in a 50:50 mixture of dioxane/water (20 mL) at 0° C. is added a solution of (2Z)-3-cyano-3-(2-thienyl)acryloyl chloride (2.0 g, 12.3 mmol) dissolved in 10 mL of dioxane. The reaction is stirred for 30 min. at 0° C. and then allowed to reach rt after 1-2 h further stirring. The reaction is then added to ˜100 mL of water. The precipitate formed is filtered, washed with water, and dried in the vacuum oven overnight to give the title azide as a white solid (2.0 g), which is used in the next step without further purification.

### Step 4:

4-oxo-4,5-dihydrothieno[3,2-c]pyridine-7-carbonitrile. A mixture of diphenyl ether (260 mL) and Bu₃N (53 mL) is heated to 210° C. under a stream of nitrogen. A slurry of (2Z)-3-cyano-3-(2-thienyl)acryloyl azide (15.0 g, 73.5 mmol) in CH₂Cl₂ (30 mL) is added dropwise over 2 h (vigorous evolution of N₂ gas). After the addition is complete the reaction is stirred at 210° C. for a further 10 min, then the reaction is allowed to cool to rt, then in an ice bath. Hexanes (500 mL) is added and the precipitate is filtered off under suction, washing with copious quantities of hexanes. The obtained solid is dried in a vacuum oven overnight (without heating) to obtain the title compound as a pale brown solid (9.89 g, 76%). ¹H NMR δ12.4 (br s, 1H), 8.29 (d, 1H), 7.82 (d, 1H), 7.58 (d, 1H). LCMS (ES, M+H=177, M−H=175).

### Step 5:

2-bromo-4-oxo-4,5-dihydrothieno[3,2-c]pyridine-7-carbonitrile. A solution of 4-oxo-4,5-dihydrothieno[3,2-c]pyridine-7-carbonitrile (0.8 g, 4.5 mmol) in a 50:50 mixture of DMF/Acetic Acid (20 mL) is charged with N-bromosuccinimide (1.6 g, 9 mmol). The dark reaction mixture is heated to 80° C. for 12 h. After cooling to rt, the reaction is added to ˜100 mL of water while stirring. The pH of the cloudy solution is adjusted to 9-10 with sat. NaHCO₃. The product is obtained by filtration, washed with water, and is dried in a vacuum oven (1.1 g, 100%). ¹H NMR δ 12.6 (br s, 1H), 8.38 (d, 1H), 7.77 (s, 1H). LCMS (ES, M+H=255, M−H=253).

### Step 6:

2-bromo-4-chlorothieno[3,2-c]pyridine-7-carbonitrile. A solution of 2-bromo-4-oxo-4,5-dihydrothieno[3,2-c]pyridine-7-carbonitrile (1.1 g, 4.5 mmol) dissolved in POCl₃ (10 mL) is heated to reflux overnight. After cooling to rt, the reaction is concentrated to dryness under vacuum. The solids are slowly and carefully suspended in 50-100 mL of water. The product is obtained by filtration, followed by washing with water, saturated NaHCO₃, water, and drying in a vacuum oven (1.0 g, 83%). ¹H NMR δ 9.10 (s, 1H), 8.20 (s, 1H). LCMS (ES, M+H=275).

### Step 7:

tert-butyl (3S)-3-[(7-cyano-2-bromothieno[3,2-c]pyridin-4-yl)amino]piperidine-1-carboxylate. To a stirred solution of 2-bromo-4-chlorothieno[3,2-c]pyridine-7-carbonitrile (0.48 g, 1.76 mmol) and tert-butyl (3S)-3-aminopiperidine-1-carboxylate (0.40 g, 2.0 mmol) in NMP (5 mL) is added potassium carbonate (0.5 g, 3.52 mmol). The heterogeneous mixture is heated to 80° C. for 2 h, cooled to rt, and then added to 50 mL of water. The product (880 mg) is isolated by filtration and dried. The title compound is further purified using MPLC (SiO₂; 30-50% EtOAc/Hexanes gradient) to give 0.54 g, 70% as a light yellow crystalline solid. ¹H NMR δ 8.36 (s, 1H), 8.08 (s, 1H), 7.68 (m, 1H), 4.02 (m, 1H), 3.74 (m, 1H), 3.50 (m, 1H), 2.70-3.20 (m, 2H), 1.91 (m, 1H), 1.74 (m, 1H), 1.54 (m, 1H), 1.30-1.45 (m, 1H), 1.21 (s, 9H). LCMS (ES, M+H=437, 439; M−H, 435, 437).

### Step 8:

tert-butyl (3S)-3-{[7-cyano-2-(phenyl)thieno[3,2-c]pyridin-4-yl]amino}piperidine-1-carboxylate. A mixture of tert-butyl (3S)-3-[(7-cyano-2-bromothieno[3,2-c]pyridin-4-yl)amino]piperidine-1-carboxylate (0.18 g. 0.41 mmol), phenylboronic acid (0.076 g, 0.62 mmol), palladium(0)tetrakis triphenylphosphine (Pd(PPh₃)₄), (0.10 g, 0.062 mmol), and cesium carbonate (0.41 g, 1.25 mmol), are dissolved in water (1 mL), and dioxane (3 mL). This reaction mixture is stirred at 80° C. for 1 h under a nitrogen atmosphere, and then allowed to cool to rt. The water is removed with a pipette and dioxane is removed under vacuum. The residue is purified by MPLC (SiO₂; 30-60% EtOAc/Hexanes) gave the title compound (140 mg, 78%). ¹H NMR δ 8.41 (s, 1H), 8.34 (br s, 1H), 7.74 (s, 1H), 7.73 (d, 2H), 7.52 (dd, 2H), 7.42 (dd, 1H), 4.12 (m, 1H), 3.81 (m, 1H), 3.60 (m, 1H), 2.6-3.2 (m, 2H), 2.01 (m, 1H), 1.82 (m, 1H), 1.62 (m, 1H), 1.40-1.50 (m, 1H), 1.24 (s, 9H). LCMS (ES, M+H=435; M−H, 433).

### Step 9:

2-phenyl-4-[(3S)-piperidin-3-ylamino]thieno[3,2-c]pyridine-7-carboxamide. A solution of tert-butyl (3S)-3-{[7-cyano-2-(phenyl)thieno[3,2-c]pyridin-4-yl]amino}piperidine 1-carboxylate (80 mg, 0.18 mmol) and 12N HCl (conc., 4 mL) is stirred for 24 hours. Water (10-20 mL) is added and the pH of the solution is adjusted to 10-11 with sat. NaHCO₃. The material is isolated by filtration and is washed with a small amount of cold water. The material is dried and then purified by MPLC (SiO₂; NH₄OH/MeOH/CH₂Cl₂; 2:10:88) to give the title compound (24 mg, 38%). ¹H NMR δ 8.58 (s, 1H), 8.27 (s, 1H), 7.98 (br s, 1H), 7.79 (d, 2H), 7.55 (dd, 2H), 7.43 (dd, 1H), 7.34 (br s, 1H), 7.25 (d, 2H), 4.22 (m, 1H), 3.21 (d, 1H), 2.91 (d, 1H), 2.48 (m, 2H), 2.05 (m, 1H), 1.76 (m, 1H), 1.55 (m, 2H). LCMS (ES, M+H=353).

Examples 2-51 are made in a similar fashion as example 1 using appropriate starting materials.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 2 | 4-[(3S)-piperidin-3- | 358.49 | 359 | 9.72 (s, 1H), 9.08 (s, 1H), 8.79 (s, 1H), |
| | ylamino]-2-(3- | | | 8.55 (s, 1H), 8.40 (s, 1H), 7.96 (s, 1H), |
| | thienyl)thieno[3,2- | | | 7.78 (t, 1H), 7.72 (s, 1H), 7.54 (d, 1H), |
| | c]pyridine-7-carboxamide | | | 4.63 (m, 1H), 3.19 (m, 2H), 3.02 (m, |
| | | | | 2H), 2.06 (m, 2H), 1.81 (m, 2H). |
| 3 | 2-(3-fluorophenyl)-4- | 370.45 | 371 | 8.59 (s, 1H) 8.34 (s, 1H) 7.98 (s, 1H) |
| | [(3S)-piperidin-3- | | | 7.51-7.64 (m, 3H), 7.37 (s, 1H) |
| | ylamino]thieno[3,2- | | | 7.21-7.30 (m, 3H) 4.22 (s, 1H) |
| | c]pyridine-7-carboxamide | | | 3.19 (d, 1H) 2.90 (d, 1H) 2.38-2.53 (m, 2H) |
| | | | | 2.04 (m, 1H) 1.75 (m, 1H) |
| | | | | 1.54 (m, 2H) |
| 4 | 2-(2,4-difluorophenyl)-4- | 388.44 | 389 | 9.61 (s, 1H), 9.01 (s, 1H), 8.80 (s, 1H), |
| | [(3S)-piperidin-3- | | | 8.59 (s, 1H), 8.42 (m, 1H), 7.92 (m, |
| | ylamino]thieno[3,2- | | | 1H), 7.67 (s, 1H), 7.52 (m, 1H), |
| | c]pyridine-7-carboxamide | | | 7.30 (m, 1H), 4.63 (m, 1H), 3.20 (m, 2H), |
| | | | | 2.99 (m, 2H), 2.08 (m, 2H), 1.80 (m, |
| | | | | 2H). |
| 5 | 2-(3-methoxyphenyl)-4- | 382.49 | 383 | 9.02 (s, 1H), 8.82 (s, 1H), 8.55 (s, 1H), |
| | [(3S)-piperidin-3- | | | 8.37 (s, 1H), 8.12 (s, 1H), 7.43 (t, 2H), |
| | ylamino]thieno[3,2- | | | 7.32 (m, 2H), 6.98 (d, 1H), 4.53 (m, |
| | c]pyridine-7-carboxamide | | | 1H), 3.86 (s, 3H), 3.21 (m, 2H), |
| | | | | 2.97 (m, 2H), 2.04 (m, 2H), 1.74 (m, 2H). |
| 6 | 2-(4-methylphenyl)-4- | 366.49 | 367 | 9.61 (s, 1H), 9.02 (s, 1H), 8.76 (s, 1H), |
| | [(3S)-piperidin-3- | | | 8.54 (s, 1H), 8.39 (s, 1H), 7.70 (d, 2H), |
| | ylamino]thieno[3,2- | | | 7.34 (d, 2H), 4.61 (m, 1H), 3.19 (m, |
| | c]pyridine-7-carboxamide | | | 2H), 2.36 (s, 3H), 2.07 (m, 2H), |
| | | | | 1.80 (m, 2H). |
| 7 | 2-(3,4-dimethoxyphenyl)- | 412.51 | 413 | 9.25 (s, 1H), 8.88 (s, 1H), 8.51 (s, 1H), |
| | 4-[(3S)-piperidin-3- | | | 8.45 (s, 1H), 8.21 (s, 1H), 7.55 (s, 1H), |
| | ylamino]thieno[3,2- | | | 7.32 (d, 2H), 7.10 (d, 1H), 4.55 (m, |
| | c]pyridine-7-carboxamide | | | 1H), 3.88 (s, 3H), 3.82 (s, 3H), |
| | | | | 3.19 (m, 2H), 2.99 (m, 2H), 2.02 (m, 2H), |
| | | | | 1.77 (m, 2H). |
| 8 | 2-(2-fluorophenyl)-4- | 370.45 | 371 | 1.77-1.44 (m, 4H), 2.87 (m, 2H), |
| | [(3S)-piperidin-3- | | | 3.12 (m, 2H), 4.19 (m, 1H), 7.46-7.31 (m, |
| | ylamino]thieno[3,2- | | | 5H), 7.81 (m, 2H), 7.94 (br s, 1H), |
| | c]pyridine-7-carboxamide | | | 8.26 (s, 1H), 8.53 (s, 1H) |
| 9 | 4-[(3S)-piperidin-3- | 358.49 | 359 | 9.42 (s, 1H), 8.94 (s, 1H), 8.55 (s, 1H), |
| | ylamino]-2-(2- | | | 8.34 (s, 2H), 7.65 (d, 1H), 7.46 (d, |
| | thienyl)thieno[3,2- | | | 1H), 7.40 (m, 1H), 7.17 (m, 1H), |
| | c]pyridine-7-carboxamide | | | 4.57 (m, 1H), 3.50 (m, 1H), 3.21 (m, 1H), |
| | | | | 2.96 (m, 2H), 2.05 (m, 2H), 1.76 (m, |
| | | | | 2H). |
| 10 | 2-(3,5-difluorophenyl)-4- | 388.44 | 389 | 9.72 (s, 1H), 9.43 (s, 1H), 9.16 (s, 2H), |
| | [(3S)-piperidin-3- | | | 8.61 (s, 1H), 8.51 (s, 1H), 7.74 (s, 1H), |
| | ylamino]thieno[3,2- | | | 7.47 (d, 2H), 7.33 (m, 1H), 4.67 (m, |
| | c]pyridine-7-carboxamide | | | 1H), 3.28 (m, 2H), 3.04 (m, 2H), |
| | | | | 2.06 (m, 2H), 1.83 (m, 2H). |
| 11 | 2-(3,4-dichlorophenyl)-4- | 421.35 | 421 | 9.45 (s, 1H), 9.04 (s, 1H), 8.77 (s, 1H), |
| | [(3S)-piperidin-3- | | | 8.59 (s, 1H), 8.28 (m, 1H), 7.97 (s, |
| | ylamino]thieno[3,2- | | | 1H), 7.77 (m, 2H), 7.59 (m, 2H), |
| | c]pyridine-7-carboxamide | | | 4.60 (m, 1H), 3.24 (m, 2H), 3.01 (m, 2H), |
| | | | | 2.01 (m, 2H), 1.78 (m, 2H). |
| 12 | 4-[(3S)-piperidin-3- | 420.46 | 421 | 9.62 (s, 1H), 9.08 (s, 1H), 8.97 (s, 1H), |
| | ylamino]-2-[3- | | | 8.61 (s, 1H), 8.42 (m, 1H), 8.07 (s, |
| | (trifluoromethyl)phenyl]thieno[3, | | | 2H), 7.79 (s, 2H), 7.69 (s, 1H), |
| | 2-c]pyridine-7- | | | 4.64 (m, 1H), 3.21 (m, 2H), 3.03 (m, 2H), |
| | carboxamide | | | 2.05 (m, 2H), 1.81 (m, 2H). |
| 13 | 4-[(3S)-azepan-3- | 366.49 | 367 | 1.71-1.58 (m, 1H), 1.94-1.83 (m, 4H), |
| | ylamino]-2- | | | 2.17-2.07 (m, 1H), 3.31-3.20 (m, 2H), |
| | phenylthieno[3,2- | | | 3.45-3.31 (m, 2H), 4.71 (m, 1H), |
| | c]pyridine-7-carboxamide | | | 7.31 (m, 2H), 7.43 (m, 2H), 7.51 (m, 2H), |
| | | | | 7.76 (m, 2H), 8.23 (br s, 1H), 8.46 (m, |
| | | | | 1H), 8.54 (s, 1H), 9.10 (br s, 1H), |
| | | | | 9.31 (br s, 1H) |
| 14 | 2-(4-chlorophenyl)-4- | 386.91 | 388 | 9.29 (s, 1H), 8.94 (s, 1H), 8.56 (s, 2H), |
| | [(3S)-piperidin-3- | | | 8.23 (s, 1H), 7.76 (d, 2H), 7.60 (d, |
| | ylamino]thieno[3,2- | | | 3H), 4.57 (m, 1H), 3.45 (m, 2H), |
| | c]pyridine-7-carboxamide | | | 3.01 (m, 2H), 2.03 (m, 2H), 1.77 (m, 2H). |
| 15 | 2-phenyl-4-[(3S)- | 338.43 | 339 | 2.20-2.12 (m, 2H), 3.55-3.22 (m, 3H), |
| | pyrrolidin-3- | | | 4.09 (m, 1H), 4.76 (m, 1H), 7.50-7.32 (m, |
| | ylamino]thieno[3,2- | | | 3H), 7.90-7.75 (m, 3H), 8.23-8.07 (m, |
| | c]pyridine-7-carboxamide | | | 2H), 8.56 (s, 1H), 9.05-8.90 (m, 2H) |
| 16 | 4-[(2-aminoethyl)amino]- | 312.4 | 313 | 3.16 (m, 2H), 3.82 (m, 2H), 7.31 (m, 1H), |
| | 2-phenylthieno[3,2- | | | 7.40 (m, 1H), 7.51 (m, 2H), 8.00 (br s, |
| | c]pyridine-7-carboxamide | | | 2H), 8.20 (br s, 1H), 8.32 (br s, 1H), |
| | | | | 8.53 (s, 1H) |
| 17 | 2-phenyl-4-[(3R)- | 352.46 | 353 | 1.73-1.66 (m, 2H), 2.01-1.95 (m, 2H), |
| | piperidin-3- | | | 3.00-2.95 (2H), 3.15 (m, 1H), 3.24 (m, |
| | ylamino]thieno[3,2- | | | 1H), 4.56 (m, 1H), 7.46 (m, 3H), |
| | c]pyridine-7-carboxamide | | | 7.75-7.73 (m, 3H), 8.02 (br s, 1H), 8.37 (s, |
| | | | | 1H), 8.55 (s, 1H), 8.96 (br s, 1H), |
| | | | | 9.27 (br s, 1H) |
| 18 | 2-(4-tert-butylphenyl)-4- | 408.57 | 409 | 8.51 (s, 1H), 8.16 (s, 1H), 7.93 (br s, 1H), |
| | [(3S)-piperidin-3- | | | 7.67 (d, 2H), 7.51 (d, 2H), 7.28 (br s, 1H), |
| | ylamino]thieno[3,2- | | | 7.16 (d, 2H), 4.17 (m, 1H), 3.13 (d, 1H), |
| | c]pyridine-7-carboxamide | | | 2.83 (d, 1H), 2.44 (m, 2H), 1.99 (m, 1H), |
| | | | | 1.67 (m, 1H), 1.51 (m, 2H), 1.32 (s, 9H) |
| 19 | 2-(4-fluorophenyl)-4- | 370.45 | 371 | 1.77-1.66 (m, 2H), 2.13-1.92 (m, 2H), |
| | [(3S)-piperidin-3- | | | 2.84 (m, 2H), 3.25 (m, 1H), 3.49 (m, |
| | ylamino]thieno[3,2- | | | 1H), 4.49 (m, 1H), 7.35 (m, 2H), |
| | c]pyridine-7-carboxamide | | | 7.54 (br s, 1H), 7.73 (m, 2H), 8.04 (br s, |
| | | | | 1H), 8.11 (s, 1H), 8.54 (s, 1H), |
| | | | | 8.72 (br s, 2H) |
| 20 | 2-(4-acetylphenyl)-4- | 394.5 | 395 | 9.40 (s, 1H), 8.97 (s, 1H), 8.82 (s, 1H), |
| | [(3S)-piperidin-3- | | | 8.59 (s, 1H), 8.29 (s, 1H), 8.04 (d, 2H), |
| | ylamino]thieno[3,2- | | | 7.91 (d, 2H), 7.60 (m, 1H), 4.60 (m, |
| | c]pyridine-7-carboxamide | | | 1H), 3.21 (m, 2H), 3.02 (m, 2H), |
| | | | | 2.62 (s, 3H), 2.03 (m, 2H), 1.79 (m, 2H). |
| 21 | 2-(3-chloro-4- | 404.9 | 406 | 9.73 (s, 1H), 9.45 (s, 1H), 9.13 (s, 1H), |
| | fluorophenyl)-4-[(3S)- | | | 9.03 (s, 1H), 8.60 (s, 1H), 8.52 (s, 1H), |
| | piperidin-3- | | | 7.97 (d, 1H), 7.78 (m, 1H), 7.59 (t, |
| | ylamino]thieno[3,2- | | | 1H), 4.66 (m, 1H), 3.48 (m, 2H), |
| | c]pyridine-7-carboxamide | | | 3.22 (m, 2H), 2.02 (m, 2H), 1.83 (m, 2H). |
| 22 | 4-[(3S)-piperidin-3- | 406.43 | 407 | 9.35 (s, 1H), 8.94 (s, 1H), 8.61 (s, 2H), |
| | ylamino]-2-(3,4,5- | | | 8.26 (s, 1H), 7.70 (m, 1H), 7.52 (m, |
| | trifluorophenyl)thieno[3,2- | | | 2H), 7.39 (d, 2H), 4.58 (m, 1H), |
| | c]pyridine-7- | | | 3.19 (m, 2H), 2.98 (m, 2H), 2.04 (m, 2H), |
| | carboxamide | | | 1.77 (m, 2H). |
| 23 | 2-(4-chloro-3- | 404.9 | 406 | 9.41 (s, 1H), 8.96 (s, 1H), 8.68 (s, 1H), |
| | fluorophenyl)-4-[(3S)- | | | 8.61 (s, 1H), 8.28 (s, 1H), 7.83 (t, 1H), |
| | piperidin-3- | | | 7.66 (t, 1H), 7.58 (s, 1H), 7.39 (t, 1H), |
| | ylamino]thieno[3,2- | | | 4.59 (m, 1H), 3.21 (m, 2H), 3.00 (m, |
| | c]pyridine-7-carboxamide | | | 2H), 2.04 (m, 2H), 1.79 (m, 2H). |
| 24 | 2-{4- | 409.51 | 410 | 9.56 (s, 1H), 9.01 (s, 1H), 8.90 (s, 1H), |
| | [(methylamino)carbonyl]phenyl}- | | | 8.58 (d, 2H), 7.96 (d, 2H), 7.88 (d, |
| | 4-[(3S)- | | | 2H), 7.68 (m, 1H), 4.60 (m, 1H), |
| | piperidin-3- | | | 3.21 (m, 2H), 3.02 (m, 2H), 2.82 (d, 3H), |
| | ylamino]thieno[3,2- | | | 2.04 (m, 2H), 1.80 (m, 2H). |
| | c]pyridine-7-carboxamide | | | |
| 25 | 2-(2,6-difluoropyridin-3- | 389.43 | 390 | 9.32 (s, 1H), 8.93 (s, 1H), 8.61 (s, 1H), |
| | yl)-4-[(3S)-piperidin-3- | | | 8.54 (m, 1H), 8.24 (s, 1H), 7.71 (d, |
| | ylamino]thieno[3,2- | | | 1H), 7.54 (m, 1H), 7.40 (d, 1H), |
| | c]pyridine-7-carboxamide | | | 4.58 (m, 1H), 3.18 (m, 2H), 3.03 (m, 2H), |
| | | | | 2.03 (m, 2H), 1.76 (m, 2H). |
| 26 | 4-[(3S)-piperidin-3- | 463.6 | 464 | 9.46 (s, 1H), 8.97 (s, 1H), 8.80 (s, 1H), |
| | ylamino]-2-[4-(piperidin- | | | 8.57 (s, 1H), 8.31 (s, 1H), 7.83 (d, 2H), |
| | 1- | | | 7.68 (m, 1H), 7.51 (d, 2H), 4.59 (m, |
| | ylcarbonyl)phenyl]thieno[3, | | | 1H), 3.33 (m, 4H), 3.21 (m, 2H), |
| | 2-c]pyridine-7- | | | 3.03 (m, 2H), 2.05 (m, 2H), 1.79 (m, 2H), |
| | carboxamide | | | 1.58 (m, 6H). |
| 27 | 2-(3-{[(methylsulfonyl)amino]methyl}phenyl)- | 459.59 | 460 | 9.46 (s, 1H), 8.96 (s, 1H), 8.67 (s, 1H), |
| | 4- | | | 8.56 (s, 1H), 8.32 (s, 1H), 7.71 (m, 4H), |
| | [(3S)-piperidin-3- | | | 7.51 (t, 1H), 7.41 (d, 1H), 4.59 (m, 1H), |
| | ylamino] thieno[3,2- | | | 4.24 (d, 2H), 3.22 (m, 2H), 3.03 (m, 2H), |
| | c]pyridine-7-carboxamide | | | 2.92 (s, 3H), 2.08 (m, 2H), 1.79 (m, 2H). |
| 28 | 2-[4- | 458.6 | 459 | 9.41 (s, 1H), 9.00 (s, 1H), 8.89 (s, 1H), |
| | (isopropylsulfonyl)phenyl]- | | | 8.61 (s, 1H), 8.30 (m, 1H), 8.01 (q, |
| | 4-[(3S)-piperidin-3- | | | 3H), 7.71 (d, 1H), 7.62 (s, 1H), 4.60 (s, |
| | ylamino]thieno[3,2- | | | 1H), 3.35 (m, 2H), 3.00 (m, 2H), |
| | c]pyridine-7-carboxamide | | | 2.04 (m, 2H), 1.79 (m, 2H), 1.65 (m, 1H), |
| | | | | 1.21 (s, 3H), 1.19 (s, 3H). |
| 29 | 2-(3-chlorophenyl)-4- | 386.91 | 387 | 9.56 (s, 1H), 9.01 (s, 1H), 8.88 (s, 1H), |
| | [(3S)-piperidin-3- | | | 8.58 (s, 1H), 8.37 (s, 1H), 7.82 (s, 1H), |
| | ylamino]thieno[3,2- | | | 7.73 (d, 1H), 7.62 (brs, 1H), 7.55 (t, |
| | c]pyridine-7-carboxamide | | | 1H), 7.50 (d, 1H), 4.63 (m, 1H), |
| | | | | 3.48 (d, 2H), 3.17 (m, 2H), 2.06 (m, 2H), |
| | | | | 1.81 (m, 2H). |
| 30 | 2-(3,4-difluorophenyl)-4- | 388.44 | 389 | 9.36 (s, 1H), 8.95 (s, 1H), 8.65 (s, 1H), |
| | [(3S)-piperidin-3- | | | 8.57 (s, 1H), 8.23 (s, 1H), 7.79 (t, 1H), |
| | ylamino]thieno[3,2- | | | 7.59 (m, 3H), 4.58 (m, 1H), 3.17 (d, 2H), |
| | c]pyridine-7-carboxamide | | | 3.02 (m, 2H), 2.00 (m, 2H), 1.77 (m, 2H). |
| 31 | 2-(5-acetyl-2-thienyl)-4- | 400.53 | 401 | 9.25 (s, 1H), 8.94 (s, 1H), 8.60 (s, 1H), |
| | [(3S)-piperidin-3- | | | 8.47 (s, 1H), 8.19 (s, 1H), 7.96 (d, 1H), |
| | ylamino]thieno[3,2- | | | 7.54 (d, 2H), 4.56 (s, 1H), 3.17 (d, |
| | c]pyridine-7-carboxamide | | | 2H), 2.99 (m, 2H), 2.56 (s, 3H), |
| | | | | 2.02 (m, 2H), 1.76 (m, 2H). |
| 32 | 2-{3- | 423.54 | 424 | 9.53 (s, 1H), 9.00 (s, 1H), 8.81 (s, 1H), |
| | [(dimethylamino)carbonyl]phenyl}- | | | 8.56 (s, 1H), 8.33 (s, 1H), 7.83 (d, 1H), |
| | 4-[(3S)- | | | 7.77 (s, 1H), 7.58 (t, 2H), 7.44 (d, 1H), |
| | piperidin-3- | | | 4.60 (s, 1H), 3.52 (d, 2H), 3.17 (m, |
| | ylamino]thieno[3,2- | | | 2H), 3.00 (d, 6H), 2.05 (m, 2H), |
| | c]pyridine-7-carboxamide | | | 1.79 (m, 2H). |
| 33 | 2-(2-fluoropyridin-4-yl)- | 371.44 | 372 | 9.50 (s, 1H), 9.15 (s, 1H), 9.06 (s, 1H), |
| | 4-[(3S)-piperidin-3- | | | 8.63 (s, 1H), 8.35 (d, 1H), 7.77 (d, |
| | ylamino]thieno[3,2- | | | 1H), 7.63 (m, 1H), 7.47 (s, 1H), |
| | c]pyridine-7-carboxamide | | | 4.63 (s, 1H), 3.49 (d, 2H), 3.16 (m, 2H), |
| | | | | 2.02 (m, 2H), 1.80 (m, 2H). |
| 34 | 2-[2-(4-methylpiperazin- | 451.6 | 452 | 11.20 (s, 1H), 9.82 (s, 1H), 9.62 (s, |
| | 1-yl)pyridin-4-yl]-4- | | | 1H), 9.14 (s, 1H), 9.01 (s, 1H), 8.55 (d, |
| | [(3S)-piperidin-3- | | | 3H), 7.99 (d, 1H), 7.73 (m, 1H), |
| | ylamino]thieno[3,2- | | | 7.12 (d, 1H), 4.68 (s, 1H), 4.50 (d, 2H), |
| | c]pyridine-7-carboxamide | | | 3.49 (d, 4H), 3.36 (d, 2H), 3.11 (m, |
| | | | | 4H), 2.79 (d, 3H), 2.05 (m, 2H), |
| | | | | 1.86 (m, 2H). |
| 35 | 2-[3- | 409.51 | 410 | 10.18 (s, 1H), 9.42 (s, 1H), 8.95 (d, |
| | (acetylamino)phenyl]-4- | | | 1H), 8.60 (s, 1H), 8.54 (s, 1H), |
| | [(3S)-piperidin-3- | | | 8.34 (brs, 1H), 7.69 (brs, 1H), 7.46 (m, 3H), |
| | ylamino]thieno[3,2- | | | 4.58 (s, 1H), 3.49 (d, 2H), 3.00 (m, |
| | c]pyridine-7-carboxamide | | | 2H), 2.08 (s, 3H), 2.05 (m, 2H), |
| | | | | 1.77 (m, 2H). |
| 36 | 4-[(3S)-piperidin-3- | 403.51 | 404 | 9.54 (s, 1H), 9.41 (s, 1H), 9.10 (s, 1H), |
| | ylamino]-2-quinolin-3- | | | 9.02 (s, 1H), 8.85 (s, 1H), 8.61 (s, 1H), |
| | ylthieno[3,2-c]pyridine-7- | | | 8.37 (brs, 1H), 8.16 (m, 2H), 7.86 (t, |
| | carboxamide | | | 1H), 7.73 (t, 1H), 7.70 (brs, 1H), |
| | | | | 4.65 (s, 1H), 3.49 (d, 2H), 3.19 (m, 2H), |
| | | | | 2.08 (m, 2H), 1.82 (m, 2H). |
| 37 | 2-(6-fluoro-2- | 385.47 | 386 | 9.37 (s, 1H), 8.93 (s, 1H), 8.59 (s, 1H), |
| | methylpyridin-3-yl)-4- | | | 8.28 (s, 1H), 8.18 (brs, 1H), 8.06 (t, |
| | [(3S)-piperidin-3- | | | 1H), 7.52 (brs, 1H), 7.17 (m, 1H), |
| | ylamino]thieno[3,2- | | | 4.59 (s, 1H), 3.17 (d, 2H), 2.99 (m, 2H), |
| | c]pyridine-7-carboxamide | | | 2.62 (s, 3H), 2.05 (m, 2H), 1.75 (m, |
| | | | | 2H). |
| 38 | 2-(5-fluoro-6-oxo-1,6- | 387.44 | 388 | 12.54 (s, 1H), 9.39 (s, 1H), 8.94 (s, |
| | dihydropyridin-3-yl)-4- | | | 1H), 8.54 (s, 1H), 8.48 (s, 1H), |
| | [(3S)-piperidin-3- | | | 8.18 (brs, 1H), 7.76 (d, 2H), 7.66 (s, 1H), |
| | ylamino]thieno[3,2- | | | 7.55 (s, 1H), 4.57 (s, 1H), 3.17 (d, 2H), |
| | c]pyridine-7-carboxamide | | | 3.03 (m, 2H), 2.05 (m, 2H), 1.77 (m, |
| | | | | 2H). |
| 39 | 2-(5-chloro-6-oxo-1,6- | 403.89 | 404 | 12.54 (s, 1H), 9.08 (s, 1H), 8.87 (s, |
| | dihydropyridin-3-yl)-4- | | | 1H), 8.54 (s, 1H), 8.18 (s, 1H), 8.09 (s, |
| | [(3S)-piperidin-3- | | | 1H), 7.70 (m, 3H), 7.45 (s, 1H), |
| | ylamino]thieno[3,2- | | | 4.51 (s, 1H), 3.59 (d, 2H), 2.95 (m, 2H), |
| | c]pyridine-7-carboxamide | | | 2.01 (m, 2H), 1.72 (m, 2H). |
| 40 | 2-(6-morpholin-4- | 438.55 | 439 | 9.56 (s, 1H), 8.99 (s, 1H), 8.72 (s, 1H), |
| | ylpyridin-3-yl)-4-[(3S)- | | | 8.51 (s, 2H), 8.37 (s, 1H), 8.00 (d, 1H), |
| | piperidin-3- | | | 7.67 (m, 1H), 7.10 (d, 1H), 4.60 (s, |
| | ylamino]thieno[3,2- | | | 1H), 3.73 (m, 2H), 3.59 (m, 4H), |
| | c]pyridine-7-carboxamide | | | 3.49 (m, 2H), 3.17 (m, 2H), 3.04 (m, 2H), |
| | | | | 2.04 (m, 2H), 1.80 (m, 2H). |
| 41 | 2-[4- | 395.53 | 396 | 9.26 (br s, 1H), 8.82 (br s, 1H), 8.44 (s, |
| | (dimethylamino)phenyl]- | | | 1H), 8.29 (m, 2H), 7.60 (m, 3H), |
| | 4-[(3S)-piperidin-3- | | | 6.85 (d, 2H), 4.50 (m, 1H), 3.18 (m, 2H), |
| | ylamino]thieno[3,2- | | | 3.11 (m, 2H), 2.97 (s, 6H), 2.02 (m, |
| | c]pyridine-7-carboxamide | | | 2H), 1.75 (m, 2H) |
| 42 | 2-[3- | 395.53 | 396 | 9.40 (br s, 1H), 8.92 (br s, 1H), |
| | (dimethylamino)phenyl]- | | | 8.61 (m, 1H), 8.51 (s, 1H), 8.28 (m, 1H), |
| | 4-[(3S)-piperidin-3- | | | 7.60 (m, 1H), 7.33 (m, 2H), 7.16 (m, |
| | ylamino]thieno[3,2- | | | 1H), 6.87 (m, 1H), 4.56 (m, 1H), |
| | c]pyridine-7-carboxamide | | | 3.47 (m, 2H), 3.15 (m, 2H), 3.00 (s, 6H), |
| | | | | 2.00 (m, 2H), 1.78 (m, 2H) |
| 43 | 4-[methyl(piperidin-3- | 366.49 | 367 | 9.26 (br s, 1H), 8.92 (br s, 1H), 8.59 (s, |
| | yl)amino]-2- | | | 1H), 8.18 (br s, 1H), 7.94 (s, 1H), |
| | phenylthieno[3,2- | | | 7.85 (m, 2H), 7.46 (m, 2H), 7.40 (m, 1H), |
| | c]pyridine-7-carboxamide | | | 4.83 (m, 1H), 3.42 (m, 1H), 3.29 (s, |
| | | | | 3H), 3.22 (m, 2H), 2.89 (m, 1H), |
| | | | | 2.00-1.79 (m, 4H) |
| 44 | 2-[3- | 381.5 | 382 | 9.20 (br s, 1H), 8.82 (br s, 1H), 8.49 (s, |
| | (aminomethyl)phenyl]-4- | | | 1H), 8.46 (m, 1H), 8.24 (m, 3H), |
| | [(3S)-piperidin-3- | | | 8.06 (br s, 1H), 7.80 (s, 1H), 7.74 (d, 1H), |
| | ylamino]thieno[3,2- | | | 7.49 (t, 1H), 7.40 (m, 2H), 4.48 (m, |
| | c]pyridine-7-carboxamide | | | 1H), 4.03 (dd, 2H), 3.13 (m, 2H), |
| | | | | 2.91 (m, 2H), 1.94 (m, 2H), 1.68 (m, 2H) |
| 45 | 2-pyridin-3-yl-4-[(3S)- | 339.42 | 340 | 9.50 (br s, 3H), 9.19 (s, 1H), 9.10 (s, |
| | pyrrolidin-3- | | | 1H), 8.73 (d, 1H), 8.62 (s, 1H), |
| | ylamino]thieno[3,2- | | | 8.45 (d, 2H), 7.82 (dd, 1H), 7.73 (br s, 1H), |
| | c]pyridine-7-carboxamide | | | 4.99 (m, 1H), 3.50 (m, 2H), 3.33 (m, |
| | | | | 2H), 2.35 (m, 1H), 2.26 (m, 1H) |
| 46 | 2-pyridin-3-yl-4-[(3R)- | 339.42 | 340 | 9.70 (br s, 2H), 9.54 (br s, 1H), 9.19 (s, |
| | pyrrolidin-3- | | | 1H), 9.09 (s, 1H), 8.73 (d, 1H), 8.66 (s, |
| | ylamino]thieno[3,2- | | | 1H), 8.43 (d, 2H), 7.80 (dd, 1H), 7.73 (br |
| | c]pyridine-7-carboxamide | | | s, 1H), 5.05 (m, 1H), 3.50 (m, 2H), |
| | | | | 3.33 (m, 2H), 2.35 (m, 1H), 2.26 (m, 1H) |
| 47 | 2-(4-methyl-3,4-dihydro- | 423.54 | 424 | 9.70 (m, 1H), 8.95 (m, 1H), 8.65 (m, |
| | 2H-1,4-benzoxazin-7-yl)- | | | 1H), 8.44 (m, 2H), 7.73 (m, 1H), |
| | 4-[(3S)-piperidin-3- | | | 7.22 (d, 1H), 7.08 (s, 1H), 6.78 (d, 1H), |
| | ylamino]thieno[3,2- | | | 4.58 (m, 1H), 4.26 (m, 2H), 3.47 (m, |
| | c]pyridine-7-carboxamide | | | 2H), 3.23 (m, 2H), 3.03 (m, 2H), |
| | | | | 2.90 (s, 3H), 2.00 (m, 2H), 1.80 (m, 2H) |
| 48 | 2-(3′-bromobiphenyl-3- | 507.45 | 508 | 8.90 (m, 2H), 8.56 (s, 1H), 8.36 (m, |
| | yl)-4-[(3S)-piperidin-3- | | | 1H), 7.97 (d, 2H), 7.76 (m, 2H), |
| | ylamino]thieno[3,2- | | | 7.68 (m, 1H), 7.60 (m, 3H), 7.47 (m, 2H), |
| | c]pyridine-7-carboxamide | | | 4.55 (m, 1H), 3.24 (m, 2H), 2.91 (m, |
| | | | | 2H), 2.00 (m, 2H), 1.74 (m, 2H) |
| 49 | 2-(1-benzyl-1H-pyrazol- | 432.55 | 433 | 9.45 (br s, 1H), 8.90 (m, 1H), 8.48 (s, 1H), |
| | 4-yl)-4-[(3S)-piperidin-3- | | | 8.41-8.14 (m, 2H), 8.33 (s, 1H), 7.85 (s, |
| | ylamino]thieno[3,2- | | | 1H), 7.61 (s, 1H), 7.39-7.27 (m, 5H), |
| | c]pyridine-7-carboxamide | | | 5.38 (s, 2H), 4.54 (m, 1H), 3.47 (m, 1H), |
| | | | | 3.18 (m, 1H), 3.08 (m, 1H), 2.97 (m, 1H), |
| | | | | 2.05 (m, 1H), 1.97 (m, 1H), 1.77 (m, 2H) |
| 50 | 2-(1-methyl-1H-pyrazol- | 356.45 | 357 | 9.44 (br s, 1H), 8.89 (m, 1H), 8.47 (s, |
| | 4-yl)-4-[(3S)-piperidin-3- | | | 1H), 8.45-8.20 (m, 2H), 8.18 (s, 1H), |
| | ylamino]thieno[3,2- | | | 7.80 (s, 1H), 7.63 (br s, 1H), 4.54 (m, |
| | c]pyridine-7-carboxamide | | | 1H), 3.89 (s, 3H), 3.47 (m, 1H), |
| | | | | 3.20 (m, 1H), 3.10 (m, 1H), 2.99 (m, 1H), |
| | | | | 2.05 (m, 1H), 1.97 (m, 1H), 1.78 (m, |
| | | | | 2H) |
| 51 | 2-[2-(benzyloxy)phenyl]- | 458.58 | 459 | 8.55 (s, 1H), 8.33 (s, 1H), 7.77 (br s, |
| | 4-[(3S)-piperidin-3- | | | 2H), 7.78 (d, 1H), 7.54 (d, 2H), 7.36 (t, |
| | ylamino]thieno[3,2- | | | 2H), 7.30 (t, 2H), 7.23 (d, 1H), |
| | c]pyridine-7-carboxamide | | | 7.19 (br s, 1H), 7.08 (t, 1H), 5.36 (s, 2H), |
| | | | | 4.61 (br s, 1H), 3.45 (dd, 1H), 3.20 (s, |
| | | | | 3H), 3.15-3.20 (m, 1H), |
| | | | | 2.02-2.09 (m, 2H), 1.76-1.85 (m, 2H) |

### Example 52

### 2-[1-(1,3-benzothiazol-2-ylmethyl)-1H-pyrazol-4-yl]-4-[(3S)-piperidin-3-ylamino]thieno[3,2-c]pyridine-7-carboxamide

Prepared in a similar fashion to Example 1 but using 2-{[4-(4,4,5,5-tetramethyl-1,3,2-dioxaborolan-2-yl)-1H-pyrazol-1-yl]methyl}-1,3-benzothiazole (synthesis described below) as the starting material in step 8. ¹H NMR δ 9.11 (br, 1H), 8.81 (br, 1H), 8.50 (s, 1H), 8.44 (s, 1H), 8.10 (br, 2H), 8.08 (d, 1H), 8.01 (d, 1H), 7.94 (s, 1H), 7.55-7.42 (m, 3H), 5.92 (s, 2H), 4.51 (br, 1H), 3.46 (m, 1H), 3.20 (m, 1H), 2.95 (m, 2H), 2.09-1.92 (m, 2H), 1.82-1.65 (m, 2H). LCMS (ES, M+H=490).

2-{[4-(4,4,5,5-tetramethyl-1,3,2-dioxaborolan-2-yl)-1H-pyrazol-1-yl]methyl}-1,3-benzothiazole. To a solution of 4-(4,4,5,5-tetramethyl-1,3,2-dioxaborolan-2-yl)-1H-pyrazole (100 mg, 0.515 mmol) and 2-(bromomethyl)-1,3-benzothiazole (118 mg, 0.515 mmol) in DMF (1.7 mL) is added NaH (60% dispersion in oil, 23 mg, 0.567 mmol) at 23° C. The reaction mixture is stirred overnight. The reaction is quenched by addition of NH₄Cl and the product is extracted into EtOAc. The organic layers are washed with brine, dried over Na₂SO₄ and concentrated in vacuo.

Examples 53-57 are made in a similar fashion as example 1 using appropriate starting materials.

| | | | MS | |
|---|---|---|---|---|
| | | Mw | (ES, | ¹H NMR (300 MHz; d₆-DMSO; δ |
| Example | IUPAC Name | (g/mol) | M + H) | ppm) unless otherwise noted |
| 53 | 4-[(3S)-piperidin-3- | 433.54 | 434 | 9.63 (br, 1H), 8.96 (br, 1H), 8.63 (d, |
| | ylamino]-2-[1-(pyridin- | | | 1H), 8.50 (s, 1H), 8.47 (br, 2H), |
| | 2-ylmethyl)-1H- | | | 8.40 (s, 1H), 7.96 (m, 1H), 7.90 (s, 1H), |
| | pyrazol-4-yl]thieno[3,2- | | | 7.69 (br, 1H), 7.48 (dd, 1H), 7.32 (d, |
| | c]pyridine-7- | | | 1H), 5.58 (s, 2H), 4.59 (m, 1H), |
| | carboxamide | | | 3.47 (m, 1H), 3.18 (m, 2H), 2.99 (m, 1H), |
| | | | | 2.12-1.91 (m, 2H), 1.85-1.72 (m, 2H) |
| 54 | 4-[(3S)-piperidin-3- | 433.54 | 434 | 9.31 (br, 1H), 8.87 (br, 1H), 8.77 (s, |
| | ylamino]-2-[1-(pyridin- | | | 1H), 8.72 (d, 1H), 8.49 (s, 1H), 8.40 (s, |
| | 3-ylmethyl)-1H- | | | 1H), 8.32-8.08 (m, 3H), 7.88 (s, 1H), |
| | pyrazol-4-yl]thieno[3,2- | | | 7.75 (m, 1H), 7.53 (br, 1H), 5.55 (s, |
| | c]pyridine-7- | | | 2H), 4.53 (m, 1H), 3.46 (m, 1H), |
| | carboxamide | | | 3.19 (m, 1H), 2.99 (m, 2H), 2.10-1.90 (m, |
| | | | | 2H), 1.83-1.67 (m, 2H) |
| 55 | 2-[1-(2-aminoethyl)- | 385.49 | 386 | 9.21 (br, 1H), 8.87 (br, 1H), 8.50 (s, |
| | 1H-pyrazol-4-yl]-4- | | | 1H), 8.23 (s, 1H), 8.04 (br, 4H), |
| | [(3S)-piperidin-3- | | | 7.88 (s, 1H), 7.49-7.21 (m, 1H), 4.54 (m, |
| | ylamino]thieno[3,2- | | | 1H), 4.42 (t, 2H), 3.31 (m, 2H), |
| | c]pyridine-7- | | | 3.19 (m, 2H), 2.95 (m, 2H), 2.05-1.91 (m, |
| | carboxainide | | | 2H), 1.81-1.67 (m, 2H) |
| 56 | 4-[(3S)-piperidin-3- | 433.54 | 434 | 9.36 (br, 1H), 8.92 (br, 1H), 8.79 (d, |
| | ylamino]-2-[1-(pyridin- | | | 2H), 8.51 (s, 1H), 8.42 (s, 1H), |
| | 4-ylmethyl)-1H- | | | 8.24 (br, 2H), 7.95 (s, 1H), 7.60 (d, 2H), |
| | pyrazol-4-yl]thieno[3,2- | | | 7.52 (br, 1H), 5.70 (s, 2H), 4.55 (m, |
| | c]pyridine-7- | | | 1H), 3.46 (m, 1H), 3.18 (m, 1H), |
| | carboxamide | | | 3.00 (m, 2H), 2.08-1.90 (m, 2H), |
| | | | | 1.82-1.67 (m, 2H) |
| 57 | 4-[(3S)-piperidin-3- | 439.57 | 440 | 9.54 (br, 1H), 9.10 (s, 1H), 8.92 (br, |
| | ylamino]-2-[1-(1,3- | | | 1H), 8.48 (s, 1H), 8.38 (br, 1H), |
| | thiazol-4-ylmethyl)-1H- | | | 8.30 (s, 1H), 7.85 (s, 1H), 7.68 (br, 1H), |
| | pyrazol-4-yl]thieno[3,2- | | | 7.65 (s, 1H), 5.53 (s, 1H), 4.56 (m, |
| | c]pyridine-7- | | | 1H), 3.47 (m, 1H), 3.17 (m, 2H), |
| | carboxamide | | | 2.99 (m, 1H), 2.12-1.92 (m, 2H), |
| | | | | 1.84-1.70 (m, 2H) |

### Example 58

### 4-{methyl[(3S)-piperidin-3-yl]amino}-2-phenylthieno[3,2-c]pyridine-7-carboxamide

Prepared in a similar fashion to Example 1 but using tert-butyl (3S)-3-(methylamino)piperidine-1-carboxylate (synthesis described below) as the starting material in step 7. ¹H NMR δ 9.26 (br s, 1H), 8.92 (br s, 1H), 8.59 (s, 1H), 8.18 (br s, 1H), 7.94 (s, 1H), 7.85 (m, 2H), 7.46 (m, 2H), 7.40 (m, 1H), 4.83 (m, 1H), 3.42 (m, 1H), 3.29 (s, 3H), 3.22 (m, 2H), 2.89 (m, 1H), 2.00-1.79 (m, 4H). LCMS (ES, M+H=367).

tert-butyl (3S)-3-(methylamino)piperidine-1-carboxylate. To a solution of formaldehyde (37%, aq.; 0.37 ml, 4.7 mmol) in 20 ml dry MeOH containing 3 Å molecular sieves is added tert-butyl (3S)-3-aminopiperidine-1-carboxylate (1.0 g, 5 mmol). The reaction is stirred under N₂ at rt for ˜30 h, and then NaBH₄ (304 mg, 8 mmol) is added as a solid. The reaction is stirred at rt overnight and then quenched with 1N NaOH (˜10 ml). The phases are separated and the remaining aqueous layer is extracted with ether (3×). The combined organic layers are washed with water and brine, dried, and evaporated to yield a colorless oil (1.04 g, 100%). LCMS (ES, M+H=215).

Examples 59-63 are made in a similar fashion as example 58 using appropriate starting materials.

| | | | MS | |
|---|---|---|---|---|
| | | Mw | (ES, | ¹H NMR (300 MHz; d₆-DMSO; δ |
| Example | IUPAC Name | (g/mol) | M + H) | ppm) unless otherwise noted |
| 59 | 2-(3-fluorophenyl)-4- | 384.48 | 385 | D₂O added: 1.76 (m, 1H), 1.94 (m, H), |
| | {methyl[(3S)-piperidin-3- | | | 2.84 (m, 1H), 3.17 (m, 2H), 3.24 (s, 3H), |
| | yl]amino}thieno[3,2- | | | 3.40 (m, 1H), 4.75 (m, 1H), 7.19 (m, |
| | c]pyridine-7- | | | 1H), 7.52 (m, 1H), 7.60 (d, 1H), 7.67 (d, |
| | carboxamide | | | 1H), 7.92 (s, 1H), 8.52 (s, 1H) |
| 60 | 2-(4-fluorophenyl)-4- | 384.48 | 385 | D₂O added: 1.75 (m, 1H), 1.96 (m, 3H), |
| | {methyl[(3S)-piperidin-3- | | | 2.86 (m, 1H), 3.21 (m, 2H), 3.26 (s, 3H), |
| | yl]amino}thieno[3,2- | | | 3.42 (m, 1H), 4.75 (m, 1H), 7.30 (m, |
| | c]pyridine-7- | | | 2H), 7.85 (m, 3H), 8.55 (s, 1H) |
| | carboxamide | | | |
| 61 | 4-{methyl[(3S)-piperidin- | 372.52 | 373 | D₂O added: 1.76 (m, 1H), 1.95 (m, 3H), |
| | 3-yl]amino}-2-(3-thienyl)thieno[3,2- | | | 2.84 (m, 1H), 3.18 (m, 2H), 3.23 (s, 3H), |
| | c]pyridine-7- | | | 3.401 (m, 1H), 4.69 (m, 1H), 7.61 (m, 2H), |
| | carboxamide | | | 7.75 (s, 1H), 7.92 (s, 1H), 8.50 (s, 1H) |
| 62 | 4-{(2- | 396.51 | 397 | D₂O added: 8.57 (s, 1H), 7.80 (s, 1H), |
| | hydroxyethyl)[(3S)- | | | 7.77 (m, 2H), 7.49 (m, 2H), 7.39 (m, |
| | piperidin-3-yl]amino}-2- | | | 1H), 4.59 (m, 1H), 3.77 (m, 1H), |
| | phenylthieno[3,2- | | | 3.69 (m, 1H), 3.54 (m, 2H), 3.39 (m, 1H), |
| | c]pyridine-7- | | | 3.20 (m, 2H), 2.82 (m, 1H), 1.89 (m, |
| | carboxamide | | | 3H), 1.70 (m, 1H). |
| 63 | 4-{(2- | 402.54 | 403 | D₂O added: 8.56 (s, 1H), 7.96 (s, 1H), |
| | hydroxyethyl)[(3S)- | | | 7.68 (m, 2H), 7.61 (m, 1H), 4.67 (m, |
| | piperidin-3-yl]amino}-2- | | | 1H), 3.88 (m, 1H), 3.73 (m, 1H), |
| | (3-thienyl)thieno[3,2- | | | 3.54 (m, 3H), 3.21 (m, 2H), 2.83 (m, 1H), |
| | c]pyridine-7- | | | 1.90 (m, 3H), 1.74 (m, 1H). |
| | carboxamide | | | |

### Example 64

### 2-[2-(benzyloxy)phenyl]-4-{methyl[(3S)-piperidin-3-yl]amino}thieno[3,2-c]pyridine-7-carboxamide

tert-butyl (3S)-3-[(2-bromo-7-cyanothieno[3,2-c]pyridin-4-yl)(methyl)amino]piperidine-1-carboxylate. tert-Butyl (3S)-3-[(2-bromo-7-cyanothieno[3,2-c]pyridin-4-yl)amino]piperidine-1-carboxylate (240 mg) is dissolved in NMP (10 mL) under nitrogen. Sodium hydride (63 mg) is added and the reaction is left for 20 min. Methyl iodide (108 μL) is added drop wise and the reaction is allowed to stir at rt for 1 hour. Once the reaction is complete (LCMS 452.69 M+H), the reaction mixture is poured into water (120 mL) and extracted with EtOAc. The organics are washed with water and brine and dried over MgSO₄ before reducing in vacuo. The residue is purified on silica column eluting with 30-50% EtOAc/iso-Hexane. The product fractions are combined and reduced in vacuo to give a yellow gum (245 mg, 91%).

tert-butyl (3S)-3-[[7-(aminocarbonyl)-2-bromothieno[3,2-c]pyridin-4-yl](methyl)amino]piperidine-1-carboxylate. tert-Butyl (3S)-3-[(2-bromo-7-cyanothieno[3,2-c]pyridin-4-yl)(methyl)amino]piperidine-1-carboxylate (245 mg) is dissolved in t-butanol (10 mL) and powdered KOH (110 mg) is added. The resulting reaction mixture is heated to 80° C. for 30 min and LCMS confirms complete reaction (468.93 M+H). The reaction mixture is reduced in vacuo and azeotroped with MeOH. The residue is dissolved in CH₂Cl₂ and washed with water and brine and dried over MgSO₄ and diluted with MeOH before applying to an ion exchange column and eluting with MeOH followed by MeOH/NH₃. The product eluted in the basic fractions and is reduced in vacuo to a yellow solid (207 mg).

tert-butyl (3S)-3-[{7-(aminocarbonyl)-2-[2-(benzyloxy)phenyl]thieno[3,2-c]pyridin-4-yl}(methyl)amino]piperidine-1-carboxylate. To tert-butyl (3S)-3-[[7-(aminocarbonyl)-2-bromothieno[3,2-c]pyridin-4-yl] (methyl)amino]piperidine-1-carboxylate (207 mg) is added 2-Benzyloxyphenylboronic acid (151 mg), Pd(PPh₃)₄ (51 mg) and cesium carbonate (432 mg). The mixture is slurried in 10 mL of dioxane:H₂O (4:1) and heated to 80° C. for 1 h. LCMS indicated completion of reaction (573.09 M+H). The reaction mixture is concentrated in vacuo and redissolved in CH₂C12, washed with water, brine and dried over MgSO₄. The residue is purified on 40 g silica column eluting with 30-100% EtOAc/Hexane. The product fractions are combined and reduced in vacuo to give the product as a colorless gum (182 mg).

2-[2-(benzyloxy)phenyl]-4-{methyl[(3S)-piperidin-3-yl]amino}thieno[3,2-c]pyridine-7-carboxamide. tert-butyl (3S)-3-[{7-(aminocarbonyl)-2-[2-(benzyloxy)phenyl]thieno[3,2-c]pyridin-4-yl}(methyl)amino]piperidine-1-carboxylate (182 mg) is dissolved in MeOH 6 ml and 1.5 mL of 4.0M HCl in Dioxane is added. The reaction mixture is then stirred at room temp for 3 hr. LCMS indicates when the reaction is complete (472.87 M+H). After the reaction is complete the reaction mixture is reduced in vacuo carefully 30° C. bath temp and the residue is partitioned between CH₂Cl₂ and a few drops MeOH/NH₃ and saturated NaHCO₃. The organics are washed with brine and dried over MgSO₄ and dry loaded onto silica before purifying on a 12 g silica column eluting with 1-12% MeOH/NH₃/CH₂Cl₂. The cleanest product fractions are combined and reduced in vacuo to give a white solid which is triturated with ether, filtered and dried to give the title product 85 mg. ¹H NMR (500 MHz, DMSO-d₆) δ 1.40-1.55 (m, 1H), 1.62-1.75 (m, 2H), 1.75-1.84 (m, 1H), 2.32-2.43 (m, 1H), 2.60-2.70 (m, 1H), 2.79-2.87 (m, 1H), 2.87-2.97 (m, 1H), 2.91 (s, 3H), 4.36-4.46 (m, 1H), 5.28 (s, 2H), 7.09 (t, 1H), 7.25-7.32 (m, 1H), 7.32-7.46 (m, 5H), 7.55 (d, 2H), 7.73 (d, 1H), 7.82-8.04 (m, 1H), 7.99 (s, 1H), 8.55 (s, 1H). LCMS (ES, M+H=473).

### Example 65

### 2-phenyl-4-{(2-phenylethyl)[(3S)-piperidin-3-yl]amino}thieno[3,2-c]pyridine-7-carboxamide

Prepared in a similar fashion to Example 1 but using tert-butyl (3S)-3-[(2-phenylethyl)amino]piperidine-1-carboxylate (synthesis described below) as the starting material in step 7. ¹H NMR (D₂O added) δ 8.63 (s, 1H), 767 (m, 2H), 7.53 (s, 1H), 7.44 (m, 3H), 7.14-7.23 (m, 5H), 4.50 (m, 1H), 3.85 (m, 2H), 3.15 (m, 3H), 2.80 (m, 3H), 1.80-1.87 (m, 3H), 1.66 (m, 1H). LCMS (ES, M+H=457).

tert-butyl (3S)-3-[(2-phenylethyl)amino]piperidine-1-carboxylate. tert-butyl (3S)-3-aminopiperidine-1-carboxylate (1 g, 5 mmol), (2-bromoethyl)benzene (925 mg, 0.69 ml, 5 mmol) and potassium carbonate (1.73 g, 12.5 mmol) are added to a microwave tube and DMF (6 mL) is added. The mixture is heated at 90° C. for 1 h. The phases are partitioned between EtOAc and water. The organic layer is washed with water and brine, dried and evaporated. The mixture is purified by MPLC (EtOAC/Hexane) to give 794 mg (52%) of the title compound as a colorless oil LCMS (ES, M+H=305).

Examples 66-68 are made in a similar fashion to example 65 using the appropriate starting materials.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 66 | 4-{(4- | 430.59 | 431 | D₂O added: 8.53 (s, 1H), 7.92 (s, 1H), |
| | hydroxybutyl)[(3S)- | | | 7.65 (m, 1H), 7.57 (m, 2H), 4.53 (m, |
| | piperidin-3-yl]amino}- | | | 1H), 3.60 (m, 2H), 3.35 (m, 2H), |
| | 2-(3-thienyl)thieno[3,2- | | | 3.21 (m, 3H), 3.21 (m, 1H), 2.81 (m, 1H), |
| | c]pyridine-7- | | | 1.96 (m, 3H), 1.74 (m, 1H), 1.45 (m, 3H). |
| | carboxamide | | | |
| 67 | 2-(3-fluorophenyl)-4-{(2- | 474.6 | 475 | D₂O added: 8.71 (s, 1H), 7.68 (s, 1H), |
| | phenylethyl)[(3S)- | | | 7.58 (m, 3H), 7.28 (m, 4H), 7.21 (m, 2H), |
| | piperidin-3- | | | 4.59 (m, 1H), 3.80 (m, 2H), 3.20 (m, 3H), |
| | yl]amino}thieno[3,2- | | | 2.86 (m, 3H), 1.92 (m, 3H), 1.70 (m, 1H) |
| | c]pyridine-7-carboxamide | | | |
| 68 | 4-{(2- | 462.64 | 475 | D₂O added: 8.74 (s, 1H), 7.97 (s, 1H), |
| | phenylethyl)[(3S)- | | | 7.77 (dd, 1H), 7.59 (d, 1H), 7.56 (s, 1H), |
| | piperidin-3-yl]amino}- | | | 7.34 (m, 3H), 7.24 (m, 2H), 4.57 (m, |
| | 2-(3-thienyl)thieno[3,2- | | | 1H), 3.95 (m, 2H), 3.25 (m, 3H), |
| | c]pyridine-7- | | | 2.86 (m, 3H), 1.98 (m, 3H), 1.75 (m, 1H) |
| | carboxamide | | | |

### Example 69

### 4-{[trans-2-methylpiperidin-3-yl]amino}-2-phenylthieno[3,2-c]pyridine-7-carboxamide

Prepared in a similar fashion to Example 1 but using benzyl trans-3-amino-2-methylpiperidine-1-carboxylate (synthesis described below) as the starting material in step 7.

¹H NMR δ 9.47 (m, 1H), 9.04 (m, 1H), 8.59 (m, 1H), 8.45 (s, 1H), 8.25 (m, 1H), 7.71 (d, 2H), 7.56 (m, 1H), 7.45 (m, 2H), 7.35 (m, 1H), 4.34 (m, 1H), 3.42 (m, 1H), 3.22 (m, 1H), 2.82 (m, 1H), 2.07 (m, 1H), 1.83 (m, 2H), 1.59 (m, 1H), 1.26 (d, 3H). LCMS (ES, M+H=367).

tert-butyl ((1S)-1-acetyl-4-{[(benzyloxy)carbonyl]amino}butyl)carbamate. To a 3-necked flask containing N⁵-[(benzyloxy)carbonyl]-N²-(tert-butoxycarbonyl)-L-ornithine (36.6 g, 100 mmol) equipped with a magnetic stir bar and an addition funnel is added dry THF (100 mL).

The addition funnel is charged with MeLi (1.6M in ether; 275 mL; 440 mmol), which is subsequently added slowly (over 20 minutes) to the reaction mixture cooled to 0° C. This solution is then warmed to rt. After stirring for an additional 5 h, the reaction is quenched by pouring onto a stirred ice/water mixture. The aqueous mixture is extracted with EtOAc (3×100 mL). The combined organic layers are then washed with brine, dried over Na₂SO₄, filtered and concentrated in vacuo to yield a yellow oil (6.0 g, 98%). After purification using MPLC (SiO₂; 25-60% EtOAc/Hexanes), the product is isolated as a clear oil (5.2 g, 14%). ¹H NMR δ 7.35 (m, 5H), 7.25 (m, 2H), 5.00 (s, 2H), 3.85 (m, 1H), 2.98 (dd, 2H), 2.05 (s, 3H), 1.63 (m, 1H), 1.39 (s, 9H), 1.34 (m, 3H). LCMS (ES, M+H=365).

tert-butyl [trans-2-methylpiperidin-3-yl]carbamate. To a stirred solution of tert-yl

((1S)-1-acetyl-4-{[(benzyloxy)carbonyl]amino}butyl)carbamate (3.9 g, 10.7 mmol) in MeOH (200 mL) is added 10% Pd/C (0.1 mmol). The heterogeneous mixture is hydrogenated at atmospheric pressure for 3 days (or 40 psi overnight). The product is isolated as clear oil after filtration through diatomaceous earth and evaporation of the filtrate to give the title compound (2.3 g; 100%), which is used in the next step without purification. LCMS (ES, M+H=215).

benzyl trans-3-[(tert-butoxycarbonyl)amino]-2-methylpiperidine-1-carboxylate. To a stirred solution of tert-butyl [trans-2-methylpiperidin-3-yl]carbamate (2.3 g, 10.7 mmol) and diisopropylethylamine (2.1 mL, 12 mmol) dissolved in CH₂Cl₂ (40 mL) cooled to 0° C. is added benzyl chloroformate (1.7 mL, 12 mmol). The reaction mixture is then warmed to rt and stirred for an additional 1 h. The mixture is then diluted with CH₂Cl₂ and washed with 1N HCl and brine, dried over Na₂SO₄, filtered and concentrated in vacuo to yield a yellow oil. After purification using MPLC (SiO₂; 10-40% EtOAc/Hexanes), the title compound (trans diastereomer) is isolated as a crystalline solid (1.8 g). ¹H NMR δ 7.34 (m, 5H), 6.99 (d, 1H), 5.04 (s, 2H), 4.28 (dd, 1H), 3.83 (m, 1H), 3.37 (m, 1H), 2.86 (m, 1H), 1.77 (m, 2H), 1.46 (m, 1H), 1.36 (s, 9H), 1.33 (m, 1H), 1.11 (d, 3H). LCMS (ES, M+H=349). The cis diastereomer, benzyl cis-3-[(tert-butoxycarbonyl)amino]-2-methylpiperidine-1-carboxylate is also isolated pure (1.3 g). ¹H NMR δ 7.35 (m, 5H), 6.97 (d, 1H), 5.07 (s, 2H), 4.44 (m, 1H), 3.80 (m, 1H), 3.40 (m, 1H), 2.78 (m, 1H), 1.63 (m, 1H), 1.49 (m, 2H), 1.39 (s, 9H), 1.36 (m, 1H), 0.96 (d, 3H). LCMS (ES, M+H=349).

benzyl trans-3-amino-2-methylpiperidine-1-carboxylate. To a solution of benzyl trans-3-[(tert-butoxycarbonyl)amino]-2-methylpiperidine-1-carboxylate (1.8 g, 5.2 mmol) dissolved in MeOH (10 mL) is added HCl (4N in dioxane; 20 mL). After stirring for 1 h at rt, the reaction is concentrated in vacuo, redissolved in MeOH, and then concentrated in vacuo to yield the hydrochloride salt of the title compound as a clear crystalline solid (1.46 g, 100%). ¹H NMR δ 8.27 (br s, 3H), 7.39 (m, 3H), 7.35 (m, 1H), 7.32 (m, 1H), 5.09 (s, 2H), 4.36 (dd, 1H), 3.88 (m, 1H), 3.26 (m, 1H), 2.92 (m, 1H), 1.79 (m, 3H), 1.48 (m, 1H), 1.16 (d, 3H). LCMS (ES, M+H=249).

### Example 70

### 4-{[cis-2-methylpiperidin-3-yl]amino}-2-phenylthieno[3,2-c]pyridine-7-carboxamide

Prepared in a similar fashion to Example 1 but using benzyl cis-3-amino-2-methylpiperidine-1-carboxylate (described below) as the starting material in step 7. ¹H NMR δ 9.79 (m, 1H), 8.95 (m, 2H), 8.51 (s, 1H), 8.15 (m, 1H), 7.78 (d, 2H), 7.49 (m, 3H), 7.38 (m, 1H), 4.73 (m, 1H), 3.71 (m, 1H), 3.28 (m, 1H), 3.00 (m, 1H), 1.95 (m, 2H), 1.82 (m, 1H), 1.69 (m, 1H), 1.30 (d, 3H). LCMS (ES, M+H=367).

benzyl cis-3-amino-2-methylpiperidine-1-carboxylate. To a solution of benzyl cis-3-[(tert-butoxycarbonyl)amino]-2-methylpiperidine-1-carboxylate (1.2 g, 3.4 mmol) dissolved in MeOH (10 mL) is added HCl (4N in dioxane; 20 mL). After stirring for 1 h at rt, the reaction is concentrated in vacuo, redissolved in MeOH, and then concentrated in vacuo to yield the hydrochloride salt of the title compound as a clear crystalline solid (0.97 g, 100%). ¹H NMR 8.39 (br s, 3H), 7.36 (m, 3H), 7.33 (m, 2H), 5.09 (s, 2H), 4.61 (dd, 1H), 3.83 (m, 1H), 3.26 (m, 1H), 2.86 (m, 1H), 1.72 (m, 3H), 1.41 (m, 1H), 1.10 (d, 3H). LCMS (ES, M+H=249).

The following examples 71-74 are prepared in a similar fashion.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 71 | 2-(3-fluorophenyl)-4- | 384.48 | 385 | 9.50 (s, 1H), 9.11 (s, 1H), 8.71 (s, 1H), |
| | {[trans-2- | | | 8.55 (s, 1H), 8.30 (s, 1H), 7.60 (m, |
| | methylpiperidin-3- | | | 5H), 7.27 (t, 1H), 4.42 (s, 1H), |
| | yl]amino}thieno[3,2- | | | 3.31 (m, 2H), 2.94 (m, 1H), 2.11 (m, 1H), |
| | c]pyridine-7-carboxamide | | | 1.91 (m, 2H), 1.68 (m, 1H), 1.33 (d, |
| | | | | 3H). |
| 72 | 4-{[trans-2- | 372.52 | 373 | 9.45 (s, 1H), 9.03 (s, 1H), 8.51 (s, 1H), |
| | methylpiperidin-3- | | | 8.36 (s, 1H), 8.30 (s, 1H), 7.91 (s, 1H), |
| | yl]amino}-2-(3- | | | 7.74 (d, 1H), 7.60 (s, 1H), 7.50 (d, |
| | thienyl)thieno[3,2- | | | 2H), 4.40 (s, 1H), 3.31 (m, 2H), |
| | c]pyridine-7-carboxamide | | | 2.92 (m, 1H), 2.09 (m, 1H), 1.90 (m, 2H), |
| | | | | 1.66 (m, 1H), 1.32 (d, 3H). |
| 73 | 2-(4-fluorophenyl)-4- | 384.48 | 385 | 9.55 (s, 1H), 9.15 (s, 1H), 8.71 (s, 1H), |
| | {[trans-2- | | | 8.52 (s, 1H), 8.41 (s, 1H), 7.82 (t, 2H), |
| | methylpiperidin-3- | | | 7.67 (s, 1H), 7.38 (t, 2H), 4.37 (s, 1H), |
| | yl]amino}thieno[3,2- | | | 3.31 (d, 2H), 2.94 (m, 1H), 2.14 (m, 1H), |
| | c]pyridine-7-carboxamide | | | 1.91 (m, 2H), 1.66 (m, 1H), 1.34 (d, 3H). |
| 74 | 2-(2-fluorophenyl)-4- | 384.48 | 385 | 9.75 (d, 1H), 9.22 (d, 1H), 8.87 (s, |
| | {[trans-2- | | | 1H), 8.55 (s, 1H), 8.45 (m, 1H), |
| | methylpiperidin-3- | | | 7.91 (t, 2H), 7.67 (s, 1H), 7.43 (m, 3H), |
| | yl]amino}thieno[3,2- | | | 4.39 (d, 1H), 3.26 (d, 2H), 2.89 (m, |
| | c]pyridine-7-carboxamide | | | 1H), 2.18 (d, 1H), 1.92 (s, 2H), |
| | | | | 1.66 (m, 1H), 1.36 (d, 3H). |

The following examples 75-76 are prepared by chiral preparatory HPLC separation of Example 69.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 75 | 4-{[(2S,3R)-2- | 366.49 | 367 | 9.47 (m, 1H), 9.04 (m, 1H), 8.59 (m, |
| | methylpiperidin-3- | | | 1H), 8.45 (s, 1H), 8.25 (m, 1H), |
| | yl]amino}-2- | | | 7.71 (d, 2H), 7.56 (m, 1H), 7.45 (m, 2H), |
| | phenylthieno[3,2- | | | 7.35 (m, 1H), 4.34 (m, 1H), 3.42 (m, |
| | c]pyridine-7- | | | 1H), 3.22 (m, 1H), 2.82 (m, 1H), |
| | carboxamide | | | 2.07 (m, 1H), 1.83 (m, 2H), 1.59 (m, 1H), |
| | | | | 1.26 (d, 3H). |
| 76 | 4-{[(2R,3S)-2- | 366.49 | 367 | 9.47 (m, 1H), 9.04 (m, 1H), 8.59 (m, |
| | methylpiperidin-3- | | | 1H), 8.45 (s, 1H), 8.25 (m, 1H), |
| | yl]amino}-2- | | | 7.71 (d, 2H), 7.56 (m, 1H), 7.45 (m, 2H), |
| | phenylthieno[3,2- | | | 7.35 (m, 1H), 4.34 (m, 1H), 3.42 (m, |
| | c]pyridine-7- | | | 1H), 3.22 (m, 1H), 2.82 (m, 1H), |
| | carboxamide | | | 2.07 (m, 1H), 1.83 (m, 2H), 1.59 (m, 1H), |
| | | | | 1.26 (d, 3H). |

The following examples 77-78 are prepared by chiral preparatory HPLC separation of Example 72.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 77 | 4-{[(2S,3R)-2- | 372.52 | 373 | 9.45 (s, 1H), 9.03 (s, 1H), 8.51 (s, 1H), |
| | methylpiperidin-3- | | | 8.36 (s, 1H), 8.30 (s, 1H), 7.91 (s, 1H), |
| | yl]amino}-2-(3- | | | 7.74 (d, 1H), 7.60 (s, 1H), 7.50 (d, |
| | thienyl)thieno[3,2- | | | 2H), 4.40 (s, 1H), 3.31 (m, 2H), |
| | c]pyridine-7- | | | 2.92 (m, 1H), 2.09 (m, 1H), 1.90 (m, 2H), |
| | carboxamide | | | 1.66 (m, 1H), 1.32 (d, 3H). |
| 78 | 4-{[(2R,3S)-2- | 372.52 | 373 | 9.45 (s, 1H), 9.03 (s, 1H), 8.51 (s, 1H), |
| | methylpiperidin-3- | | | 8.36 (s, 1H), 8.30 (s, 1H), 7.91 (s, 1H), |
| | yl]amino}-2-(3- | | | 7.74 (d, 1H), 7.60 (s, 1H), 7.50 (d, |
| | thienyl)thieno[3,2- | | | 2H), 4.40 (s, 1H), 3.31 (m, 2H), |
| | c]pyridine-7- | | | 2.92 (m, 1H), 2.09 (m, 1H), 1.90 (m, 2H), |
| | carboxamide | | | 1.66 (m, 1H), 1.32 (d, 3H). |

### Example 79

### 4-{methyl[trans-2-methylpiperidin-3-yl]amino}-2-phenylthieno[3,2-c]pyridine-7-carboxamide

Prepared in a similar fashion to Example 1 but using benzyl trans-2-methyl-3-(methylamino)piperidine-1-carboxylate (synthesis described below) as the starting material in step 7. ¹H NMR δ 9.57 (m, 1H), 8.92 (m, 1H), 8.61 (s, 1H), 8.14 (br s, 1H), 7.97 (s, 1H), 7.87 (d, 2H), 7.50 (m, 3H), 7.40 (m, 1H), 4.82 (m, 1H), 3.55 (m, 1H), 3.29 (s, 3H), 3.23 (m, 1H), 2.94 (m, 1H), 1.97 (m, 4H), 1.21 (d, 3H). LCMS (ES, M+H=381).

benzyl trans-3-[(tert-butoxycarbonyl)(methyl)amino]-2-methylpiperidine-1-carboxylate.

To a solution of benzyl trans-3-[(tert-butoxycarbonyl)amino]-2-methylpiperidine-1-carboxylate (0.10 g, 0.29 mmol) in 10 mL dry THF under a N₂ atmosphere is added sodium hydride (60% in mineral oil; 8 mg, 0.32 mmol). This solution is stirred for 30 minutes and then methyl iodide (0.017 mL, 0.287 mmol) is added. The reaction mixture is stirred for two hours and 10 mL of MeOH is added slowly to quench the reaction. The contents are CIV and the residue is dissolved in 30 mL CH₂Cl₂ and washed with water (2×). The organic layer is CIV to yield 0.16 g of the title product. ¹H NMR δ 1.14 (d, 3H) 1.38 (s, 9H) 1.54 (m, 1H) 1.68 (m, 3H) 2.73 (s, 3H) 3.06 (m, 1H) 3.74 (m, 1H) 3.84 (m, 1H) 4.04 (m, 1H) 5.07 (s, 2H) 7.34 (m, 5H). LCMS (ES, M+H=363).

benzyl trans-2-methyl-3-(methylamino)piperidine-1-carboxylate. To a solution of benzyl trans-3-[(tert-butoxycarbonyl)(methyl)amino]-2-methylpiperidine-1-carboxylate (0.16 g 0.45 mmol) dissolved in MeOH (4 mL) is added HCl (4N in dioxane; 4 mL). After stirring for 2 h at rt, the reaction is concentrated in vacuo, redissolved in MeOH, and then concentrated in vacuo to yield the hydrochloride salt of the title compound as an oily crystalline solid (0.12 g). LCMS (ES, M+H=263).

### Example 80

### 4-{[trans-2-(2-hydroxyethyl)piperidin-3-yl]amino}-2-phenylthieno[3,2-c]pyridine-7-carboxamide

Prepared in a similar fashion to Example 1 but using benzyl trans-3-amino-2-(2-hydroxyethyl)piperidine-1-carboxylate (synthesis described below) as the starting material in step 7. ¹H NMR δ 9.57 (m, 1H), 8.92 (m, 1H), 8.61 (s, 1H), 8.14 (br s, 1H), 7.97 (s, 1H), 7.87 (d, 2H), 7.50 (m, 3H), 7.40 (m, 1H), 4.82 (m, 1H), 3.55 (m, 1H), 3.29 (s, 3H), 3.23 (m, 1H), 2.94 (m, 1H), 1.97 (m, 4H), 1.21 (d, 3H). LCMS (ES, M+H=381).

benzyl trans-3-[(tert-butoxycarbonyl)amino]-2-(2-ethoxy-2-oxoethyl)piperidine-1-carboxylate. ethyl {3-[(tert-butoxycarbonyl)amino]piperidin-2-yl}acetate (prepared following the procedure described in: Tetrahedron Lett, 1993, 34, 3593-3594) (0.45 g, 1.6 mmol) is dissolved in 10 mL CH₂Cl₂ under dry and N₂ purged conditions. 0.28 mL (1.6 mmol) DIEA is added, and then 0.22 mL (1.59 mmol) benzyl chloroformate. This solution is stirred for 30 min. The reaction mixture is then extracted with water, then brine. The organic layer is concentrated and the residue purified by MPLC; 0-50% EtOAc/Hexanes. The title compound elutes at 43-48%, 0.41 g. LCMS (ES, M+H=421).

benzyl trans-3-amino-2-(2-hydroxyethyl)piperidine-1-carboxylate. Benzyl trans-3-[(tert-butoxycarbonyl)amino]-2-(2-ethoxy-2-oxoethyl)piperidine-1-carboxylate (0.41 g) is dissolved in 9 mL THF and 1 mL MeOH under dry and N₂ purged conditions using dry solvents. To this is added 0.073 g NaBH₄, and stirred for 16 hr. Gas evolution is observed upon addition of NaBH₄. The reaction is diluted with 25 mL water, and extracted with CH₂Cl₂. The organic extracts are concentrated, and the residue is dissolved in 4 mL 4N HCl in Dioxane. This solution is stirred for 16 hr and upon removal of solvent under high vacuum yields the title compound, 0.20 g. LCMS (ES, M+H=279).

### Example 81

### 4-{[(3S)-5-benzylpiperidin-3-yl]amino}-2-phenylthieno[3,2-c]pyridine-7-carboxamide

Prepared in a similar fashion to Example 1 but using benzyl (3S)-5-benzylpiperidin-3-amine (synthesis described below) as the starting material in step 7. ¹H NMR δ 8.64 (s, 1H), 8.19 (m, 1H), 8.04 (m, 2H), 7.59 (m, 2H), 7.56 (m, 2H), 7.43 (m, 3H), 7.30 (m, 3H), 7.22 (m, 2H), 3.99 (m, 2H), 3.72 (m, 3H), 3.37 (d, 1H), 2.91 (m, 1H), 2.70 (m, 1H), 1.99 (m, 1H), 1.69 (m, 1H). LCMS (ES, M+H=443).

dimethyl 4-benzyl-N-(tert-butoxycarbonyl)-L-glutamate. To a solution of lithium hexamethyldisilylazide (50.0 mL, 50.0 mmol, 1M in THF) at −78° C. is added drop wise a solution of dimethyl N-(tert-butoxycarbonyl)-L-glutamate (6.55 g, 23.8 mmol) in 30 mL THF. The resulting solution is stirred for thirty minutes followed by the addition of benzyl bromide (5.65 mL, 47.6 mmol). The reaction mixture is then stirred for one hour at −78° C., after which time LCMS indicated complete conversion to product. The reaction mixture is quenched with water (50 mL) and extracted with EtOAc (3×100 mL). The organic layers are dried over magnesium sulfate, filtered, and concentrated under reduced pressure to yield the title compound as a white solid (5.68 grams, 65% yield) after purification by flash column chromatography (100% hexanes to 100% EtOAc). LCMS (ES, M+Na=388).

tert-butyl [(1S)-3-benzyl-4-hydroxy-1-(hydroxymethyl)butyl]carbamate. To a solution containing dimethyl 4-benzyl-N-(tert-butoxycarbonyl)-L-glutamate (5.68 g, 15.5 mmol) and calcium chloride (6.88 g, 62.0 mmol) in 60 mL each of EtOH and THF is added at 0° C. NaBH₄ (4.69 g, 124 mmol) in portions. The reaction mixture is warmed to rt and stirred for 12 hours until LCMS indicates complete conversion to product. The reaction mixture is then quenched with saturated sodium bicarbonate (2×100 mL) and water (2×100 mL) followed by extraction with EtOAc (3×100 mL). The combined organic layers are dried over magnesium sulfate, filtered, and concentrated under reduced pressure to afford the title compound that is used directly in the next reaction. LCMS (ES, M+H−BOC group=210).

(2S)-4-benzyl-2-[(tert-butoxycarbonyl)amino]-5-[(methylsulfonyl)oxy]pentyl methanesulfonate. A solution of tert-butyl [(1S)-3-benzyl-4-hydroxy-1-(hydroxymethyl)butyl]carbamate (4.42 g, 14.3 mmol) in 100 mL CH₂Cl₂ is cooled to 0° C. whereupon TEA (7.97 mL, 57.2 mmol) is added followed by methanesulfonyl chloride (3.32 mL, 42.9 mmol). The reaction mixture is stirred for one hour at 0° C., diluted with CH₂Cl₂, washed with saturated sodium bicarbonate, dried over magnesium sulfate, filtered, and concentrated in vacuo to afford the title compound which is used directly in the next reaction. LCMS (ES, M+Na=488).

tert-butyl [(3S)-1,5-dibenzylpiperidin-3-yl]carbamate. To a solution of (2S)-4-benzyl-2-[(tert-butoxycarbonyl)amino]-5-[(methylsulfonyl)oxy]pentyl methanesulfonate (14.3 mmol) is added 30 mL of benzylamine. The reaction mixture is heated to 70° C. for approximately 24 hours after which the mixture is cooled to rt and poured into 1 N NaOH (100 mL). The mixture is extracted with hexanes (4×100 mL), the organic layers dried over magnesium sulfate, filtered, and CIV. The resulting oil is purified by flash column chromatography (100% hexanes to 100% EtOAc) to afford the title compound. LCMS (ES, M+H−BOC group=281).

tert-butyl [(3S)-5-benzylpiperidin-3-yl]carbamate. To a solution of tert-butyl [(3S)-1,5-dibenzylpiperidin-3-yl]carbamate (3.28 g, 8.62 mmol) in 15 mL EtOH is added 10% Pd/C (900 mg) under nitrogen. The reaction mixture is treated with 50 psi of hydrogen on a Parr apparatus for 24 hours. The reaction mixture is filtered over diatomaceous earth, rinsed with copious amounts of MeOH, and the filtrate concentrated under reduced pressure to afford the title compound that is used directly in the next reaction. LCMS (ES, M+H=291).

(3S)-5-benzylpiperidin-3-amine. To a solution containing tert-butyl [(3S)-5-benzylpiperidin-3-yl]carbamate (1.59 g, 5.46 mmol) dissolved in a minimal amount of MeOH is added 4N HCl in dioxane (5.0 mL). The resulting solution is stirred at rt for thirty minutes. The reaction mixture is then concentrated under reduced pressure to yield the title compound. LCMS (ES, M+H=191).

Example 82 is prepared in a similar fashion to Example 81 using appropriate starting materials.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 82 | 4-{[(3S)-5- | 448.61 | 449 | 8.63 (s, 1H), 8.17 (m, 1H), 8.03 (m, 3H), |
| | benzylpiperidin-3- | | | 7.84 (s, 1H), 7.73 (m, 1H), 7.60 (m, 1H), |
| | yl]amino}-2-(3- | | | 7.37 (m, 2H), 7.27 (m, 2H), 7.20 (m, |
| | thienyl)thieno[3,2- | | | 2H), 3.99 (m, 2H), 3.83 (m, 2H), |
| | c]pyridine-7- | | | 3.38 (m, 2H), 2.94 (m, 1H), 2.72 (m, 1H), |
| | carboxamide | | | 1.95 (m, 1H), 1.68 (m, 1H) |

### Example 83

### 4-[(2,6-dimethylpiperidin-3-yl)amino]-2-phenylthieno[3,2-c]pyridine-7-carboxamide

2,6-dimethylpiperidin-3-amine. To a high-pressure vessel containing 2,6-dimethylpyridin-3-amine (2.08 g, 17.0 mmol) is added water and 12 N HCl (10 mL each) followed by platinum (IV) oxide (500 mg, 2.20 mmol) under nitrogen. The high-pressure vessel is then evacuated under reduced pressure and placed on a Parr hydrogenation apparatus at 50 psi for 48 hours. The mixture is evacuated under nitrogen, filtered over a bed of diatomaceous earth, and rinsed with copious amounts of MeOH. The collected filtrate is concentrated in vacuo to afford the title compound that is used directly in the next reaction as a mixture of isomers. LCMS (ES, M+H=129).

2-bromo-4-[(2,6-dimethylpiperidin-3-yl)amino]thieno[3,2-c]pyridine-7-carbonitrile. To 2,6-dimethylpiperidin-3-amine (1.23 g, 4.50 mmol) dissolved in NMP (10 mL) is added potassium carbonate (1.87 g, 13.5 mmol) and 2-bromo-4-chlorothieno[3,2-c]pyridine-7-carbonitrile (1.23 g, 4.50 mmol). The resulting mixture is heated to 80° C. and stirred for twelve hours or until LCMS indicates complete conversion to product. The mixture is then diluted with water (100 mL) and the resulting solid is filtered, washed with water (20 mL) and dried under reduced pressure for up to eight hours. LCMS (ES, M+H=366). 4-[(2,6-dimethylpiperidin-3-yl)amino]-2-phenylthieno[3,2-c]pyridine-7-carbonitrile. To a solution containing 2-bromo-4-[(2,6-dimethylpiperidin-3-yl)amino]thieno[3,2-c]pyridine-7-carbonitrile (400 mg, 1.09 mmol), phenylboronic acid (200 mg, 1.64 mmol), cesium carbonate (1.06 g, 3.27 mmol), and dioxane/water (2 mL/1 mL) is added Pd(PPh₃)₄ (126 mg, 0.109 mmol). The reaction is heated to 80° C. for one hour whereupon the reaction is cooled to rt, filtered, and purified using silica gel chromatography (100% CH₂Cl₂ to 20% MeOH/CH₂Cl₂/3% NH₄OH) to afford the title compound. LCMS (ES, M+H=363).

4-[(2,6-dimethylpiperidin-3-yl)amino]-2-phenylthieno[3,2-c]pyridine-7-carboxamide. To a flask containing 4-[(2,6-dimethylpiperidin-3-yl)amino]-2-phenylthieno[3,2-c]pyridine-7-carbonitrile is added 5.00 mL of 12 N HCl. The reaction mixture is stirred at rt and monitored by LCMS. Additional 12 N HCl is added every twelve hours to afford complete conversion to the desired product. Upon completion, the reaction mixture is diluted with MeOH and concentrated under reduced pressure to yield the product, which is purified by preparatory HPLC (5%-95% H₂O/MeCN/0.1% TFA) to afford the title compound as a mixture of isomers. Analytical data provided for major isomer present in mixture: ¹H NMR δ 9.96 (m, 1H), 9.19 (m, 1H), 9.02 (m, 1H), 8.50 (s, 1H), 8.21 (m, 1H), 7.79 (m, 2H), 7.49 (m, 2H), 7.35 (m, 2H), 4.74 (m, 1H), 3.67 (m, 1H), 3.27 (m, 1H), 1.84 (m, 4H), 1.36 (m, 6H). LCMS (ES, M+H=3381).

The following examples 84-94 are prepared in a similar fashion using the appropriate starting materials.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 84 | 4-[(2,6-dimethylpiperidin- | 398.5 | 399 | 9.93 (m, 1H), 9.15 (m, 1H), 9.04 (m, 1H), |
| | 3-yl)amino-2-(4- | | | 8.51 (s, 1H), 8.21 (m, 1H), 7.85 (m, 2H), |
| | fluorophenyl)thieno[3,2- | | | 7.57 (m, 1H), 7.34 (m, 1H), 7.15 (m, 1H), |
| | c]pyridine-7-carboxamide | | | 4.72 (m, 1H), 3.67 (m, 1H), 3.26 (m, 1H), |
| | | | | 1.83 (m, 4H), 1.34 (m, 6H) |
| 85 | 4-[(2,6- | 398.5 | 399 | 9.87 (m, 1H), 9.21 (m, 1H), 8.96 (m, |
| | dimethylpiperidin-3- | | | 1H), 8.53 (s, 1H), 8.16 (m, 1H), |
| | yl)amino]-2-(3- | | | 7.56 (m, 3H), 7.22 (m, 2H), 4.76 (m, 1H), |
| | fluorophenyl)thieno[3,2- | | | 3.65 (m, 1H), 3.25 (m, 1H), 1.78 (m, |
| | c]pyridine-7-carboxamide | | | 4H), 1.31 (m, 6H) |
| 86 | 4-[(2,6- | 386.54 | 387 | 9.90 (m, 1H), 9.01 (m, 2H), 8.48 (s, |
| | dimethylpiperidin-3- | | | 1H), 8.19 (m, 1H), 7.87 (s, 1H), |
| | yl)amino]-2-(3- | | | 7.72 (m, 1H), 7.54 (m, 2H), 4.71 (m, 1H), |
| | thienyl)thieno[3,2- | | | 3.67 (m, 1H), 3.26 (m, 1H), 1.84 (m, |
| | c]pyridine-7-carboxamide | | | 4H), 1.36 (m, 6H) |
| 87 | 2-phenyl-4-{[6- | 420.46 | 421 | 8.83 (m, 1H), 8.54 (m, 1H), 8.25 (m, |
| | (trifluoromethyl)piperidin- | | | 1H), 7.76 (m, 2H), 7.51 (m, 3H), |
| | 3-yl]amino}thieno[3,2- | | | 7.40 (m, 1H), 4.65 (m, 1H), 4.44 (m, 1H), |
| | c]pyridine-7-carboxamide | | | 3.68 (m, 1H), 3.53 (m, 1H), 2.14 (m, |
| | | | | 2H), 2.02 (m, 2H) |
| 88 | 4-[(4-methylpiperidin-3- | 366.49 | 367 | 8.67 (s, 1H), 8.24 (s, 1H), 8.06 (m, |
| | yl)amino]-2- | | | 2H), 7.88 (m, 3H), 7.62 (m, 1H), |
| | phenylthieno[3,2- | | | 7.51 (m, 2H), 7.42 (m, 1H), 4.12 (m, 1H), |
| | c]pyridine-7-carboxamide | | | 3.98 (m, 1H), 3.52 (m, 1H), 3.41 (m, |
| | | | | 1H), 3.18 (m, 1H), 2.10 (m, 1H), |
| | | | | 1.92 (m, 2H), 1.06 (d, 3H) |
| 89 | 4-[(4-methylpiperidin-3- | 372.52 | 373 | 8.65 (s, 1H), 8.17 (m, 1H), 8.01 (m, |
| | yl)amino]-2-(3- | | | 3H), 7.72 (m, 2H), 7.67 (m, 1H), |
| | thienyl)thieno[3,2- | | | 7.56 (m, 1H), 4.08 (m, 1H), 3.94 (m, 1H), |
| | c]pyridine-7-carboxamide | | | 3.53 (m, 1H), 3.38 (m, 1H), 3.17 (m, |
| | | | | 1H), 2.09 (m, 1H), 1.89 (m, 1H), |
| | | | | 1.72 (m, 1H), 1.06 (d, 3H) |
| 90 | 2-(3-fluorophenyl)-4-[(4- | 384.48 | 385 | 8.68 (s, 1H), 8.17 (m, 1H), 8.01 (m, 2H), |
| | methylpiperidin-3- | | | 7.91 (s, 1H), 7.77 (d, 1H), 7.66 (m, 1H), |
| | yl)amino]thieno[3,2- | | | 7.56 (m, 2H), 7.26 (m, 1H), 3.97 (m, 2H), |
| | c]pyridine-7-carboxamide | | | 3.53 (m, 1H), 3.40 (m, 1H), 3.19 (m, 1H), |
| | | | | 2.10 (m, 1H), 1.93 (m, 1H), 1.74 (m, 1H), |
| | | | | 1.06 (d, 3H) |
| 91 | 2-(3-fluorophenyl)-4-[(6- | 384.48 | 385 | 9.38 (m, 1H), 9.08 (m, 1H), 8.88 (m, |
| | methylpiperidin-3- | | | 1H), 8.55 (s, 1H), 8.15 (m, 1H), |
| | yl)amino]thieno[3,2- | | | 7.56 (m, 4H), 7.24 (m, 1H), 4.60 (m, 1H), |
| | c]pyridine-7-carboxamide | | | 3.49 (m, 1H), 3.32 (m, 2H), 1.87 (m, |
| | | | | 4H), 1.33 (d, 3H) |
| 92 | 2-(4-fluorophenyl)-4-[(6- | 384.48 | 385 | 9.59 (m, 1H), 9.16 (m, 1H), 8.89 (m, |
| | methylpiperidin-3- | | | 1H), 8.52 (s, 1H), 8.18 (m, 1H), |
| | yl)amino]thieno[3,2- | | | 7.81 (m, 2H), 7.57 (m, 1H), 7.35 (m, 2H), |
| | c]pyridine-7-carboxamide | | | 4.62 (m, 1H), 3.46 (m, 1H), 3.28 (m, |
| | | | | 2H), 1.86 (m, 4H), 1.33 (d, 3H) |
| 93 | 4-[(6-methylpiperidin-3- | 372.52 | 373 | 8.97 (m, 1H), 8.52 (m, 1H), 8.00 (m, |
| | yl)amino]-2-(3- | | | 1H), 7.84 (m, 1H), 7.73 (m, 1H), |
| | thienyl)thieno[3,2- | | | 7.49 (m, 2H), 4.56 (m, 1H), 4.10 (br s, 2H), |
| | c]pyridine-7-carboxamide | | | 3.47 (m, 1H), 3.29 (m, 2H), 1.89 (m, |
| | | | | 4H), 1.32 (d, 3H) |
| 94 | 4-[(6-methylpiperidin-3- | 366.49 | 367 | 9.84 (m, 1H), 9.41 (m, 1H), 9.18 (m, |
| | yl)amino]-2- | | | 1H), 8.53 (s, 1H), 8.40 (m, 1H), |
| | phenylthieno[3,2- | | | 7.72 (m, 3H), 7.51 (m, 3H), 4.72 (m, 1H), |
| | c]pyridine-7-carboxamide | | | 3.21 (m, 3H), 1.89 (m, 4H), 1.34 (m, |
| | | | | 3H) |

### Example 95

### 2-{3-[(dimethylamino)methyl]phenyl}-4-[(3S)-piperidin-3-ylamino]thieno[3,2-c]pyridine-7-carboxamide

tert-butyl (3S)-3-{[7-cyano-2-(3-formylphenyl)-1-benzothien-4-yl]amino}piperidine-1-carboxylate. A mixture of tert-butyl (3S)-3-[(7-cyano-2-bromothieno[3,2-c]pyridin-4-yl)amino]piperidine-1-carboxylate (500 mg, 1.1 mmol), 3-formylphenylboronic acid (257 mg, 1.7 mmol), cesium carbonate (1.12 g, 3.4 mmol) and Pd(PPh₃)₄ (198 mg, 0.17 mmol) are heated to 80° C. in dioxane (8.2 mL) and H₂O (2.7 mL). After 30 min, the solution is cooled to rt, the water layer is removed via pipette and the solution is concentrated in vacuo. The residue is purified by MPLC (SiO₂; 20-50% EtOAc/hexanes) to yield the title compound (185 mg, 35%). LCMS (ES, M+H=463).

tert-butyl (3S)-3-[(7-cyano-2-{4-[(dimethylamino)methyl]phenyl}-1-benzothien-4-yl)amino]piperidine-1-carboxylate. A solution of tert-butyl (3S)-3-{[7-cyano-2-(3-formylphenyl)-1-benzothien-4-yl]amino}piperidine-1-carboxylate (50 mg, 0.11 mmol) and dimethylamine (0.54 mL of a 2 M solution in THF, 1.1 mmol) are stirred in ethylene glycol dimethyl ether (0.54 mL) at rt. Acetic Acid (2 drops) is added, followed by NaBH(OAc)₃ (92 mg, 0.43 mmol). The solution is heated to 80° C. for 30 min. The solution is then cooled to rt. Water (2 drops) is added, followed by 1 M NaOH (1 mL). The product is extracted with EtOAc. The organic extracts are washed with brine, dried over MgSO₄ and concentrated in vacuo to yield the title compound, which is used directly in the next step. LCMS (ES, M+H 492).

2-{3-[(dimethylamino)methyl]phenyl}-4-[(3S)-piperidin-3-ylamino]thieno[3,2-c]pyridine-7-carboxamide tert-Butyl (3S)-3-[(7-cyano-2-{4-[(dimethylamino)methyl]phenyl}-1-benzothien-4-yl)amino]piperidine-1-carboxylate is dissolved in 12 M HCl (4 mL) and kept at rt for 17 h. The solution is concentrated in vacuo, and azeotroped with MeOH to yield the title compound as the hydrochloride salt (48 mg, 98%). ¹H NMR δ 10.62 (br s, 1H), 9.70 (br s, 1H), 9.07 (m, 2H), 8.57 (s, 1H), 8.44 (br s, 1H), 7.96 (s, 1H), 7.90 (m, 1H), 7.60 (m, 3H), 4.65 (m, 1H), 4.35 (d, 2H), 3.47 (m, 1H), 3.20 (m, 2H), 3.01 (m, 1H), 2.74 (s, 3H), 2.73 (s, 3H), 2.03 (m, 2H), 1.81 (m, 2H). LCMS (ES, M+H=410).

The following examples 96-111 are prepared in a similar fashion from the appropriate materials.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 96 | 2-{4-[(dimethylamino)methyl]phenyl}- | 409.56 | 410 | 10.44 (br s, 1H), 9.44 (br s, 1H), 8.95 (br s, 1H), |
| | 4- | | | 8.76 (br s, 1H), 8.55 (s, 1H), 8.21 (br s, 1H), |
| | [(3S)-piperidin-3- | | | 7.85 (d, 2H), 7.66 (d, 2H), 7.52 (br s, 1H), 4.59 (m, |
| | ylamino] thieno[3,2- | | | 1H), 4.30 (d, 2H), 3.33 (m, 1H), 3.18 (m, 1H), |
| | c]pyridine-7- | | | 3.10 (m, 1H), 3.00 (m, 1H), 2.73 (s, 3H), 2.72 (s, |
| | carboxamide | | | 3H), 2.01 (m, 2H), 1.77 (m, 2H) |
| 97 | 4-[(3S)-piperidin-3- | 449.62 | 450 | 10.34 (br s, 1H), 9.54 (br s, 1H), 8.99 (br |
| | ylamino]-2-[4- | | | s, 1H), 8.85 (br s, 1H), 8.56 (s, 1H), |
| | (piperidin-1- | | | 8.26 (br s, 1H), 7.84 (d, 2H), 7.70 (d, 2H), |
| | ylmethyl)phenyl]thieno[3,2- | | | 7.56 (br s, 1H), 4.61 (m, 1H), 4.28 (d, |
| | c]pyridine-7- | | | 2H), 3.45 (m, 1H), 3.31 (m, 2H), |
| | carboxamide | | | 3.16 (m, 1H), 3.02 (m, 1H), 2.85 (m, 2H), |
| | | | | 2.01 (m, 2H), 1.77 (m, 8H), 1.36 (m, |
| | | | | 1H) |
| 98 | 2-[4-(morpholin-4- | 451.59 | 452 | 11.53 (br s, 1H), 9.75 (br s, 1H), |
| | ylmethyl)phenyl]-4- | | | 9.11 (m, 2H), 8.57 (s, 1H), 8.44 (br s, 1H), |
| | [(3S)-piperidin-3- | | | 7.86 (d, 2H), 7.76 (d, 2H), 7.65 (br s, |
| | ylamino]thieno[3,2- | | | 1H), 4.66 (m, 1H), 4.36 (m, 2H), |
| | c]pyridine-7- | | | 3.96-2.95 (m, 12H), 2.02 (m, 2H), 1.81 (m, |
| | carboxamide | | | 2H) |
| 99 | 4-[(3S)-piperidin-3- | 449.62 | 450 | 9.94 (br s, 1H), 9.28 (br s, 1H), 8.91 (br |
| | ylamino]-2-[3- | | | s, 1H), 8.56 (m, 2H), 8.16 (br s, 1H), |
| | (piperidin-1- | | | 7.92 (s, 1H), 7.87 (m, 1H), 7.58 (m, 3H), |
| | ylmethyl)phenyl]thieno[3,2- | | | 4.55 (m, 1H), 4.34 (m, 2H), |
| | c]pyridine-7- | | | 3.50-2.60 (m, 8H), 2.00 (m, 2H), 1.80-1.50 (m, |
| | carboxamide | | | 8H) |
| 100 | 2-[3-(morpholin-4- | 451.59 | 452 | 10.40 (br s, 1H), 9.05 (br s, 1H), 8.83 (br |
| | ylmethyl)phenyl]-4- | | | s, 1H), 8.57 (s, 1H), 8.41 (br s, 1H), |
| | [(3S)-piperidin-3- | | | 8.06 (br s, 1H), 7.92 (s, 1H), 7.87 (m, 1H), |
| | ylamino]thieno[3,2- | | | 7.65-7.50 (m, 3H), 4.55 (m, 1H), |
| | c]pyridine-7- | | | 4.41 (m, 2H), 3.97 (m, 2H), 3.75-2.90 (m, |
| | carboxamide | | | 10H), 2.00 (m, 2H), 1.75 (m, 2H) |
| 101 | 2-(3-{[4-(2-methoxy- | 508.69 | 509 | 9.21 (br s, 1H), 8.86 (br s, 1H), 8.56 (s, |
| | ethyl)piperazin-1-yl]methyl}phenyl)- | | | 1H), 8.48 (br s, 1H), 8.20-7.70 (m, 4H), |
| | 4-[(3S)-piperidin-3-ylamino] | | | 7.65-7.30 (m, 3H), 4.56 (m, 2H), |
| | thieno[3,2c]pyridine-7- | | | 3.75-2.88 (m, 17H), 3.28 (s, 3H), 2.00 (m, |
| | carboxamide | | | 2H), 1.75 (m, 2H) |
| 102 | 2-{3- | 395.53 | 396 | 9.52 (br s, 1H), 9.15 (m, 2H), 9.01 (br s, |
| | [(methylamino)methyl]phenyl}- | | | 1H), 8.75 (br s, 1H), 8.57 (s, 1H), |
| | 4-[(3S)- | | | 8.26 (br s, 1H), 7.91 (s, 1H), 7.86 (m, 1H), |
| | piperidin-3- | | | 7.56 (m, 3H), 4.61 (m, 1H), 4.18 (m, |
| | ylamino]thieno[3,2- | | | 2H), 3.46 (m, 1H), 3.23-2.93 (m, 3H), |
| | c]pyridine-7- | | | 2.58 (t, 3H), 2.02 (m, 2H), 1.78 (m, 2H) |
| | carboxamide | | | |
| 103 | 2-(4-{[4-(2- | 508.69 | 509 | 12.75-11.20 (m, 1H), 9.75 (br s, 1H), |
| | methoxyethyl)piperazin- | | | 9.43 (br s, 1H), 9.11 (m, 2H), 8.57 (s, |
| | 1-yl]methyl}phenyl)-4- | | | 1H), 8.44 (br s, 1H), 7.86 (d, 2H), |
| | [(3S)-piperidin-3- | | | 7.77 (d, 2H), 7.66 (br s, 1H), 4.66 (m, 1H), |
| | ylamino]thieno[3,2- | | | 4.41 (m, 2H), 3.80-2.93 (m, 16H), |
| | c]pyridine-7- | | | 3.27 (s, 3H), 2.02 (m, 2H), 1.82 (m, 2H) |
| | carboxamide | | | |
| 104 | 2-{4- | 395.53 | 396 | 9.25 (br s, 1H), 9.01 (m, 2H), 8.89 (m, |
| | [(methylamino)methyl]phenyl}- | | | 1H), 8.56 (s, 1H), 8.54 (br s, 1H), |
| | 4-[(3S)- | | | 8.09 (br s, 1H), 7.81 (d, 2H), 7.61 (d, 2H), |
| | piperidin-3- | | | 7.43 (br s, 1H), 4.57 (m, 1H), 4.15 (m, |
| | ylamino]thieno[3,2- | | | 2H), 3.18 (m, 2H), 2.98 (m, 2H), 2.57 (t, |
| | c]pyridine-7- | | | 1H), 2.00 (m, 2H), 1.74 (m, 2H) |
| | carboxamide | | | |
| 105 | 2-[4-(piperazin-1- | 450.61 | 451 | 8.91 (br, 2H), 8.55 (s, 1H), 8.45-7.10 (m, |
| | ylmethyl)phenyl]-4- | | | 8H), 4.57 (m, 1H), 3.57 (m, 2H), |
| | [(3S)-piperidin-3- | | | 3.44-2.56 (m, 12H), 1.99 (m, 2H), 1.74 (m, |
| | ylamino]thieno[3,2- | | | 2H) |
| | c]pyridine-7- | | | |
| | carboxamide | | | |
| 106 | 2-(4- | 437.57 | 438 | 9.67 (br s, 1H), 9.41 (br s, 1H), 9.00 (br |
| | {[acetyl(methyl)amino]methyl}phenyl)- | | | s, 1H), 8.92 (br s, 1H), 8.52 (s, 1H), |
| | 4- | | | 8.47 (br s, 1H), 7.82-7.63 (m, 3H), 7.35 (d, |
| | [(3S)-piperidin-3- | | | 2H), 4.69-4.48 (m, 3H), 3.47 (m, 1H), |
| | ylamino]thieno[3,2- | | | 3.26 (m, 1H), 3.17 (m, 1H), 3.02 (m, |
| | c]pyridine-7- | | | 1H), 2.97-2.77 (m, 3H), 2.10-1.93 (m, |
| | carboxamide | | | 5H), 1.81 (m, 2H) |
| 107 | 2-{3-[(4- | 492.65 | 493 | 10.98 (br s, 1H), 9.46 (br s, 1H), 8.97 (br |
| | acetylpiperazin-1- | | | s, 1H), 8.76 (br s, 1H), 8.56 (s, 1H), |
| | yl)methyl]phenyl}-4- | | | 8.27 (br s, 1H), 7.94 (s, 1H), 7.89 (m, 1H), |
| | [(3S)-piperidin-3- | | | 7.61 (m, 3H), 4.59 (m, 1H), 4.41 (m, |
| | ylamino]thieno[3,2- | | | 2H), 4.10-2.80 (m, 12H), 2.10-1.95 (m, |
| | c]pyridine-7- | | | 5H), 1.78 (m, 2H) |
| | carboxamide | | | |
| 108 | 2-(3- | 437.57 | 438 | 9.38 (br s, 1H), 8.90 (m, 2H), 8.60 (br s, |
| | {[acetyl(methyl)amino]methyl}phenyl)- | | | 1H), 8.54 (m, 1H), 8.26 (m, 1H), |
| | 4- | | | 7.73-7.43 (m, 4H), 7.24 (m, 1H), 4.64 (m, |
| | [(3S)-piperidin-3- | | | 1H), 4.56 (m, 2H), 3.46 (m, 1H), |
| | ylamino]thieno[3,2- | | | 3.23-2.80 (m, 6H), 2.12-1.93 (m, 5H), |
| | c]pyridine-7- | | | 1.77 (m, 2H) |
| | carboxamide | | | |
| 109 | 2-(3- | 473.62 | 474 | 9.34 (br s, 1H), 8.94 (br s, 1H), 8.55 (m, |
| | {[methyl(methylsulfonyl)amino]methyl}phenyl)- | | | 2H), 8.22 (br s, 1H), 7.71 (m, 2H), |
| | 4-[(3S)-piperidin-3- | | | 7.52 (m, 2H), 7.37 (m, 1H), 4.56 (m, 1H), |
| | ylamino]thieno[3,2- | | | 4.31 (m, 2H), 3.46 (m, 1H), 3.19 (m, |
| | c]pyridine-7- | | | 1H), 3.13-2.90 (m, 5H), 2.71-2.53 (m, |
| | carboxamide | | | 3H), 2.01 (m, 2H), 1.77 (m, 2H) |
| 110 | 2-{4-[(4- | 492.65 | 493 | 11.37 (br s, 1H), 9.63 (br s, 1H), |
| | acetylpiperazin-1- | | | 9.02 (m, 1H), 8.56 (s, 1H), 8.39 (br s, 1H), |
| | yl)methyl]phenyl}-4- | | | 7.86 (d, 2H), 7.72 (d, 2H), 7.64 (br s, |
| | [(3S)-piperidin-3- | | | 1H), 4.63 (m, 1H), 4.40 (m, 2H), |
| | ylamino]thieno[3,2- | | | 4.03-2.86 (m, 12H), 2.09-1.95 (m, 2H), |
| | c]pyridine-7- | | | 2.02 (s, 3H), 1.81 (m, 2H) |
| | carboxamide | | | |
| 111 | 2-(4- | 473.62 | 474 | 9.50 (br, 1H), 8.98 (br, 1H), 8.71 (br, |
| | {[methyl(methylsulfonyl)amino]methyl}phenyl)- | | | 1H), 8.54 (s, 1H), 8.28 (br, 1H), 7.79 (d, |
| | 4-[(3S)-piperidin-3- | | | 2H), 7.59 (br s, 1H), 7.46 (d, 2H), |
| | ylamino]thieno[3,2- | | | 4.59 (br, 1H), 4.28 (s, 2H), 3.48 (m, 1H), |
| | c]pyridine-7- | | | 3.16 (m, 2H), 2.98 (m, 1H), 2.97 (s, 3H), |
| | carboxamide | | | 2.69 (s, 3H), 2.10-1.93 (m, 2H), 1.85-1.71 (m, |
| | | | | 2H) |

### Example 112

### 2-(4-{[(1-methyl-1H-indol-2-yl)carbonyl]amino}phenyl)-4-[(3S)-piperidin-3-ylamino]thieno[3,2-c]pyridine-7-carboxamide

1-methyl-N-[4-(4,4,5,5-tetramethyl-1,3,2-dioxaborolan-2-yl)phenyl]-1H-indole-2-carboxamide. To a solution of 1-methyl-1H-indole-2-carboxylic acid (364 mg, 2.08 mmol) in CH₂Cl₂ (5.00 mL) at 0° C. is added drop wise oxalyl chloride (0.536 mL, 6.24 mmol). To this solution is added two drops of DMF and the resulting solution is stirred at reflux overnight whereupon the black solution is then concentrated in vacuo. The resulting residue is diluted with 5 mL of CH₂Cl₂ and added drop wise to a solution of [4-(4,4,5,5-tetramethyl-1,3,2-dioxaborolan-2-yl)phenyl]amine (0.50 g, 2.28 mmol) in 5 mL of CH₂Cl₂ and N,N-diisopropylethylamine (DIPEA, 0.56 mL, 3.12 mmol) at 0° C. The resulting mixture is stirred at rt for three hours. The resulting mixture is then extracted with water (100 mL) and washed with saturated sodium chloride. The organic layer is dried over MgSO₄, filtered, and concentrated under reduced pressure to afford, after column chromatography (0-100% EtOAc/hexanes), the title compound (0.417 mg, 53% yield). ¹H NMR δ 10.45 (s, 1H), 7.82 (m, 2H), 7.67 (m, 2H), 7.58 (d, 1H), 7.56 (m, 2H), 7.36 (m, 2H), 4.01 (s, 3H), 1.29 (s, 12H). LCMS (ES, M+H=377).

tert-butyl (3S)-3-{[7-cyano-2-(4-{[1(1-methyl-1H-indol-2-yl)carbonyl]amino}phenyl)thieno[3,2-c]pyridin-4-yl]amino}piperidine-1-carboxylate. To tert-butyl (3S)-3-[(2-bromo-7-cyanothieno[3,2-c]pyridin-4-yl)amino]piperidine-1-carboxylate (203 mg, 0.464 mmol) is added cesium carbonate (452 mg, 1.39 mmol), 1-methyl-N-[4-(4,4,5,5-tetramethyl-1,3,2-dioxaborolan-2-yl)phenyl]-1H-indole-2-carboxamide (262 mg, 0.696 mmol), Pd(PPh₃)₄ (53.6 mg, 0.0464 mmol), and dioxane/water (2 mL/1 mL). The reaction is heated to 80° C. for one hour whereupon the reaction is cooled to rt, filtered, and purified using silica gel chromatography (0-100% EtOAc/hexanes) to afford the title compound (156 mg, 56% yield). LCMS (ES, M+H=607).

2-(4-{[(1-methyl-1H-indol-2-yl)carbonyl]amino}phenyl)-4-[(3S)-piperidin-3-ylamino]thieno[3,2-c]pyridine-7-carboxamide. To a flask containing tert-butyl (3S)-3-{[7-cyano-2-(4-{[(1-methyl-1H-indol-2-yl)carbonyl]amino}phenyl)thieno[3,2-c]pyridin-4-yl]amino}piperidine-1-carboxylate is added 5.00 mL of 12 N HCl. The reaction mixture is stirred at rt and monitored by LCMS. Additional 12 N HCl is added every twelve hours to afford complete conversion to the desired product. Upon completion, the reaction mixture is diluted with water and concentrated under reduced pressure to yield product, which is purified by silica gel chromatography (CH₂Cl₂ to 20% MeOH/CH₂Cl₂/3% NH₄OH) to afford the title compound. ¹H NMR δ 10.45 (s, 1H), 8.50 (s, 1H), 8.14 (s, 1H), 7.94 (m, 2H), 7.73 (m, 3H), 7.59 (d, 1H), 7.35 (m, 2H), 7.15 (m, 2H), 4.19 (m, 1H), 4.03 (s, 3H), 3.14 (m, 2H), 2.87 (m, 2H), 1.98 (m, 1H), 1.74 (m, 1H), 1.53 (m, 2H). LCMS (ES, M+H=525).

The following examples 113-115 are prepared in a similar fashion.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 113 | 2-(3-{[(1-methyl-1H-indol-2- | 524.65 | 525 | 10.44 (s, 1H), 8.53 (s, 1H), 8.32 (s, 1H), |
| | yl)carbonyl]amino}phenyl)- | | | 8.20 (s, 1H), 7.73 (m, 3H), 7.59 (d, 2H), |
| | 4-[(3S)-piperidin-3- | | | 7.49 (s, 1H), 7.39 (s, 1H), 7.32 (m, 2H), |
| | ylamino]thieno[3,2- | | | 7.15 (m, 1H), 4.22 (m, 1H), 4.05 (s, 3H), |
| | c]pyridine-7-carboxamide | | | 3.19 (m, 2H), 2.90 (m, 2H), 1.99 (m, |
| | | | | 1H), 1.71 (m, 1H), 1.54 (m, 2H) |
| 114 | 2-(4-fluoro-3-{[(1-methyl- | 542.64 | 543 | 10.27 (s, 1H), 9.23 (br s, 1H), |
| | 1H-indol-3- | | | 8.90 (br s, 1H), 8.56 (s, 1H), 8.40 (m, |
| | yl)carbonyl]amino}phenyl)- | | | 1H), 8.10 (m, 1H), 7.72 (d, 1H), |
| | 4-[(3S)-piperidin-3- | | | 7.62 (m, 1H), 7.46 (m, 2H), 7.34 (m, 2H), |
| | ylamino]thieno[3,2- | | | 7.16 (m, 2H), 6.99 (m, 1H), 4.55 (m, |
| | c]pyridine-7-carboxamide | | | 1H), 4.04 (s, 3H), 3.48 (m, 1H), |
| | | | | 3.19 (m, 1H), 2.95 (m, 2H), 2.01 (m, 2H), |
| | | | | 1.74 (m, 2H) |
| 115 | 2-(3-fluoro-4-{[(1-methyl- | 542.64 | 543 | 10.2 (s, 1H), 8.81 (m, 1H), 8.57 (m, |
| | 1H-indol-2- | | | 1H), 8.33 (m, 1H), 8.04 (m, 1H), |
| | yl)carbonyl]amino}phenyl)- | | | 7.81 (t, 1H), 7.65 (m, 6H), 7.34 (m, |
| | 4-[(3S)-piperidin-3- | | | 1H), 7.14 (m, 1H), 6.95 (m, 1H), |
| | ylamino]thieno[3,2- | | | 4.53 (m, 1H), 4.03 (s, 3H), 3.49 (m, |
| | c]pyridine-7-carboxamide | | | 2H), 3.24 (m, 1H), 2.95 (m, 1H), |
| | | | | 2.02 (m, 2H), 1.69 (m, 2H) |

### Example 116

### 2-bromo-4-[(3S)-piperidin-3-ylamino]thieno[3,2-c]pyridine-7-carboxamide

To solid tert-butyl (3S)-3-[(2-bromo-7-cyanothieno[3,2-c]pyridine-4-yl)amino]piperidine-1-carboxylate (70 mg, 0.20 mmol) is added 4 mL of conc. HCl, and the solution is stirred at rt for 2 days. The title compound (78 mg, 100%) is obtained as the hydrochloride salt after removal of the solvent in vacuo and drying under high vacuum. ¹H NMR δ 1.50 (m, 2H), 1.67 (m, 1H), 1.93 (m, 1H), 2.81 (m, 2H), 3.11 (m, 1H), 3.24 (m, 1H), 4.11 (m, 1H), 7.20 (m, 1H), 7.34 (m, 1H), 7.95 (br s, 1H), 8.52 (s, 1H). LCMS (ES, M+H=356).

### Example 117

### 2-phenyl-4-[(3S)-piperidin-3-yloxy]thieno[3,2-c]pyridine-7-carboxamide

tert-butyl (3S)-3-[(2-bromo-7-cyanothieno[3,2-c]pyridin-4-yl)oxy]piperidine-1-carboxylate. To a stirred solution of tert-butyl (3S)-3-hydroxypiperidine-1-carboxylate (1.4 g, 7 mmol) in THF (10 mL) is slowly added NaH (0.3 g, 7 mmol; 60% in mineral oil) portion wise. After 15 minutes, 2-bromo-4-chlorothieno[3,2-c]pyridine-7-carbonitrile (1.4 g, 5.1 mmol) suspended in THF (10 mL) is added slowly to a preformed solution of alkoxide. The reaction mixture is stirred at rt for 1 h and then diluted with 80 mL of water. Extraction into EtOAc, followed by washing with sat. NaHCO₃, sat. NaCl, and drying over Na₂SO₄ gives the product as a brown solid (˜2 g, 91%), which is used directly in the next step. ¹H NMR δ 8.66 (s, 1H), 7.79 (s, 1H), 5.26 (m, 1H), 4.13 (m, 1H), 3.84 (m, 1H), 2.93 (m, 1H), 1.94 (m, 3H), 1.50 (m, 2H), 0.92 (s, 9H). LCMS (ES, M+H=438, 440).

tert-butyl (3S)-3-[(7-cyano-2-phenylthieno[3,2-c]pyridin-4-yl)oxy]piperidine-1-carboxylate. A mixture of tert-butyl (3S)-3-[(2-bromo-7-cyanothieno[3,2-c]pyridin-4-yl)oxy]piperidine-1-carboxylate (2 g. 4.5 mmol), phenylboronic acid (0.83 g, 6.8 mmol), Pd(PPh₃)₄ (0.8 g, 0.68 mmol), and cesium carbonate (4.4 g, 13.6 mmol), are dissolved in water (5 mL), and dioxane (20 mL). This reaction mixture is stirred at 80° C. for 1 h under a nitrogen atmosphere, and then allowed to cool to rt. The water is removed with a pipette and the dioxane is removed under vacuum. The residue is purified by MPLC (SiO₂; 20-50% EtOAc/Hexanes) gave the title compound (1.0 g, 45%; 2 steps). ¹H NMR δ 8.62 (s, 1H) 7.85 (d, 2H) 7.42-7.54 (m, 3H) 6.74 (d, 1H) 5.29 (s, 1H) 4.15 (m, 1H) 3.83 (m, 1H) 3.35 (m, 1H) 2.97 (m, 1H) 1.98 (s, 3H) 1.52 (s, 1H) 0.90 (s, 9H). LCMS (ES, M+H=436).

2-phenyl-4-[(3S)-piperidin-3-yloxy]thieno[3,2-c]pyridine-7-carboxamide. A solution of tert-butyl (35)-3-[(7-cyano-2-phenylthieno[3,2-c]pyridin-4-yl)oxy]piperidine-1-carboxylate (1.0 g, 2.3 mmol) and 12N HCl (conc., 15 mL) is stirred for 12 hours. Water (100 mL) is added and the solution is concentrated to dryness in vacuo. The white solid obtained is dissolved in 100 mL of MeOH and concentrated in vacuo to give the title compound (0.86 g, 96%) after drying under high vacuum. ¹H NMR δ 9.56 (d, 1H) 9.10 (s, 1H) 8.67 (s, 1H) 8.32 (s, 1H) 8.28 (s, 1H) 7.87 (d, 2H) 7.66 (s, 1H) 7.49 (t, 2H) 7.40 (t, 1H), 5.64 (s, 1H) 3.41 (s, 2H) 3.17-3.28 (m, 1H) 3.03 (d, 1H) 1.91-2.06 (m, 3H) 1.65-1.79 (m, 1H). LCMS (ES, M+H=354).

Examples 118-128 are made in a similar fashion from the appropriate starting materials.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 118 | 4-[2- | 395.52 | 396 | 8.79 (br s, 2H), 8.67 (s, 1H), 8.30 (br s, |
| | (cyclohexylamino)ethoxy]- | | | 1H), 8.02 (m, 1H), 7.82 (d, 2H), |
| | 2-phenylthieno[3,2- | | | 7.72 (m, 1H), 7.51 (m, 2H), 7.43 (m, 1H), |
| | c]pyridine-7- | | | 4.75 (m, 1H), 3.49 (m, 2H), 3.15 (m, |
| | carboxamide | | | 1H), 2.08 (m, 2H), 1.76 (m, 2H), |
| | | | | 1.59 (m, 1H), 1.26 (m, 5H) |
| 119 | 4-[2- | 327.41 | 328 | 8.48 (s, 1H), 8.28 (m, 1H), 8.21 (m, |
| | (methylamino)ethoxy]- | | | 1H), 7.85 (d, 2H), 7.56 (m, 4H), |
| | 2-phenylthieno[3,2- | | | 7.41 (m, 1H), 3.93 (m, 2H), 3.78 (m, 2H), |
| | c]pyridine-7- | | | 3.50 (s, 3H) |
| | carboxamide | | | |
| 120 | 2-phenyl-4-[(3S)- | 339.42 | 340 | 8.45 (m, 1H), 8.22 (m, 1H), 7.89 (m, |
| | pyrrolidin-3- | | | 2H), 7.69 (m, 1H), 7.46 (m, 3H), |
| | yloxy]thieno[3,2- | | | 4.52 (m, 1H), 4.08 (m, 2H), 3.63 (m, 2H), |
| | c]pyridine-7- | | | 2.08 (m, 2H) |
| | carboxamide | | | |
| 121 | 4-{[(2R)-2- | 327.41 | 328 | 8.66 (s, 1H), 8.30 (m, 2H), 8.18 (s, |
| | aminopropyl]oxy}-2- | | | 1H), 7.84 (m, 2H), 7.72 (m, 1H), |
| | phenylthieno[3,2- | | | 7.48 (m, 4H), 4.66 (m, 1H), 4.45 (m, 1H), |
| | c]pyridine-7- | | | 3.76 (m, 1H), 1.36 (d, 3H) |
| | carboxamide | | | |
| 122 | 4-[2-(isopropylamino)ethoxy]- | 355.46 | 356 | 8.89 (br s, 1H), 8.68 (s, 1H), 8.30 (m, |
| | 2-phenyl- | | | 1H), 8.06 (m, 1H), 7.80 (m, 3H), |
| | thieno[3,2-c]pyridine-7- | | | 7.46 (m, 3H), 4.75 (m, 1H), 3.89 (m, 2H), |
| | carboxamide | | | 3.46 (m, 2H), 1.30-1.22 (d, 6H) |
| 123 | 4-{[(2S)-2- | 327.41 | 328 | 8.66 (s, 1H), 8.23 (m, 3H), 7.83 (d, |
| | aminopropyl]oxy}-2- | | | 2H), 7.71 (m, 1H), 7.49 (m, 4H), |
| | phenylthieno[3,2- | | | 4.66 (m, 1H), 4.46 (m, 1H), 3.75 (m, 1H), |
| | c]pyridine-7- | | | 1.36 (d, 3H) |
| | carboxamide | | | |
| 124 | 4-{[(2S)-2-amino-4- | 369.49 | 370 | 8.67 (s, 1H), 8.30 (m, 2H), 8.21 (s, |
| | methylpentyl]oxy}-2- | | | 1H), 7.84 (m, 2H), 7.71 (m, 1H), |
| | phenylthieno[3,2- | | | 7.52 (m, 2H), 7.43 (m, 2H), 4.70 (m, 1H), |
| | c]pyridine-7- | | | 4.51 (m, 1H), 3.48 (m, 1H), 1.80 (m, |
| | carboxamide | | | 1H), 1.62 (m, 2H), 0.94 (m, 6H) |
| 125 | 4-(2-amino-3- | 343.41 | 344 | 8.67 (s, 1H), 8.35 (m, 3H), 8.18 (s, |
| | hydroxypropoxy)-2- | | | 1H), 7.84 (m, 3H), 7.71 (s, 1H), |
| | phenylthieno[3,2- | | | 7.48 (m, 3H), 4.64 (m, 2H), 3.78 (m, 2H), |
| | c]pyridine-7- | | | 3.08 (m, 1H) |
| | carboxamide | | | |
| 126 | 4-[(3S)-piperidin-3- | 354.43 | 355 | 9.57 (m, 2H), 9.44 (s, 1H), 9.02 (s, |
| | yloxy]-2-pyridin-3- | | | 1H), 8.87 (d, 1H), 8.81 (d, 1H), |
| | ylthieno[3,2-c]pyridine- | | | 8.78 (s, 1H), 8.41 (br s, 1H), 8.00 (dd, 1H), |
| | 7-carboxamide | | | 7.78 (br s, 1H), 5.67 (m, 1H), 3.43 (s, |
| | | | | 2H), 3.22 (m, 1H), 3.05 (m, 1H), |
| | | | | 1.99 (m, 3H), 1.73 (m, 1H) |
| 127 | 4-[(3R)-piperidin-3- | 354.43 | 355 | 9.35 (m, 1H), 9.27 (s, 1H), 9.12 (m, |
| | yloxy]-2-pyridin-3- | | | 1H), 8.73 (s, 1H), 8.72 (m, 1H), |
| | ylthieno[3,2-c]pyridine- | | | 8.67 (s, 1H), 8.56 (d, 1H), 8.36 (m, 1H), |
| | 7-carboxamide | | | 7.78 (m, 2H), 5.67 (m, 1H), 3.43 (s, |
| | | | | 2H), 3.22 (m, 1H), 3.05 (m, 1H), |
| | | | | 1.99 (m, 3H), 1.73 (m, 1H) |
| 128 | 4-{[(3S)-1-methyl- | 367.47 | 368 | 8.63 (s, 1H), 8.20 (br s, 1H), 7.85 (d, |
| | piperidin-3-yl]oxy}-2- | | | 2H), 7.70 (s, 1H), 7.63 (s, 1H), |
| | phenylthieno[3,2- | | | 7.48 (m, 2H), 7.40 (m, 1H), 5.33 (m, 1H), |
| | c]pyridine-7- | | | 3.16 (m, 2H), 2.95 (m, 2H), 2.20 (s, |
| | carboxamide | | | 3H), 2.08 (m, 2H), 1.83 (m, 2H) |

### Example 129

### 2-phenyl-4-(piperidin-3-ylthio)thieno[3,2-c]pyridine-7-carboxamide

Prepared in a similar fashion to Example 17 but using benzyl 3-mercaptopiperidine-1-carboxylate (synthesis described below) as the starting material in the first step. ¹H NMR δ 9.23 (br, 2H), 8.91 (s, 1H), 8.45 (s, 1H), 7.88 (d, 2H), 7.83 (br, 1H), 7.82 (s, 1H), 7.50 (t, 2H), 7.43 (t, 1H), 4.41 (m, 1H), 3.63 (m, 1H), 3.23 (m, 1H), 3.09 (m, 1H), 2.94 (m, 1H), 2.22-2.13 (m, 1H), 1.96-1.72 (m, 3H). LCMS (ES, M+H=370).

benzyl 3-(acetylthio)piperidine-1-carboxylate. To triphenylphosphine (3.40 g, 13.0 mmol) and diisopropyl azodicarboxylate (2.65 mL, 13.7 mmol) in THF (10 mL) at 0° C. is added benzyl 3-hydroxypiperidine-1-carboxylate (2.57 g, 10.9 mmol), followed by thiolacetic acid (1.00 mL, 14.0 mmol). The reaction mixture is heated to 70° C. for 18 h. After cooling, the reaction mixture is concentrated in vacuo and purified by MPLC (gradient: 5 to 20% EtOAc/hexanes) to yield the desired product as a yellow oil (1.02 g, 3.49 mmol, 32%). ¹H NMR δ 7.41-7.27 (m, 5H), 5.12-5.01 (m, 2H), 3.75 (m, 1H), 3.58-3.13 (m, 4H), 2.28 (br s, 3H), 1.91 (m, 1H), 1.65-1.43 (m, 3H). LCMS (ES, M+Na=316).

benzyl 3-mercaptopiperidine-1-carboxylate. To benzyl 3-(acetylthio)piperidine-1-carboxylate (541 mg, 1.85 mmol), in MeOH (20 mL) is added NaSMe (582 mg, 8.31 mmol) in MeOH (10 mL). The mixture is stirred for 2 h at which point LCMS analysis indicated complete consumption of starting material. The reaction mixture is concentrated in vacuo and the residue partitioned between EtOAc and 0.5 M HCl. The organic layer is concentrated to yield the free thiol as a yellow oil. (463 mg, 1.85 mmol, >98%). ¹H NMR δ 7.41-7.26 (m, 5H), 5.13-5.00 (m, 2H), 3.98 (m, 1H), 3.76 (m, 1H), 2.90 (m, 1H), 2.80 (m, 1H), 2.70 (d, 1H), 1.99 (m, 1H), 1.65 (m, 1H), 1.40 (m, 2H). LCMS (ES, M+Na=274).

### Example 130

### 4-{[(3S)-1-methylpiperidin-3-yl]amino}-2-phenylthieno[3,2-e]pyridine-7-carboxamide

(3S)-1-methylpiperidin-3-amine. To a solution of tert-butyl (3S)-3-aminopiperidine-1-carboxylate (8.89 g, 44.4 mmol) in THF (176 mL) at 0° C. is added drop wise 1M lithium aluminum hydride in THF (88.0 mL, 88.8 mmol). The resulting grey solution is warmed to rt and stirred under nitrogen overnight. A solution of 10% Rochelle's salt is added to the mixture at 0° C. until the bubbling ceased. The resulting mixture is extracted with copious amounts of EtOAc, followed by 1/1 MeOH/CH₂Cl₂. The combined organic layers are dried over MgSO₄, filtered, and concentrated in vacuo to afford the title compound, which is used directly in the next reaction. GCMS (m/z 114).

2-bromo-4-{[(3S)-1-methylpiperidin-3-yl]amino}thieno[3,2-c]pyridine-7-carbonitrile. To a solution of 2-bromo-4-chlorothieno[3,2-c]pyridine-7-carbonitrile (400 mg, 1.46 mmol) in NMP (2.0 mL) is added potassium carbonate (605 mg, 4.38 mmol) and (38)-1-methylpiperidin-3-amine (333 mg, 2.92 mmol). The reaction mixture is heated to 130° C. until LCMS indicated the completion of the reaction. The reaction mixture is cooled to rt and approximately 100 mL of water is added. The resulting solid is filtered and vacuum dried to afford the title compound. LCMS (ES, M+H=353).

4-{[(3S)-1-methylpiperidin-3-yl]amino}-2-phenylthieno[3,2-c]pyridine-7-carbonitrile. To 2-bromo-4-{[(3S)-1-methylpiperidin-3-yl]amino}thieno[3,2-c]pyridine-7-carbonitrile (512 mg, 1.46 mmol) is added cesium carbonate (1.43 g, 4.38 mmol), phenyl boronic acid (306 mg, 2.19 mmol), Pd(PPh₃)₄ (169 mg, 0.146 mmol), and dioxane/water (4 mL/2 mL). The reaction is heated to 80° C. for one hour whereupon the reaction is cooled to rt, filtered, and purified using silica gel chromatography (100% hexanes to 100% EtOAc) to afford the title compound. LCMS (ES, M+H=349).

4-{[(3S)-1-methylpiperidin-3-yl]amino}-2-phenylthieno[3,2-c]pyridine-7-carboxamide. To a flask containing 4-{[(3S)-1-methylpiperidin-3-yl]amino}-2-phenylthieno[3,2-c]pyridine-7-carbonitrile is added 5.00 mL of 12 N HCl. The reaction mixture is stirred at rt and monitored by LCMS. Additional 12 N HCl is added after twelve hours to afford complete conversion to the desired product. Upon completion, the reaction mixture is diluted with water and concentrated under reduced pressure to yield product, which is purified by silica gel chromatography (100% CH₂Cl₂ to 20% MeOH/CH₂Cl₂/3% NH₄OH) to afford the title compound. ¹H NMR δ 8.52 (s, 1H), 8.20 (s, 1H), 7.74-7.71 (m, 3H), 7.48-7.36 (m, 4H), 7.21 (d, 1H), 4.28 (m, 1H), 2.96 (m, 2H), 2.69 (m, 2H), 1.93-1.55 (m, 4H). LCMS (ES, M+H=367).

### Example 131

### 2-(4-cyanophenyl)-4-[(3S)-piperidin-3-ylamino]thieno[3,2-c]pyridine-7-carboxamide

Solid 2-bromo-4-[(3S)-piperidin-3-ylamino]thieno[3,2-c]pyridine-7-carboxamide (75 mg, 0.2 mmol), 4-cyanophenylboronic acid (44.1 mg, 0.30 mmol), cesium carbonate (260 mg, 0.80 mmol), Pd(PPh₃)₄ (23 mg, 0.02 mmol), is dissolved in 2 ml of dioxane and 0.5 ml of water. The reaction mixture is heated to 80° C. for 2 hours. The solvent is removed in vacuo and the residue purified by preparatory HPLC(H₂O/CH₃CN/0.1% TFA gradient). The trifluoroacetate salt obtained after lyophilization is dissolved in MeOH, and treated with 4N HCl in dioxane, then stirred at rt for several hours. The title compound is isolated as the hydrochloride salt (44 mg, 54%) after removal of the solvent in vacuo and drying under high vacuum. ¹H NMR δ 9.36 (s, 1H), 8.97 (s, 1H), 8.83 (s, 1H), 8.60 (s, 1H), 8.22 (s, 1H), 7.96 (q, 4H), 7.56 (s, 1H), 4.60 (m, 1H), 3.21 (m, 2H), 3.02 (m, 2H), 2.05 (m, 2H), 1.77 (m, 2H). LCMS (ES, M+H=378).

The following example 132 is prepared in a similar fashion.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 132 | (4-{7-(aminocarbonyl)- | 410.5 | 411 | 8.74 (s, 2H), 8.55 (s, 1H), 8.15 (s, 1H), |
| | 4-[(3S)-piperidin-3- | | | 8.05 (s, 1H), 7.69 (d, 2H), 7.58 (s, 1H), |
| | ylamino]thieno[3,2- | | | 7.39 (d, 2H), 4.50 (s, 1H), 3.24 (m, 2H), |
| | c]pyridin-2- | | | 2.89 (m, 2H), 2.08 (m, 2H), 1.76 (m, |
| | yl}phenyl)acetic acid | | | 2H) |

### Example 133

### 4-{[(2S)-2-amino-3-hydroxypropyl]amino}-2-phenylthieno[3,2-c]pyridine-7-carboxamide

methyl 3-amino-N-[(benzyloxy)carbonyl]-L-alaninate. To a flask containing methyl 3-amino-N-[(benzyloxy)carbonyl]-L-alaninate (5.0 g, 21.0 mmol) equipped with a magnetic stir bar is added dry MeOH (100 mL). HCl gas is bubbled into the solution/slurry for about 10 minutes with stirring. The exothermic reaction goes from cloudy white to clear/colorless after about 2 minutes. This solution is stirred overnight before concentrating in vacuo and drying to give the title compound (6.0 g, 98%) as a white crystalline hydrochloride salt. ¹H NMR δ 3.05 (t, 1H), 3.19 (t, 1H), 3.70 (s, 3H), 4.45 (m, 1H), 5.10 (s, 2H), 7.38 (m, 5H), 7.95 (d, 1H), 8.35 (br s, 3H). LCMS (ES, M+H=253).

methyl N-[(benzyloxy)carbonyl]-3-[(2-bromo-7-cyanothieno[3,2-c]pyridin-4-yl)amino]-L-alaninate. To a stirred solution of 4-chloro-2-bromo-thieno[3,2-c]pyridine-7-carbonitrile (0.53 g, 1.9 mmol) and methyl 3-amino-N-[(benzyloxy)carbonyl]-L-alaninate hydrochloride salt (0.66 g, 2.3 mmol) in NMP (4 mL) is added potassium carbonate (0.44 g, 3.2 mmol). The heterogeneous mixture is heated to 80° C. for 2 h, cooled to rt, and then added to 50 mL of water. The product (1.2 g) is isolated by filtration and is dried to yield a dark brown solid. The solid is purified using MPLC (SiO₂; 0-100% EtOAc/Hexanes) to give the title compound (0.41 g). LCMS (ES, M+H=489, 491; M−H=487, 489).

methyl N-[(benzyloxy)carbonyl]-3-[(7-cyano-2-phenylthieno[3,2-c]pyridin-4-yl)amino]-L-alaninate. To a nitrogen purged flask containing methyl N-[(benzyloxy)carbonyl]-3-[(2-bromo-7-cyanothieno[3,2-c]pyridin-4-yl)amino]-L-alaninate (0.41 g. 0.85 mmol) is added phenylboronic acid (0.21 g, 1.7 mmol), Pd(PPh₃)₄ (0.10 g, 0.085 mmol), cesium carbonate (0.83 g, 2.5 mmol), water (2 mL), and dioxane (6 mL). This reaction mixture is brought to 80° C. for 15 minutes, and then allowed to cool to rt. Purification by MPLC (SiO₂; 0-50% EtOAc/Hexanes) gives the title compound (89 mg). LCMS (ES, M+H=487).

benzyl [(1S)-2-[(7-cyano-2-phenylthieno[3,2-c]pyridin-4-yl)amino]-1-(hydroxymethyl)ethyl]carbamate. To a flask containing methyl N-[(benzyloxy)carbonyl]-3-[(7-cyano-2-phenylthieno[3,2-c]pyridin-4-yl)amino]-L-alaninate (89 mg, 0.18 mmol) dissolved in THF (9.5 mL)/MeOH (0.5 mL), is added NaBH₄ (0.014 g, 0.37 mmol) under a nitrogen atmosphere. The reaction is stirred at rt and monitored by LCMS. After 40 minutes, the reaction is complete. The solvent is concentrated in vacuo to give the title compound, 83 mg (100%), which is used directly in the next. LCMS (ES, M+H=459).

4-{[(2S)-2-amino-3-hydroxypropyl]amino}-2-phenylthieno[3,2-c]pyridine-7-carboxamide. In a flask equipped with a magnetic stir bar is added benzyl [(1S)-2-[(7-cyano-2-phenylthieno[3,2-c]pyridin-4-yl)amino]-1-(hydroxymethyl)ethyl]carbamate (83 mg, 0.18 mmol) and 12N HCl (5 mL). This slurry is stirred for 24 hours. The solvent is removed in vacuo, and the residue is purified by MPLC (SiO₂; 50-100% NH₄OH/MeOH/CH₂Cl₂ (1:7:92)) to give the title compound (23 mg). ¹H NMR δ 1.35 (s, 2H), 2.75 (m, 1H), 3.16 (m, 2H), 3.33 (m, 1H), 3.87 (q, 1H), 4.45 (t, 1H), 7.04 (s, 1H), 7.14 (t, 1H), 7.28 (m, 3H), 7.50 (d, 2H), 7.68 (s, 1H), 7.94 (s, 1H), 8.30 (s, 1H). LCMS (ES, M+H=343).

The following example 134 is prepared in an analogous fashion to Example 133 using methyl 3-amino-N-[(benzyloxy)carbonyl]-D-alaninate instead of methyl 3-amino-N-[(benzyloxy)carbonyl]-L-alaninate in the first step.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 134 | 4-{[(2R)-2-amino-3- | 342.42 | 343 | 8.57 (s, 1H), 8.21 (s, 1H), 7.97 (br s, |
| | hydroxypropyl]amino}- | | | 1H), 7.77 (d, 2H), 7.61 (t, 1H), 7.54 (t, |
| | 2-phenylthieno[3,2- | | | 2H), 7.42 (t, 1H), 7.34 (s, 1H), 4.76 (t, |
| | c]pyridine-7- | | | 1H), 3.62 (m, 1H), 3.46 (m, 2H), |
| | carboxamide | | | 3.30 (m, 1H), 3.05 (t, 1H), 2.0 (br s, 2H) |

### Example 135

### 3-{[7-(aminocarbonyl)-2-phenylthieno[3,2-c]pyridin-4-yl]amino}-D-alanine

N-[(benzyloxy)carbonyl]-3-[(7-cyano-2-phenylthieno[3,2-c]pyridin-4-yl)amino]-D-alanine (54 mg, 0.11 mmol) [an intermediate prepared for Example 134 in an analogous fashion to that prepared in Example 133] is dissolved in 4 mL 12N HCl and stirred overnight. The reaction mixture is evaporated and dried under high vacuum. The title product is isolated as the trifluoroacetate salt (19 mg, 48%) after purification by preparatory HPLC. ¹H NMR δ 8.65 (m, 2H), 8.40 (m, 1H), 8.1-8.3 (br s, 1H), 7.82 (m, 2H), 7.59 (m, 2H), 7.49 (m, 1H), 4.45 (m, 1H), 4.17 (m, 2H), 4.08 (m, 2H), 3.90 (br s, 2H). LCMS (ES, M+H=357).

### Example 136

### 4-[(3S)-piperidin-3-ylamino]-2-pyridin-4-ylthieno[3,2-c]pyridine-7-carboxamide

tert-butyl 3-[(7-cyano-2-pyridin-4-ylthieno[3,2-c]pyridin-4-yl)amino]piperidine-1-carboxylate. To tert-butyl 3-[(2-bromo-7-cyanothieno[3,2-c]pyridin-4-yl)amino]piperidine-1-carboxylate (183 mg, 0.418 mmol) in N,N-dimethylformamide (2.00 mL) is added Pd(PPh₃)₄ (19.3 mg, 0.017 mmol), copper iodide (15.9 mg, 0.084 mmol), and 4-(tributylstannyl)pyridine (185 mg, 0.502 mmol). The reaction mixture is stirred at 80° C. under a nitrogen atmosphere until LCMS indicated completion of the reaction. The resulting black reaction mixture is filtered, rinsed with EtOAc, concentrated under reduced pressure, and purified by silica gel chromatography (100% CH₂Cl₂ to 20% MeOH/CH₂Cl₂) to afford 103 mg (51% yield) of the title compound. LCMS (ES, M+H=436).

4-[(3S)-piperidin-3-ylamino]-2-pyridin-4-ylthieno[3,2-c]pyridine-7-carboxamide. To a flask containing tert-butyl 3-[(7-cyano-2-pyridin-4-ylthieno[3,2-c]pyridin-4-yl)amino]piperidine-1-carboxylate is added 2.00 mL of 12 N HCl. The reaction mixture is stirred at rt and monitored by LCMS. Additional 12 N HCl is added every twelve hours to afford complete conversion to the desired product. Upon completion, the reaction mixture is diluted with water and concentrated under reduced pressure to yield product, which is purified by silica gel chromatography (100% CH₂Cl₂ to 20% MeOH/CH₂Cl₂/3% NH₄OH) to afford the title compound. ¹H NMR δ 1.70-1.56 (m, 2H), 1.99-1.78 (m, 2H), 2.98-2.89 (2H), 3.09 (m, 1H), 3.24 (m, 1H), 4.22 (m, 1H), 7.38 (m, 1H), 7.66 (d, 2H), 7.97 (m, 1H), 8.47 (s, 1H), 8.57 (s, 1H), 8.64 (d, 2H). LCMS (ES, M+H=354).

The following example 137 is prepared in an analogous fashion using the appropriate starting materials.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 137 | 4-[(3S)-piperidin-3- | 353.45 | 354 | 1.54 (m, 2H), 1.70 (m, 1H), 1.99 (m, |
| | ylamino]-2-pyridin-3- | | | 1H), 2.75 (m, 2H), 3.15 (m, 1H), |
| | ylthieno[3,2-c]pyridine- | | | 3.24 (m, 1H), 4.17 (m, 1H), 7.24 (m, 1H), |
| | 7-carboxamide | | | 7.52 (m, 1H), 7.93 (m, 1H), 8.09 (m, |
| | | | | 1H), 8.30 (s, 1H), 8.54 (br s, 2H), |
| | | | | 8.95 (s, 1H) |

### Example 138

### 2-(phenylethynyl)-4-[(3S)-piperidin-3-ylamino]thieno[3,2-c]pyridine-7-carboxamide

2-iodo-4-oxo-4,5-dihydrothieno[3,2-c]pyridine-7-carbonitrile. A solution of 4-oxo-4,5-dihydrothieno[3,2-c]pyridine-7-carbonitrile (4.0 g, 22 mmol) in a 50:50 mixture of DMF/Acetic Acid (32 mL) is charged with N-iodosuccinimide (10.2 g, 44 mmol). The dark reaction mixture is heated to 80° C. for 12 h. After cooling to rt, the reaction is added to ˜150 mL of water while stirring. The pH of the cloudy solution is adjusted to 9-10 with sat. NaHCO₃. The product is obtained by filtration, washing with water, and drying in a vacuum oven (5.0 g, 76%). ¹H NMR δ 12.5 (br s, 1H), 8.31 (d, 1H), 7.83 (s, 1H). LCMS (ES, M+H=303, M−H=301).

4-chloro-2-iodothieno[3,2-c]pyridine-7-carbonitrile. A solution of 2-iodo-4-oxo-4,5-dihydrothieno[3,2-c]pyridine-7-carbonitrile (5.0 g, 16.6 mmol) dissolved in POCl₃ (50 mL) is heated to reflux overnight. After cooling to rt, the reaction is concentrated to dryness under vacuum. The solids are slowly and carefully suspended in ˜300 mL of water. The product is obtained by filtration, followed by washing with water, sat. NaHCO₃, water, and drying in a vacuum oven (4.3 g, 84%). ¹H NMR δ 8.80 (s, 1H), 8.05 (s, 1H). LCMS (ES, M+H=321).

tert-butyl (3S)-3-[(7-cyano-2-iodothieno[3,2-c]pyridin-4-yl)amino]piperidine-1-carboxylate. To a stirred solution of 4-chloro-2-iodothieno[3,2-c]pyridine-7-carbonitrile (2.5 g, 7.8 mmol) and tert-butyl (3S)-3-aminopiperidine-1-carboxylate (1.9 g, 9.4 mmol) in NMP (14 mL) is added potassium carbonate (2.2 g, 15.6 mmol). The heterogeneous mixture is heated to 80° C. for 2 h, cooled to rt, and then added to ˜100-1150 mL of water. Filtration and drying yields the product as a dark brown solid (4.4 g, 100%), which is used directly in the next step without purification. LCMS (ES, M+H=485; M−H, 483).

tert-butyl (3S)-3-{[7-cyano-2-(phenylethynyl)thieno[3,2-c]pyridin-4-yl]amino}piperidine-1-carboxylate. To tert-butyl (3S)-3-[(7-cyano-2-iodothieno[3,2-c]pyridin-4-yl)amino]piperidine-1-carboxylate (150 mg, 310 mmol) in N,N-dimethylformamide (1.00 mL) is added PdCl₂(PPh₃)₂ (16.1 mg, 0.023 mmol), copper iodide (4.40 mg, 0.023 mmol), TEA (0.130 mL, 0.930 mmol), and phenylacetylene (81.7 μL, 0.744 mmol). The reaction mixture is stirred at rt under a nitrogen atmosphere until LCMS indicated completion of the reaction. To the resulting reaction mixture is added 10 mL water followed by extracting the mixture with EtOAc (4×20 mL), drying the organic layers with MgSO₄, filtering, and concentrating the solvent under reduced pressure to afford a black residue, which is purified by preparatory HPLC (5-95% MeCN, H₂O, 0.1% TFA) to afford the title compound. LCMS (ES, M+H=459).

2-(Phenylethynyl)-4-[(3S)-piperidin-3-ylamino]thieno[3,2-c]pyridine-7-carboxamide. To a flask containing tert-butyl (3S)-3-{[7-cyano-2-(phenylethynyl)thieno[3,2-c]pyridin-4-yl]amino}piperidine-1-carboxylate is added 1.00 mL of 12 N HCl. The reaction mixture is stirred at rt and monitored by LCMS. Additional 12 N HCl is added every twelve hours to afford complete conversion to the desired product. Upon completion, the reaction mixture is cooled to 0° C. and treated with 6 N NaOH drop wise until a pH of 12 is obtained. The mixture is extracted with EtOAc in addition to CH₂Cl₂/MeOH (1/1), organic layers are dried over magnesium sulfate, filtered and concentrated in vacuo to yield product which is purified by preparatory HPLC (5-95% MeCN, H₂O, 0.1% TFA) affording the title compound. ¹H NMR δ 1.99-1.63 (m, 4H), 2.91-2.83 (m, 2H), 3.31-3.20 (m, 2H), 4.48 (m, 1H), 7.26 (m, 1H), 7.48 (m, 3H), 7.55 (m, 2H), 8.10 (s, 1H), 8.71-8.57 (overlapping m and s, 2H). LCMS (ES, M+H=377).

The following examples 139-145 are prepared in an analogous fashion to example 1 using tert-butyl (3S)-3-[(7-cyano-2-iodothieno[3,2-c]pyridin-4-yl)amino]piperidine-1-carboxylate in step 8.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 139 | 2-(6-fluoro-5- | 385.47 | 386 | 1.82-1.62 (m, 2H), 2.11-1.94 (m, 2H), |
| | methylpyridin-3-yl)-4- | | | 2.32 (s, 3H), 2.91 (m, 2H), 3.20 (m, |
| | [(3S)-piperidin-3- | | | 1H), 3.45 (m, 1H), 4.50 (m, 1H), |
| | ylamino]thieno[3,2- | | | 7.51 (m, 1H) 8.03 (m, 1H), |
| | c]pyridine-7- | | | 8.17 (overlapping s and m, 2H), 8.40 (s, |
| | carboxamide | | | 1H), 8.57 (s, 1H), 8.69 (s, 1H) |
| 140 | 2-[4- | 395.49 | 396 | 1.78-1.64 (m, 2H), 2.00-1.96 (m, 2H), |
| | (aminocarbonyl)phenyl]- | | | 2.93-2.88 (m, 2H), 3.23 (m, 1H), |
| | 4-[(3S)-piperidin-3- | | | 3.51 (m, 1H), 4.50 (m, 1H), 7.43 (br s, 1H), |
| | ylamino]thieno[3,2- | | | 7.62 (m, 1H), 7.80 (m, 2H), |
| | c]pyridine-7- | | | 8.05-7.98 (m, 4H), 8.30 (s, 1H), 8.56 (s, 1H), |
| | carboxamide | | | 8.76 (br s, 2H) |
| 141 | 2-[3- | 395.49 | 396 | 1.80-1.66 (m, 2H), 2.09-1.94 (m, 2H), |
| | (aminocarbonyl)phenyl]- | | | 2.92 (m, 2H), 3.21 (m, 2H), 4.49 (m, |
| | 4-[(3S)-piperidin-3- | | | 1H), 7.56 (m, 3H), 7.87 (m, 2H), |
| | ylamino]thieno[3,2- | | | 8.17 (s, 1H), 8.28 (s, 1H), 8.36 (br s, 1H), |
| | c]pyridine-7- | | | 8.56 (s, 1H), 8.80 (br m, 2H) |
| | carboxamide | | | |
| 142 | 4-[(3S)-piperidin-3- | 354.44 | 355 | 1.80-1.70 (m, 2H), 2.08-1.91 (m, 2H), |
| | ylamino]-2-pyrimidin-5- | | | 3.15-2.96 (m, 3H), 3.41 (m, 1H), |
| | ylthieno[3,2-c]pyridine- | | | 4.62 (m, 1H), 7.54 (m, 1H), 8.20 (m, 1H), |
| | 7-carboxamide | | | 8.62 (s, 1H), 8.91 (s, 1H), 9.17 (br s, |
| | | | | 3H), 9.50 (br s, 1H) |
| 143 | 4-[(3S)-piperidin-3- | 342.43 | 343 | 1.81-1.57 (m, 2H), 2.10-1.91 (m, 2H), |
| | ylamino]-2-(1H-pyrazol- | | | 2.90 (m, 2H), 3.24 (m, 1H), 3.47 (m, |
| | 4-yl)thieno[3,2- | | | 1H), 4.47 (m, 1H), 7.64 (m, 1H), |
| | c]pyridine-7- | | | 7.82 (m, 1H), 8.03 (m, 3H), 8.53 (m, 1H), |
| | carboxamide | | | 8.78 (m, 2H) |
| 144 | 2-[2- | 395.49 | 396 | 1.74-1.61 (m, 2H), 2.00-1.92 (M, 2H), |
| | (aminocarbonyl)phenyl]- | | | 2.92-2.82 (m, 2H), 3.24 (m, 1H), |
| | 4-[(3S)-piperidin-3- | | | 3.48 (m, 1H), 4.51 (m, 1H), 7.60-7.42 (m, |
| | ylamino]thieno[3,2- | | | 6H), 7.75 (m, 2H), 8.01 (m, 1H), |
| | c]pyridine-7- | | | 8.53 (s, 1H), 8.67 (br s, 2H) |
| | carboxamide | | | |
| 145 | 2-(6-methoxypyridin-3- | 383.47 | 384 | 8.70 (m, 2H), 8.53 (m, 2H), 8.04 (m, 2H), |
| | yl)-4-[(3S)-piperidin-3- | | | 7.48-7.36 (m, 2H), 6.98 (d, 1H), 4.48 (m, |
| | ylamino]thieno[3,2-c]- | | | 1H), 3.90 (s, 3H), 3.31 (m, 1H), 3.27 (m, |
| | pyridine-7-carboxamide | | | 1H), 2.88 (m, 2H), 2.19-1.69 (m, 4H) |

### Example 146

### 2-(1H-indazol-1-yl)-4-[(3S)-piperidin-3-ylamino]thieno[3,2-c]pyridine-7-carboxamide

tert-butyl (3S)-3-{[7-cyano-2-(1H-indazol-1-yl)thieno[3,2-c]pyridin-4-yl]amino}piperidine-1-carboxylate. To a solution of CuI (2.7 mg, 0.014 mmol), indazole (79.2 mg, 0.670 mmol), and cesium carbonate (191 mg, 0.586 mmol) under nitrogen is added tert-butyl (3S)-3-[(7-cyano-2-iodothieno[3,2-c]pyridin-4-yl)amino]piperidine-1-carboxylate (135 mg, 0.279 mmol), trans-1,2-cyclohexanediamine (4.2 μL, 0.056 mmol) and anhydrous 1,4-dioxane (1.0 mL). The reaction mixture is stirred at 110° C. for 24 hours at which point the reaction is cooled to rt and diluted with CH₂Cl₂. The mixture is filtered and solvents are removed under reduced pressure. The black residue is purified by preparatory HPLC (5-95% MeCN, H₂O, 0.1% TFA) to afford the title compound. LCMS (ES, M+H=475).

2-(1H-indazol-1-yl)-4-[(3S)-piperidin-3-ylamino]thieno[3,2-c]pyridine-7-carboxamide. To a flask containing tert-butyl (3S)-3-{[7-cyano-2-(1H-indazol-1-yl)thieno[3,2-c]pyridin-4-yl]amino}piperidine-1-carboxylate is added 1.00 mL of 12 N HCl. The reaction mixture is stirred at rt and monitored by LCMS. Additional 12 N HCl is added every twelve hours to afford complete conversion to the desired product. Upon completion, the reaction mixture is cooled to 0° C. and treated with 6 N NaOH dropwise until a pH of 12 is obtained. The mixture is extracted with EtOAc in addition to CH₂Cl₂/MeOH (1/1), organic layers are dried over magnesium sulfate, filtered and concentrated in vacuo to yield the title compound. ¹H NMR δ 1.73-1.61 (m, 2H), 2.00-1.80 (m, 2H), 3.02-2.92 (m, 2H), 3.24 (m, 1H), 3.38 (m, 1H), 4.28 (m, 1H), 7.38 (m, 2H), 7.94 (t, 1H), 7.96 (d, 1H), 8.03 (s, 1H), 8.20 (d, 1H), 8.46 (s, 1H), 8.55 (s, 1H). LCMS (ES, M+H=393).

The following example 147 is prepared in a similar fashion.

| | | | MS | |
|---|---|---|---|---|
| | | MW | (ES, | ¹H NMR (300 MHz; d₆-DMSO; δ ppm) |
| Example | IUPAC Name | (g/mol) | M + H) | unless otherwise noted |
| 147 | 2-(1H-imidazol-1-yl)- | 342.43 | 343 | 1.82-1.70 (m, 2H), 2.03-1.94 (m, 2H), |
| | 4-[(3S)-piperidin-3- | | | 2.97-2.92 (m, 2H), 3.24 (m, 1H), 3.38 (m, |
| | ylamino]thieno[3,2- | | | 1H), 4.24 (m, 1H), 7.27 (m, 1H), 7.43 (m, |
| | c]pyridine-7- | | | 1H), 7.66 (s, 1H), 7.89 (s, 1H), 8.06 (br s, |
| | carboxamide | | | 1H), 8.16 (s, 1H), 8.58 (s, 1H) |

### Example 148

### 2-phenyl-4-[(3S)-piperidin-3-ylamino][1,3]-thiazolo[4,5-c]pyridine-7-carboxamide

2-phenyl-thiazole-5-carbaldehyde. To a solution of 2-chloromalonaldehyde (500 mg, 4.69 mmol) in 5.00 mL acetone is added thiobenzamide (643 mg, 4.69 mmol). The reaction mixture is stirred at rt until LCMS indicates the reaction is complete. The reaction mixture is concentrated under reduced pressure and the resulting solid is used directly in the next step. ¹H NMR δ 7.57 (m, 3H), 8.09 (d, 2H), 8.78 (s, 1H), 10.1 (s, 1H). LCMS (ES, M+H=190).

(2E)-3-(2-phenyl-1,3-thiazol-5-yl)acrylic acid. To 2-phenyl-thiazole-5-carbaldehyde (888 mg, 4.69 mmol) is added malonic acid (684 mg, 6.57 mmol), pyridine (0.859 mL), and piperidine (0.046 mL). The resulting mixture is heated to reflux for six hours followed by cooling to rt. The reaction mixture is poured into water (20 mL) and after stirring for ten minutes, the resultant solid is filtered, rinsed with water, and dried under reduced pressure to afford the title compound (899 mg, 83% yield). ¹H NMR δ 6.25 (d, 2H), 7.53 (m, 3H), 7.83 (d, 1H), 7.95 (m, 2H), 8.26 (s, 1H), 12.57 (br s, 1H). LCMS (ES, M+H=232).

(2E)-3-(2-phenyl-1,3-thiazol-5-yl)acryloyl azide. To a solution of (2E)-3-(2-phenyl-1,3-thiazol-5-yl)acrylic acid (899 mg, 3.89 mmol) in 15.0 mL of acetone at 0° C. is added isobutylchloroformate (0.661 mL, 5.05 mmol) drop wise. The resulting solution is stirred for one hour at 0° C. whereupon a solution of sodium azide (328 mg, 5.05 mmol) in 3.00 mL of water is added. The reaction is stirred for thirty minutes at 0° C., followed by warming to rt and stirring an additional thirty minutes. Water (50 mL) is added to the resulting solution. Filtration of the yellow solid, followed by washing with water, affords 884 mg (89% yield) of the title compound. ¹H NMR δ 6.40 (d, 1H), 7.55 (m, 3H), 7.99 (m, 2H), 8.06 (s, 1H), 8.40 (s, 1H).

2-phenyl[1,3]thiazolo[4,5-c]pyridin-4(5H)-one. To a solution of phenyl ether (3.60 mL) and tributylamine (0.900 mL) at 230° C. is added drop wise 5-[(1E)-3-oxo-3-(21⁵-triaz-1-en-2-yn-1-yl)prop-1-en-1-yl]-2-phenyl-1,3-thiazole in approximately 5.00 mL of CH₂Cl₂. The mixture is stirred at 230° C. for thirty minutes whereupon the reaction is cooled to rt, followed by the addition of 50 mL hexane to afford a yellowish solid. The resultant solid is washed with hexane and dried under reduced pressure to yield the title compound (84% yield). ¹H NMR δ 6.96 (d, 1H), 7.36 (m, 1H), 7.55 (m, 3H), 8.01 (m, 2H), 11.76 (br s, 1H).

7-bromo-2-phenyl[1,3]thiazolo[4,5-c]pyridin-4(5H)-one. To a solution of 2-phenyl[1,3]thiazolo[4,5-c]pyridin-4(5H)-one (600 mg, 2.61 mmol) in acetic acid (8.00 mL) is added bromine drop wise (0.144 mL, 2.81 mmol). The reaction mixture is heated to reflux for thirty minutes. After thirty minutes, the solution is cooled to rt, and 40 mL of water is added. The remaining solid is filtered, rinsed with water, and dried under reduced pressure to afford the title compound (728 mg, 90% yield). ¹H NMR δ 7.56 (m, 3H), 7.72 (s, 1H), 8.06 (m, 2H). LCMS (ES, M+H=309).

4-chloro-2-phenyl[1,3]thiazolo[4,5-c]pyridine-7-carbonitrile. To 7-bromo-2-phenyl[1,3]thiazolo[4,5-c]pyridin-4(5H)-one (728 mg, 2.35 mmol) in approximately 10.0 mL of N,N-dimethylformamide (DMF) is added copper(I) cyanide (464 mg, 5.18 mmol). The reaction is stirred at reflux for ten hours followed by cooling to rt. A solution of iron(III) chloride (4.57 g, 28.2 mmol) dissolved in 1.30 mL of concentrated HCl and 7.30 mL of water is then added. The mixture is stirred for fifteen minutes at 70° C., followed by cooling to rt. Water (40.0 mL) is added and the solid is filtered and dried under reduced pressure. The resulting solid is treated with 7.00 mL of phosphorus oxychloride and set to reflux for four hours whereupon the reaction is cooled to rt. The solvents are removed in vacuo. The residue is dissolved in CH₂Cl₂, washed with saturated NaHCO₃, and the organic layers dried with MgSO₄, filtered, and concentrated under reduced pressure. The resulting solid is purified by silica gel chromatography (100% CH₂Cl₂) to afford the title compound (189 mg, 30% yield). ¹H NMR δ 7.83-7.74 (m, 3H), 8.22 (d, 2H), 8.95 (s, 1H). LCMS (ES, M−H=272).

tert-butyl (3S)-3-[(7-cyano-2-phenyl[1,3]thiazolo[4,5-c]pyridin-4-yl)amino]piperidine-1-carboxylate. To a solution of 4-chloro-2-phenyl[1,3]thiazolo[4,5-c]pyridine-7-carbonitrile (189 mg, 0.690 mmol) in NMP (3.0 mL) is added potassium carbonate (229 mg, 1.66 mmol) and tert-butyl (3S)-3-aminopiperidine-1-carboxylate (691 mg, 3.45 mmol). The reaction mixture is heated to 100° C. until LCMS indicated the completion of the reaction. The reaction mixture is then filtered, affording a viscous oil which is purified by silica gel column chromatography (100% hexane to 100% EtOAc) and concentrated to dryness to yield 255 mg of the title compound (85% yield). ¹H NMR δ 1.35 (s, 9H), 1.96-1.69 (m, 4H), 2.90 (m, 2H), 4.07-3.66 (m, 2H), 4.16 (m, 1H), 7.61 (m, 3H), 7.96 (br s, 1H), 8.15 (m, 2H), 8.49 (s, 1H). LCMS (ES, M+H=436).

2-phenyl-4-[(3S)-piperidin-3-ylamino][1,3]thiazolo[4,5-c]pyridine-7-carboxamide. To tert-butyl (3S)-3-[(7-cyano-2-phenyl[1,3]thiazolo[4,5-c]pyridin-4-yl)amino]piperidine-1-carboxylate (255 mg) is added 3.00 mL of 12N HCl. The cloudy solution is stirred at rt and monitored for completion by LCMS. The reaction mixture is cooled to 0° C. and treated with 6N NaOH drop wise until a pH of 12 is obtained. The mixture is extracted with EtOAc in addition to CH₂Cl₂/MeOH (1/1), organic layers are dried over magnesium sulfate, filtered and concentrated in vacuo to yield product which is purified by preparatory HPLC (5-95% MeCN, H₂O, 0.1% TFA) to afford 100 mg of the title compound (48% yield). ¹H NMR δ 1.99-1.65 (m, 4H), 2.91-2.83 (m, 2H), 3.24 (m, 1H), 3.38 (m, 1H), 4.56 (m, 1H), 5.2-6.2 (br s, 2H), 7.47 (m, 3H), 7.65 (d, 1H), 8.12 (m, 2H), 8.67-8.61 (overlapping m and s, 2H). LCMS (ES, M+H=354).

The following examples 149-155 are prepared in an analogous fashion to example 148 using the appropriate starting materials.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 149 | 4-[(2-aminoethyl)amino]-2- | 343.41 | 344 | (400.132 MHz) 2.93 (m, 2H), |
| | (4- | | | 3.66 (m, 2H), 3.86 (s, 3H), 7.12 (d, 2H), |
| | methoxyphenyl)[1,3]thiazolo[4, | | | 7.44 (br s, 1H), 7.67 (br t, 1H), |
| | 5-c]pyridine-7- | | | 8.06 (br s, 3H), 8.09 (d, 3H), |
| | carboxamide | | | 8.61 (s, 1H) |
| 150 | 4-{[2- | 371.46 | 372 | (400.132 MHz) 2.23 (s, 6H), |
| | (dimethylamino)ethyl]amino}- | | | 2.55 (t, 2H), 3.67 (dt, 2H), 3.86 (s, 3H), |
| | 2-(4- | | | 7.12 (d, 2H), 7.37 (t, 1H), 7.39 (br |
| | methoxyphenyl)[1,3]thiazolo[4, | | | s, 1H), 8.00 (br s, 1H), 8.07 (d, |
| | 5-c]pyridine-7- | | | 2H), 8.62 (s, 1H) |
| | carboxamide | | | |
| 151 | 2-(4-methoxyphenyl)-4- | 383.47 | 384 | (400.132 MHz) 1.56 (m, 2H), |
| | (piperidin-4- | | | 1.90 (m, 2H), 2.59 (m, 2H), 3.01 (m, |
| | ylamino)[1,3]thiazolo[4,5- | | | 2H), 3.86 (s, 3H), 4.22 (m, 1H), |
| | c]pyridine-7-carboxamide | | | 7.11 (d, 2H), 7.20 (d, 1H), 7.38 (br |
| | | | | s, 1H), 7.97 (br s, 1H), 8.11 (d, |
| | | | | 2H), 8.59 (s, 1H) |
| 152 | 2-(4-methoxyphenyl)-4- | 383.47 | 384 | (400.132 MHz) 1.50 (m, 1H), |
| | [(3S)-piperidin-3- | | | 1.69 (m, 2H), 1.70 (m, 2H), 1.91 (m, |
| | ylamino][1,3]thiazolo[4,5- | | | 1H), 2.57 (m, 1H), 2.64 (m, 1H), |
| | c]pyridine-7-carboxamide | | | 2.82 (m, 1H), 3.09 (m, 1H), |
| | | | | 3.86 (s, 3H), 4.26 (m, 1H), 7.12 (d, 2H), |
| | | | | 7.17 (d, 1H), 7.38 (br s, 1H), |
| | | | | 8.00 (br s, 1H), 8.09 (d, 2H), 8.60 (s, |
| | | | | 1H) |
| 153 | 4-[(3-aminopropyl)amino]- | 357.44 | 358 | (400.132 MHz) 1.73 (m, 2H), |
| | 2-(4- | | | 1.76 (br s, 2H), 2.66 (t, 2H), 3.64 (dt, |
| | methoxyphenyl)[1,3]thiazolo[4, | | | 2H), 3.86 (s, 3H), 7.12 (d, 2H), |
| | 5-c]pyridine-7- | | | 7.36 (br s, 1H), 7.74 (t, 1H), |
| | carboxamide | | | 7.97 (br s, 1H), 8.08 (d, 2H), 8.60 (s, |
| | | | | 1H) |
| 154 | 2-(4-methoxyphenyl)-4- | 369.45 | 370 | (400.132 MHz) 2.11 (m, 1H), |
| | (pyrrolidin-3- | | | 2.55 (m, 1H), 2.81 (m, 2H), 3.00 (m, |
| | ylamino)[1,3]thiazolo[4,5- | | | 1H), 3.07 (dd, 1H), 3.86 (s, 3H), |
| | c]pyridine-7-carboxamide | | | 4.68 (m, 1H), 7.11 (d, 2H), 7.35 (d, |
| | | | | 1H), 7.38 (br s, 1H), 8.00 (br s, |
| | | | | 1H), 8.10 (d, 2H), 8.62 (s, 1H) |
| 155 | 4-(azetidin-3-ylamino)-2- | 355.42 | 356 | (400.132 MHz) 3.71 (d, 4H), |
| | (4- | | | 3.87 (s, 3H), 5.01 (m, 1H), 7.13 (d, 2H), |
| | methoxyphenyl)[1,3]thiazolo[4, | | | 7.41 (br s, 1H), 7.90 (d, 1H), |
| | 5-c]pyridine-7- | | | 8.00 (br s, 1H), 8.12 (d, 2H), 8.60 (s, |
| | carboxamide | | | 1H) |

### Example 156

### 2-phenyl-4-[(3S)-piperidin-3-ylamino]furo[3,2-c]pyridine-7-carboxamide

(2E)-3-(5-phenyl-2-furyl)acrylic acid. 5-phenyl-2-furylaldehyde (2.82 g, 16.4 mmol) is treated with malonic acid (2.4 g, 23.0 mmol), pyridine (3 ml) and piperidine (0.16 ml). The mixture is heated at reflux for 6 hours before being cooled to rt. The mixture is then poured into water (50 ml) with stirring. The resultant yellow solid is filtered, washed with water and air dried to give the title compound (3.5 g, 99%). ¹H NMR δ 12.39 (br s, 1H), 7.83 (d, 2H), 7.47 (t, 2H), 7.38 (t, 2H), 7.13 (d, 1H), 7.05 (d, 1H), 6.33 (d, 1H). LCMS (ES, M+H=215).

(2E)-3-(5-phenyl-2-furyl)acryloyl azide. To a solution of (2E)-3-(5-phenyl-2-furyl)acrylic acid (1.63 g, 7.6 mmol) and Et₃N (1.40 ml, 9.9 mmol) in acetone (20 ml) at 0° C. is added drop wise ClCO₂iBu (1.3 ml, 9.9 mmol). After stirring for 1 h at 0° C., sodium azide (643 mg, 9.9 mmol) in water (5 ml) is added and the resultant mixture is stirred at 0° C. for a further 30 min and then at rt for 30 min before the addition of water (100 ml). Filtration gives the title compound as a yellow solid, which is washed with water and air dried (1.21 g, 67%). ¹H NMR δ 7.88 (d, 2H), 7.60 (d, 1H), 7.48 (t, 2H), 7.39 (t, 1H), 7.22 (d, 2H), 6.44 (d, 1H). LCMS (ES, M+H=240).

2-phenylfuro[3,2-c]pyridin-4(5H)-one. To a stirred mixture of phenyl ether (36.4 ml) and Bu₃N (9.1 ml) at 230° C. is added drop wise a solution of (2E)-3-(5-phenyl-2-furyl)acryloyl azide (2.29 g, 9.56 mmol) in CH₂Cl₂ (18 ml). The addition rate is controlled such that the internal temperature remained above 190° C. After addition, the resulting brown solution is stirred for 30 min before cooling to rt. Hexanes (90 ml) is added and the yellow solid is filtered, washed with hexane and dried in the air to afford the title compound (1.3 g, 64.5%). ¹H NMR δ 11.51 (s, 1H), 7.84 (d, 2H), 7.48 (t, 3H), 7.37 (m, 2H), 6.70 (d, 1H). LCMS (ES, M+H=212).

7-bromo-2-phenylfuro[3,2-c]pyridin-4(5H)-one. A solution of 2-phenylfuro[3,2-c]pyridin-4(5H)-one (369 mg, 1.75 mmol) in acetic acid (5 ml) at rt is treated with bromine (320 mg, 1.93 mmol) and the resulting mixture is heated at reflux for 30 min. After cooling to rt, water (20 ml) is added to the mixture. The yellow solid which formed is filtered, washed with water and dried in the air to afford a mixture of compound the title compound and 6,7-dibromo-2-phenylfuro[3,2-c]pyridin-4(5H)-one (2:1, 400 mg, 52%) which is used directly in the next step.

4-oxo-2-phenyl-4,5-dihydrofuro[3,2-c]pyridine-7-carbonitrile. A mixture of 7-bromo-2-phenylfuro[3,2-c]pyridin-4(5H)-one (472 mg, 1.63 mmol) and CuCN (320 mg, 3.58 mmol) in DMF is heated at reflux for 16 hours before cooling to rt. A solution of FeCl₃ (3.32 g, 20 mmol) in concentrated HCl (0.9 ml) and water (5 ml) is then added to decompose the copper complex. The mixture is stirred at 70° C. for 15 min and then allowed to cool to rt. Water (35 ml) is added and a yellow solid is formed which is filtered, washed with water and dried in the air. The mixture of the title compound and 4-oxo-2-phenyl-4,5-dihydrofuro[3,2-c]pyridine-6,7-dicarbonitrile is carried on to the next step without purification.

4-chloro-2-phenylfuro[3,2-c]pyridine-7-carbonitrile. Crude 4-oxo-2-phenyl-4,5-dihydrofuro[3,2-c]pyridine-7-carbonitrile is treated with POCl₃ (5 ml) and the mixture heated at reflux for 4 hours. The solvent is removed under reduced pressure and the residue is partitioned between CH₂Cl₂ and aqueous sodium bicarbonate solution. The organic phase is separated and dried over magnesium sulfate. Removal of solvent followed by silica gel column chromatography (eluent: CH₂Cl₂ and MeOH) gave the title compound as a white solid (287 mg, 69.7% for two steps). ¹H NMR δ 8.35 (s, 1H), 8.06 (d, 2H), 7.86 (s, 1H), 7.57 (m, 3H). LCMS (ES, M+H=254).

tert-butyl (3S)-3-[(7-cyano-2-phenylfuro[3,2-c]pyridin-4-yl)amino]piperidine-1-carboxylate. To a mixture of 4-chloro-2-phenylfuro[3,2-c]pyridine-7-carbonitrile (287 mg, 1.13 mmol) and potassium carbonate (376 mg, 2.72 mmol) in NMP (5 ml), is added tert-butyl(3S)-3-aminopiperidine-1-carboxylate (1.14 g, 5.67 mmol), and the resulting mixture is stirred at 110° C. for 16 hours. The mixture is then cooled and water (50 ml) is added and the precipitate that is formed is filtered to give the title compound (178 mg).

2-phenyl-4-[(3S)-piperidin-3-ylamino]furo[3,2-c]pyridine-7-carboxamide. tert-butyl

(3S)-3-[(7-cyano-2-phenylfuro[3,2-c]pyridin-4-yl)amino]piperidine-1-carboxylate (178 mg). is treated with conc. HCl (5 ml) and the mixture is stirred at rt overnight. After removal of the solvent, the residue is purified by preparatory HPLC to give the title compound as a trifluoroacetate salt. The salt is dissolved in MeOH (1 ml), and then charged with 4N HCl/Dioxane (2 ml). After stirring overnight, the white solid is filtered and dried in the air to afford the title compound (53 mg). ¹H NMR δ 9.14 (br s, 1H), 8.89 (brs, 1H), 8.34 (s, 1H), 7.91 (d, 2H), 7.85 (s, 1H), 7.67 (s, 2H), 7.55 (t, 2H), 7.45 (t, 1H), 4.47 (m, 1H), 3.19 (m, 2H), 2.96 (m, 2H), 2.03 (m, 2H), 1.72 (m, 2H). LCMS (ES, M+H=337).

### Example 157

### 2-methyl-4-[(3S)-piperidin-3-ylamino]thieno[3,2-c]pyridine-7-carboxamide

(2E)-3-(5-methyl-2-thienyl)acrylic acid. To 5-methylthiophene-2-carbaldehyde (13.1 mL, 120 mmol) is added malonic acid (17.5 g, 168 mmol), pyridine (22.0 mL), and piperidine (1.18 mL). The resulting mixture is heated to reflux overnight followed by cooling to rt. The reaction mixture is then poured into water (200 mL) and after stirring for ten minutes, the resultant solid is filtered, rinsed with water, and dried under reduced pressure to afford the title compound (13.2 g, 66% yield). ¹H NMR δ 12.3 (s, 1H), 7.68 (d, 1H), 7.32 (s, 1H), 6.86 (s, 1H), 6.03 (d, 1H), 3.36 (s, 3H). LCMS (ES, M+H=169).

(2E)-3-(5-methyl-2-thienyl)acryloyl azide. To a solution of (2E)-3-(5-methyl-2-thienyl)acrylic acid (13.2 g, 78.3 mmol) in 300 mL of acetone at 0° C. is added isobutylchloroformate (13.3 mL, 102 mmol) drop wise. The resulting solution is stirred for one hour at 0° C. whereupon a solution of sodium azide (6.63 g, 102 mmol) in 64.0 mL of water is added. The reaction is then stirred for thirty minutes at 0° C., followed by warming to rt and stirring an additional thirty minutes. Water (500 mL) is added to the resulting solution. Filtration of the yellow solid, which is washed with water, afforded the title compound (83% yield). ¹H NMR δ 7.85 (d, 1H), 7.47 (s, 1H), 6.91 (s, 1H), 6.14 (d, 1H), 3.32 (s, 3H).

2-methylthieno[3,2-c]pyridin-4(5H)-one. To a solution of phenyl ether (149 mL) and tributylamine (37.0 mL) at 230° C. is added drop wise (2E)-3-(5-methyl-2-thienyl)acryloyl azide (7.60 g, 39.3 mmol) in approximately 5.00 mL of CH₂Cl₂. The mixture is stirred at 230° C. for thirty minutes whereupon the reaction is cooled to rt, followed by the addition of 200 mL hexane to afford a yellowish solid. The resultant solid is washed with hexane and dried under reduced pressure to yield the title compound (4.84 g, 74% yield). ¹H NMR δ 11.3 (s, 1H), 7.14 (s, 2H), 6.73 (d, 1H), 3.32 (s, 3H).

7-bromo-2-methylthieno[3,2-c]pyridin-4(5H)-one. To a solution of 2-methylthieno[3,2-c]pyridin-4(5 μl)-one (4.84 g, 28.9 mmol) in acetic acid (84.0 mL) is added bromine drop wise (1.64 mL, 31.8 mmol). The reaction mixture is heated to reflux for one hour. After one hour, the solution is cooled to rt, and water is added until a solid is formed. The remaining solid is filtered, rinsed with water, and dried under vacuum to afford the title compound (6.01 g, 85% yield). ¹H NMR δ 11.7 (br s, 1H), 7.47 (s, 1H), 7.30 (s, 1H), 3.38 (s, 3H). LCMS (ES, M+H=245).

4-chloro-2-methylthieno[3,2-c]pyridine-7-carbonitrile. To a solution of 7-bromo-2-methylthieno[3,2-c]pyridin-4(5H)-one (2.76 g, 11.3 mmol) in approximately 24.0 mL of N,N-dimethylformamide (DMF) is added copper(I) cyanide (2.22 g, 24.9 mmol). The reaction is stirred at reflux for ten hours followed by cooling to rt. A solution of iron(III) chloride (11.0 g, 67.8 mmol) dissolved in 6.30 mL of concentrated HCl and 35.0 mL of water is then added. The mixture is stirred for fifteen minutes at 70° C., followed by cooling to rt. Water (192 mL) is added and the solid is filtered and dried under reduced pressure. The resulting solid is then treated with 34.0 mL of phosphorus oxychloride and set to reflux for four hours whereupon the reaction is cooled to rt. The solvents are removed in vacuo. The residue is dissolved in CH₂Cl₂, washed with saturated NaHCO₃, and the organic layers dried with MgSO₄, filtered, and concentrated under reduced pressure to afford the title compound (943 mg, 40% yield). ¹H NMR δ 8.78 (s, 1H), 7.49 (s, 1H), 3.33 (s, 3H). LCMS (ES, M+H=209).

tert-butyl (3S)-3-[(7-cyano-2-methylthieno[3,2-c]pyridin-4-yl)amino]piperidine-1-carboxylate. To a solution of 4-chloro-2-methylthieno[3,2-c]pyridine-7-carbonitrile (943 mg, 4.52 mmol) in NMP (5.0 mL) is added potassium carbonate (1.49 g, 10.8 mmol) and tert-butyl (3S)-3-aminopiperidine-1-carboxylate (2.72 g, 13.6 mmol). The reaction mixture is heated to 130° C. until LCMS indicated the completion of the reaction. The reaction mixture is cooled to rt and approximately 100 mL of water is added. The resulting solid is filtered and vacuum dried to afford the title compound. LCMS (ES, M+Na=395).

2-methyl-4-[(3S)-piperidin-3-ylamino]thieno[3,2-c]pyridine-7-carboxamide. To a flask containing tert-butyl (3S)-3-[(7-cyano-2-methylthieno[3,2-c]pyridin-4-yl)amino]piperidine-1-carboxylate is added 5.00 mL of 12 N HCl. The reaction mixture is stirred at rt and monitored by LCMS. Additional 12 N HCl is added every twelve hours to afford complete conversion to the desired product. Upon completion, the reaction mixture is diluted with water and concentrated under reduced pressure to yield product, which is purified by silica gel chromatography (100% CH₂Cl₂ to 20% MeOH/CH₂Cl₂/3% NH₄OH) to afford the title compound. ¹H NMR δ 8.42 (s, 1H), 7.81 (br s, 1H), 7.41 (s, 1H), 7.14 (s, 1H), 7.14 (br s, 1H), 6.96 (d, 1H), 4.11 (m, 1H), 3.31 (s, 3H), 3.14 (m, 2H), 2.82 (m, 2H), 1.94 (m, 1H), 1.52 (m, 1H), 1.36 (m, 2H). LCMS (ES, M+H=291).

### Example 158

### 2-(3-fluorophenyl)-7-[(3S)-piperidin-3-ylamino]thieno[2,3-c]pyridine-4-carboxamide

(2Z)-3-cyano-3-(3-thienyl)acrylic acid. To 3-thiopheneacetonitrile (166 mmol) is added glyoxylic acid (174 mmol), MeOH (332 mL) and potassium carbonate (174 mmol). The resulting mixture is heated to reflux for three hours followed by cooling to rt. The resultant solid is filtered, rinsed with MeOH, and dried in a vacuum oven to afford the title compound (26.6 g, 90% yield). LCMS (ES, M−H=178).

(2Z)-3-cyano-3-(3-thienyl)acryloyl chloride. To a solution of oxalyl chloride (27.3 mL, 313 mmol) in CH₂Cl₂ (57 mL) is added (2Z)-3-cyano-3-(3-thienyl)acrylic acid (26.6 g, 149 mmol) in portions. The resulting solution is stirred at rt until LCMS indicated completion of the reaction. The reaction mixture is then filtered and rinsed with CH₂Cl₂. The filtrate is collected, concentrated under reduced pressure and dried under vacuum to afford the title compound as a yellow solid which is used directly in the next reaction (18.5 g, 63% yield).

(2Z)-3-cyano-3-(3-thienyl)acryloyl azide. To a solution of sodium azide (12.2 g, 187 mmol) in a 1:1 mixture of dioxane/water (23 mL) is added at 0° C. (2Z)-3-cyano-3-(3-thienyl)acryloyl chloride (18.5 g, 93.5 mmol) in 33 mL dioxane. The reaction is stirred for 15 minutes at 0° C., followed by warming the reaction to rt. After approximately 1.5 hours, water (100 mL) is added to the reaction and the resulting solid is filtered and dried in a vacuum oven to yield the title compound (15.1 g, 82% yield). ¹H NMR δ 8.24 (s, 1H), 7.76-7.71 (m, 2H), 7.25 (s, 1H). LCMS (ES, M−H=204).

7-oxo-6,7-dihydrothieno[2,3-c]pyridine-4-carbonitrile. To a solution of phenyl ether (224 mL) and tributylamine (53.0 mL) at 230° C. is added drop wise (2Z)-3-cyano-3-(3-thienyl)acryloyl azide in approximately 10 mL of CH₂Cl₂. The mixture is stirred at 230° C. for thirty minutes, cooled to rt, followed by the addition of 500 mL hexane, which affords a yellowish solid. The resultant solid is washed with hexane and dried under vacuum to yield the title compound (4.61 g, 44% yield). ¹H NMR δ 12.4 (br s, 1H), 8.26 (m, 2H), 7.42 (d, 1H).

2-bromo-7-oxo-6,7-dihydrothieno[2,3-c]pyridine-4-carbonitrile. To a solution of 7-oxo-6,7-dihydrothieno[2,3-c]pyridine-4-carbonitrile (2.30 g, 13.1 mmol) in 1/1 acetic acid/DMF (10 mL) is added N-bromosuccinimide (11.6 g, 65.3 mmol). The reaction mixture is heated to 80° C. for one. The solution is cooled to rt and diluted with 100 mL of water. The reaction is then neutralized with saturated sodium bicarbonate followed by filtration of the resulting solid, which is dried in a vacuum oven to afford the title compound (3.20 g, 96% yield). ¹H NMR δ 12.7 (br s, 1H), 8.41 (s, 1H), 8.32 (d, 1H). LCMS (ES, M+H=256).

2-bromo-7-chlorothieno[2,3-c]pyridine-4-carbonitrile. To 2-bromo-7-oxo-6,7-dihydrothieno[2,3-c]pyridine-4-carbonitrile (3.20 g, 12.5 mmol) is added 45.0 mL of phosphorous oxychloride. The reaction is heated to reflux overnight after which LCMS indicated reaction is complete. The reaction is then cooled to rt and the volatiles are removed under reduced pressure. To the resulting residue is added approximately 200 mL of water. The black solid is filtered and rinsed with copious amounts of water and dried under vacuum to yield the title compound (2.80 g, 82% yield). ¹H NMR δ 8.97 (s, 1H), 8.71 (s, 1H).

tert-butyl (3S)-3-[(2-bromo-4-cyanothieno[2,3-c]pyridin-7-yl)amino]piperidine-1-carboxylate. To a solution of 2-bromo-7-chlorothieno[2,3-c]pyridine-4-carbonitrile (2.80 g, 10.2 mmol) in NMP (10.0 mL) is added potassium carbonate (4.23 g, 30.6 mmol) and tert-butyl (3S)-3-aminopiperidine-1-carboxylate (4.92 g, 24.6 mmol). The reaction mixture is heated to 130° C. until LCMS indicates the reaction is complete. The reaction mixture is then cooled to rt and approximately 100 mL of water is added. The resulting solid is filtered and vacuum dried to afford the title compound. ¹H NMR δ 8.47 (s, 1H), 8.35 (s, 1H), 7.90 (br s, 1H), 4.14 (m, 1H), 3.38 (m, 1H), 3.24 (m, 1H), 2.93 (m, 2H), 1.94-1.73 (m, 4H), 1.37 (s, 9H). LCMS (ES, M+H=338).

tert-butyl (3S)-3-{[4-cyano-2-(3-fluorophenyl)thieno[2,3-c]pyridin-7-yl]amino}piperidine-1-carboxylate. To tert-butyl (3S)-3-[(2-bromo-4-cyanothieno[2,3-c]pyridin-7-yl)amino]piperidine-1-carboxylate (428 mg, 0.979 mmol) is added cesium carbonate (957 mg, 2.94 mmol), 3-fluorophenyl boronic acid (206 mg, 1.47 mmol), Pd(PPh₃)₄ (113 mg, 0.0979 mmol), and dioxane/water (4 mL/2 mL). The reaction is heated to 80° C. for one hour whereupon the reaction is cooled to rt, filtered, and purified using silica gel chromatography (100% hexanes to 100% EtOAc) to afford the title compound (241 mg, 54% yield). LCMS (ES, M+H=453).

2-(3-fluorophenyl)-7-[(3S)-piperidin-3-ylamino]thieno[2,3-c]pyridine-4-carboxamide. To a flask containing tert-butyl (3S)-3-{[4-cyano-2-(3-fluorophenyl)thieno[2,3-c]pyridin-7-yl]amino}piperidine-1-carboxylate is added approximately 2.00 mL of PPA. The reaction mixture is stirred at 110° C. for 12 hours. The reaction mixture is diluted with 10.0 mL of water and brought to a basic pH with 6N NaOH. The mixture is then extracted with EtOAc (4×100 mL) followed by CH₂Cl₂/MeOH (1/1, 4×100 mL), dried over MgSO₄, and concentrated under reduced pressure to yield the product that is purified by silica gel chromatography (100% CH₂Cl₂ to 20% MeOH/CH₂Cl₂/3% NH₄OH) to afford the title compound. ¹H NMR δ 8.01 (s, 1H), 7.91 (s, 1H), 7.51 (s, 1H), 7.38 (m, 1H), 7.36 (m, 4H), 7.34 (br s, 1H), 6.80 (m, 1H), 4.21 (m, 1H), 3.15 (m, 2H), 2.87 (m, 2H), 1.92-1.46 (m, 4H). LCMS (ES, M+H=371).

The following example 159 is prepared in an analogous fashion using the appropriate starting materials.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 159 | 2-phenyl-7-[(3S)- | 352.46 | 353 | 8.01 (s, 1H), 7.86 (s, 1H), 7.68 (m, 1H), |
| | piperidin-3- | | | 7.49-7.24 (m, 5H), 7.12 (br s, 1H), |
| | ylamino]thieno[2,3- | | | 4.50 (m, 1H), 3.3-3.8 (br s, 2H), 3.15 (m, |
| | c]pyridine-4- | | | 2H), 2.87 (m, 2H), 2.01-1.20 (m, 4H) |
| | carboxamide | | | |

### Example 160

### 2-phenyl-4-(piperidin-3-ylamino)-1H-indole-7-carboxamide

methyl 2-amino-4-nitrobenzoate. To a solution of 2-amino-4-nitrobenzoic acid (24 g, 0.132 mol) in MeOH (500 mL) is slowly added thionyl chloride (96 mL). The resulting solution is refluxed overnight. Upon cooling, the crystalline product is isolated by filtration and drying under high vacuum (22.9 g, 88%). ¹H NMR δ 7.90 (d, 1H) 7.67 (d, 1H) 7.25 (dd, 1H) 7.13 (s, 2H) 3.84 (s, 3H).

4-nitro-2-phenyl-1H-indole-7-carboxylic acid. To a solution of methyl 2-amino-4-nitrobenzoate (2.2 g, 11.2 mmol) and acetophenone (2.8 g, 23.3 mmol) in DMSO (30 mL) cooled to −15° C. is added solid KOtBu (2.7 g, 24 mmol). After stirring for 20 min., and then another 2 h at rt, the reaction is quenched with sat. NH₄Cl (200 mL) and then stirred for an additional 1 h at rt. The red precipitate is filtered, washed with water, and dried under high vacuum to give the title compound (2.85 g, 90%). ¹H NMR δ 12.05 (s, 1H) 7.99 (d, 1H) 7.89 (d, 2H) 7.65 (d, 1H) 7.50 (t, 2H) 7.44 (s, 1H) 7.41 (d, 1H) 7.30 (br s, 1H). LCMS (ES, M−H=281).

4-nitro-2-phenyl-1H-indole-7-carboxamide. To a solution of 4-nitro-2-phenyl-1H-indole-7-carboxylic acid (0.60 g, 2.1 mmol) and N-methylmorpholine (2.3 mmol) in CH₂Cl₂ (20 mL) at −15° C. is added isobutyl chloroformate (0.5 mL, 3.8 mmol). After stirring for 1 h, NH₃ (g) is bubbled through the reaction mixture for 10-15 min. and then stirred for an additional 1 h at rt. After removing the solvent, the residue is purified by MPLC (SiO₂; 50-100% EtOAc/Hexanes) to give the product as a dark yellow solid (0.50 g, 85%). ¹H NMR δ 11.72 (s, 1H) 8.46 (s, 1H) 8.11 (d, 1H) 8.02 (d, 2H) 7.92 (s, 1H) 7.76 (d, 1H) 7.50-7.57 (m, 3H) 7.47 (d, 1H). LCMS (ES, M+H=282; M−H=280).

4-amino-2-phenyl-1H-indole-7-carboxamide. To a nitrogen-purged stirred solution of 4-nitro-2-phenyl-1H-indole-7-carboxamide (0.50 g, 17.8 mmol) dissolved in MeOH (30 mL) is added 10% Pd/C (30 mg). The resultant heterogeneous mixture is affixed with a H₂ (g) balloon. After stirring overnight at rt, the reaction is filtered (0.45 u, Teflon). The filtrate is concentrated in vacuo to give the title compound as a light yellow solid (0.35 g, 80%). ¹H NMR δ 10.90 (s, 1H) 7.74 (d, 3H) 7.53 (q, 4H) 7.35 (t, 1H) 7.12 (d, 1H) 6.22 (d, 1H) 6.10 (s, 2H). LCMS (ES, M+H=252; M−H=250).

tert-butyl 3-{[7-(aminocarbonyl)-2-phenyl-1H-indol-4-yl]amino}piperidine-1-carboxylate

To a solution of 4-amino-2-phenyl-1H-indole-7-carboxamide (0.60 g, 2.4 mmol) and tert-butyl 3-oxopiperidine-1-carboxylate (0.6 g, 2.8 mmol) dissolved in AcOH (15 mL) is added Na₂SO₄. The mixture is stirred at rt for 1 h and then slowly charged with sodium triacetoxyborohydride (1.5 g, 7.2 mmol). The reaction is stirred at rt for 1 h. The mixture is diluted with EtOAc and water, washed with sat. NaHCO₃, 1N HCl, and sat. NaCl. The organic layer is dried over Na₂SO₄, filtered, and CIV. The residue is purified by MPLC (SiO₂; 50-80% EtOAc/Hexanes title product as a tan solid (0.3 g, 30%). LCMS (ES, M+H=435; M−H=433).

2-phenyl-4-(piperidin-3-ylamino)-1H-indole-7-carboxamide. A stirred solution of tert-butyl 3-{[7-(aminocarbonyl)-2-phenyl-1H-indol-4-yl]amino}piperidine-1-carboxylate (0.15 g, 0.35 mmol) in MeOH (10 mL) is charged with 4.0 N HCl in dioxane (10 mL). The reaction is stirred for 2 h at rt and then concentrated in vacuo to give the hydrochloride salt. The residue is diluted with 2.0 N NH₃ in MeOH (10 mL) and CIV. The residue is purified by MPLC (SiO₂; 10% MeOH/CH₂Cl₂/1.5% NH₄OH-20% MeOH/CH₂Cl₂/3% NH₄OH) to give the title compound as an off-white solid (90 mg, 78%). ¹H NMR δ 10.87 (s, 1H) 7.69 (d, 2H) 7.57 (d, 1H) 7.45 (t, 2H) 7.28 (t, 1H) 7.20 (s, 1H) 6.96 (br s, 1H) 6.15 (d, 1H) 6.02 (d, 1H) 3.50 (d, 1H) 3.30 (s, 2H) 3.05-3.20 (m, 2H) 2.84 (d, 1H) 2.34-2.46 (m, 1H) 1.98 (s, 1H) 1.60-1.72 (m, 1H) 1.43-1.58 (m, 2H). LCMS (ES, M+H=335; M−H=333).

The following examples 161-169 are prepared in an analogous fashion to example 158 using the appropriate starting materials.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 161 | 2-(4-chlorophenyl)-4- | 368.87 | 369 | 10.75 (s, 1H), 9.10 (m, 2H), 7.55 (d, |
| | (piperidin-3-ylamino)- | | | 2H), 7.45 (d, 1H), 7.33 (d, 2H), 7.15 (d, |
| | 1H-indole-7-carboxamide | | | 1H), 6.08 (d, 1H), 5.45 (br s, 3H), |
| | | | | 3.83 (m, 1H), 3.21 (m, 1H), 3.03 (m, 1H), |
| | | | | 2.73 (m, 1H), 2.65 (m, 1H), 1.80 (m, |
| | | | | 2H), 1.62 (m, 1H), 1.51 (m, 1H). |
| 162 | 2-(4-fluorophenyl)-4- | 352.41 | 353 | 10.89 (s, 1H), 8.92 (m, 1H), 8.73 (m, |
| | (piperidin-3-ylamino)- | | | 1H), 7.75 (d, 1H), 7.74 (d, 1H), 7.60 (d, |
| | 1H-indole-7-carboxamide | | | 1H), 7.30 (t, 2H), 7.15 (d, 1H), 7.04 (br |
| | | | | s, 1H), 6.33 (br s, 1H), 6.23 (d, 1H), |
| | | | | 3.91 (m, 1H), 3.41 (m, 1H), 3.25 (m, 1H), |
| | | | | 2.89 (m, 1H), 2.75 (m, 1H), 2.03 (m, |
| | | | | 1H), 1.94 (m, 1H), 1.74 (m, 1H), |
| | | | | 1.66 (m, 1H). |
| 163 | 2-(4-methoxyphenyl)-4- | 364.45 | 365 | 10.8 (s, 1H), 9.06 (m, 1H), 8.95 (m, 1H), |
| | (piperidin-3-ylamino)- | | | 7.63 (m, 3H), 7.57 (d, 1H), 7.05 (m, 3H), |
| | 1H-indole-7-carboxamide | | | 6.22 (d, 1H), 3.79 (s, 3H), 3.68 (m, 2H), |
| | | | | 3.43 (m, 2H), 3.23 (m, 1H), 2.89 (m, |
| | | | | 1H), 2.75 (m, 1H), 2.03 (m, 1H), |
| | | | | 1.94 (m, 1H), 1.74 (m, 1H), 1.66 (m, 1H). |
| 164 | 2-[4- | 377.49 | 378 | 10.84 (s, 1H), 9.24 (m, 2H), 7.71 (m, |
| | (dimethylamino)phenyl]- | | | 2H), 7.58 (d, 1H), 7.33 (m, 2H), 7.19 (m, |
| | 4-(piperidin-3-ylamino)- | | | 1H), 6.24 (d, 1H), 3.99 (m, 2H), 3.68 (m, |
| | 1H-indole-7-carboxamide | | | 1H), 3.47 (m, 1H), 3.38 (m, 1H), |
| | | | | 3.20 (m, 1H), 3.04 (s, 6H), 2.89 (m, 1H), |
| | | | | 2.78 (m, 1H), 1.94 (m, 2H), 1.74 (m, 1H), |
| | | | | 1.66 (m, 1H). |
| 165 | 2-(3-chlorophenyl)-4- | 368.87 | 369 | 10.98 (s, 1H), 9.19 (m, 1H), 9.08 (m, |
| | (piperidin-3-ylamino)- | | | 1H), 7.79 (s, 1H), 7.66 (dd, 2H), 7.49 (t, |
| | 1H-indole-7-carboxamide | | | 1H), 7.37 (s, 2H), 7.34 (s, 1H), 6.31 (br |
| | | | | s, 2H), 6.26 (d, 1H), 4.00 (m, 1H), |
| | | | | 3.41 (m, 1H), 3.21 (m, 1H), 2.92 (m, 1H), |
| | | | | 2.80 (m, 1H), 2.0 (m, 1H), 1.93 (m, 1H), |
| | | | | 1.76 (m, 1H), 1.69 (m, 1H). |
| 166 | 2-(3-fluorophenyl)-4- | 352.41 | 353 | 10.98 (s, 1H), 9.19 (m, 1H), 9.08 (m, |
| | (piperidin-3-ylamino)- | | | 1H), 7.63 (d, 2H), 7.56 (m, 2H), 7.50 (m, |
| | 1H-indole-7-carboxamide | | | 1H), 7.34 (s, 1H), 7.13 (dt, 1H), 6.26 (d, |
| | | | | 1H), 5.60 (br s, 2H), 4.00 (m, 1H), |
| | | | | 3.41 (m, 1H), 3.21 (m, 1H), 2.92 (m, 1H), |
| | | | | 2.80 (m, 1H), 2.0 (m, 1H), 1.93 (m, 1H), |
| | | | | 1.76 (m, 1H), 1.69 (m, 1H). |
| 167 | 2-(3-methoxyphenyl)-4- | 364.45 | 365 | 10.90 (s, 1H), 9.24 (m, 2H), 7.63 (d, |
| | (piperidin-3-ylamino)- | | | 2H), 7.39 (t, 2H), 7.29 (s, 2H), 7.26 (s, |
| | 1H-indole-7-carboxamide | | | 1H), 6.89 (dd, 2H), 6.26 (d, 1H), |
| | | | | 4.01 (m, 1H), 3.85 (s, 3H), 3.41 (m, 1H), |
| | | | | 3.23 (m, 1H), 2.92 (m, 1H), 2.80 (m, 1H), |
| | | | | 2.0 (m, 1H), 1.93 (m, 1H), 1.76 (m, 1H), |
| | | | | 1.69 (m, 1H). |
| 168 | 2-[3- | 377.49 | 378 | 10.97 (s, 1H), 9.26 (m, 2H), 7.95 (br s, |
| | (dimethylamino)phenyl]- | | | 2H), 7.49 (m, 3H), 7.33 (m, 2H), 6.26 (d, |
| | 4-(piperidin-3-ylamino)- | | | 1H), 4.03 (m, 1H), 3.68 (m, 1H), |
| | 1H-indole-7-carboxamide | | | 3.48 (m, 1H), 3.41 (m, 1H), 3.23 (m, 1H), |
| | | | | 3.12 (s, 6H), 2.92 (m, 1H), 2.80 (m, 1H), |
| | | | | 2.0 (m, 1H), 1.93 (m, 1H), 1.76 (m, 1H), |
| | | | | 1.69 (m, 1H). |
| 169 | 4-(piperidin-4-ylamino)- | 335.41 | 336 | 1.41 (td, 2H), 1.95 (d, 2H), 2.61 (t, 2H), |
| | 2-pyridin-4-yl-1H-indole- | | | 3.01 (d, 2H), 3.47-3.61 (m, 2H), |
| | 7-carboxamide | | | 6.16-6.21 (m, 2H), 7.52 (s, 1H), |
| | | | | 7.61-7.67 (m, 3H), 8.58 (d, 2H), 11.05 (s, 1H); |

The following examples 170-171 are prepared by chiral preparatory HPLC separation of example 160.

| | | | MS | |
|---|---|---|---|---|
| | | MW | (ES, | ¹H NMR (300 MHz; d₆-DMSO; δ ppm) |
| Example | IUPAC Name | (g/mol) | M + H) | unless otherwise noted |
| 170 | 2-phenyl-4-[(3R)- | 334.42 | 335 | 10.87 (s, 1H) 7.69 (d, 2H) 7.57 (d, 1H) |
| | piperidin-3-ylamino]- | | | 7.45 (t, 2H) 7.28 (t, 1H) 7.20 (s, 1H) |
| | 1H-indole-7- | | | 6.96 (br s, 1H) 6.15 (d, 1H) 6.02 (d, 1H) |
| | carboxamide | | | 3.50 (d, 1H) 3.30 (s, 2H) 3.05-3.20 (m, |
| | | | | 2H) 2.84 (d, 1H) 2.34-2.46 (m, 1H) |
| | | | | 1.98 (s, 1H) 1.60-1.72 (m, 1H) |
| | | | | 1.43-1.58 (m, 2H). |
| 171 | 2-phenyl-4-[(3S)- | 334.42 | 335 | 10.87 (s, 1H) 7.69 (d, 2H) 7.57 (d, 1H) |
| | piperidin-3-ylamino]- | | | 7.45 (t, 2H) 7.28 (t, 1H) 7.20 (s, 1H) |
| | 1H-indole-7- | | | 6.96 (br s, 1H) 6.15 (d, 1H) 6.02 (d, 1H) |
| | carboxamide | | | 3.50 (d, 1H) 3.30 (s, 2H) 3.05-3.20 (m, |
| | | | | 2H) 2.84 (d, 1H) 2.34-2.46 (m, 1H) |
| | | | | 1.98 (s, 1H) 1.60-1.72 (m, 1H) |
| | | | | 1.43-1.58 (m, 2H). |

The following examples 172-173 are prepared by chiral preparatory HPLC separation of example 166.

| | | | MS | |
|---|---|---|---|---|
| | | MW | (ES, | ¹H NMR (300 MHz; d₆-DMSO; δ ppm) |
| Example | IUPAC Name | (g/mol) | M + H) | unless otherwise noted |
| 172 | 2-(3-fluorophenyl)-4- | 352.41 | 353 | 10.98 (s, 1H), 9.19 (m, 1H), 9.08 (m, 1H), |
| | [(3S)-piperidin-3- | | | 7.63 (d, 2H), 7.56 (m, 2H), 7.50 (m, 1H), 7.34 (s, |
| | ylamino]-1H-indole- | | | 1H), 7.13 (dt, 1H), 6.26 (d, 1H), 5.60 (br s, 2H), |
| | 7-carboxamide | | | 4.00 (m, 1H), 3.41 (m, 1H), 3.21 (m, 1H), |
| | | | | 2.92 (m, 1H), 2.80 (m, 1H), 2.0 (m, 1H), 1.93 (m, |
| | | | | 1H), 1.76 (m, 1H), 1.69 (m, 1H). |
| 173 | 2-(3-fluorophenyl)-4- | 352.41 | 353 | 10.98 (s, 1H), 9.19 (m, 1H), 9.08 (m, 1H), |
| | [(3R)-piperidin-3- | | | 7.63 (d, 2H), 7.56 (m, 2H), 7.50 (m, 1H), |
| | ylamino]-1H-indole- | | | 7.34 (s, 1H), 7.13 (dt, 1H), 6.26 (d, 1H), |
| | 7-carboxamide | | | 5.60 (br s, 2H), 4.00 (m, 1H), 3.41 (m, 1H), |
| | | | | 3.21 (m, 1H), 2.92 (m, 1H), 2.80 (m, 1H), |
| | | | | 2.0 (m, 1H), 1.93 (m, 1H), 1.76 (m, 1H), |
| | | | | 1.69 (m, 1H). |

The following examples 174-175 are prepared by chiral preparatory HPLC separation of example 161.

| | | | MS | |
|---|---|---|---|---|
| | | MW | (ES, | ¹H NMR (300 MHz; d₆-DMSO; δ ppm) |
| Example | IUPAC Name | (g/mol) | M + H) | unless otherwise noted |
| 174 | 2-(4-chlorophenyl)-4- | 368.87 | 369 | 10.75 (s, 1H), 9.10 (m, 2H), 7.55 (d, 2H), |
| | [(3S)-piperidin-3- | | | 7.45 (d, 1H), 7.33 (d, 2H), 7.15 (d, 1H), |
| | ylamino]-1H-indole- | | | 6.08 (d, 1H), 5.45 (br s, 3H), 3.83 (m, 1H), |
| | 7-carboxamide | | | 3.21 (m, 1H), 3.03 (m, 1H), 2.73 (m, 1H), |
| | | | | 2.65 (m, 1H), 1.80 (m, 2H), 1.62 (m, 1H), |
| | | | | 1.51 (m, 1H). |
| 175 | 2-(4-chlorophenyl)-4- | 368.87 | 369 | 10.75 (s, 1H), 9.10 (m, 2H), 7.55 (d, 2H), |
| | [(3R)-piperidin-3- | | | 7.45 (d, 1H), 7.33 (d, 2H), 7.15 (d, 1H), |
| | ylamino]-1H-indole- | | | 6.08 (d, 1H), 5.45 (br s, 3H), 3.83 (m, 1H), |
| | 7-carboxamide | | | 3.21 (m, 1H), 3.03 (m, 1H), 2.73 (m, 1H), |
| | | | | 2.65 (m, 1H), 1.80 (m, 2H), 1.62 (m, 1H), |
| | | | | 1.51 (m, 1H). |

The following examples 176-177 are prepared by chiral preparatory HPLC separation of example 162.

| | | | MS | |
|---|---|---|---|---|
| | | MW | (ES, | ¹H NMR (300 MHz; d₆-DMSO; δ ppm) |
| Example | IUPAC Name | (g/mol) | M + H) | unless otherwise noted |
| 176 | 2-(4-fluorophenyl)-4- | 352.41 | 353 | 10.89 (s, 1H), 8.92 (m, 1H), 8.73 (m, 1H), |
| | [(3S)-piperidin-3- | | | 7.75 (d, 1H), 7.74 (d, 1H), 7.60 (d, 1H), |
| | ylamino]-1H-indole- | | | 7.30 (t, 2H), 7.15 (d, 1H), 7.04 (br s, 1H), |
| | 7-carboxamide | | | 6.33 (br s, 1H), 6.23 (d, 1H), 3.91 (m, 1H), |
| | | | | 3.41 (m, 1H), 3.25 (m, 1H), 2.89 (m, 1H), |
| | | | | 2.75 (m, 1H), 2.03 (m, 1H), 1.94 (m, 1H), |
| | | | | 1.74 (m, 1H), 1.66 (m, 1H). |
| 177 | 2-(4-fluorophenyl)-4- | 352.41 | 353 | 10.89 (s, 1H), 8.92 (m, 1H), 8.73 (m, 1H), |
| | [(3R)-piperidin-3- | | | 7.75 (d, 1H), 7.74 (d, 1H), 7.60 (d, 1H), |
| | ylamino]-1H-indole- | | | 7.30 (t, 2H), 7.15 (d, 1H), 7.04 (br s, 1H), |
| | 7-carboxamide | | | 6.33 (br s, 1H), 6.23 (d, 1H), 3.91 (m, 1H), |
| | | | | 3.41 (m, 1H), 3.25 (m, 1H), 2.89 (m, 1H), |
| | | | | 2.75 (m, 1H), 2.03 (m, 1H), 1.94 (m, 1H), |
| | | | | 1.74 (m, 1H), 1.66 (m, 1H). |

### Example 178

### N-methyl-2-phenyl-4-[(3S)-piperidin-3-ylamino]thieno[3,2-c]pyridine-7-carboxamide

4-{[(3S)-1-(tert-butoxycarbonyl)piperidin-3-yl]amino}-2-phenylthieno[3,2-c]pyridine-7-carboxylic acid. To tert-butyl (3S)-3-[(7-cyano-2-phenylthieno[3,2-c]pyridin-4-yl)amino]piperidine-1-carboxylate (2.00 grams, 4.60 mmol) is added 6N HCl (50 mL) and the resulting solution is heated to reflux overnight or until LCMS indicated complete conversion to product. The reaction mixture is then cooled to rt, concentrated under reduced pressure and dried in a vacuum oven for 24 hours to afford the title compound. LCMS (ES, M+H=354).

tert-butyl (3S)-3-({7-[(methylamino)carbonyl]-2-phenylthieno[3,2-c]pyridin-4-yl)amino}piperidine-1-carboxylate. 4-{[(3S)-1-(tert-butoxycarbonyl)piperidin-3-yl]amino}-2-phenylthieno[3,2-c]pyridine-7-carboxylic acid is added to a round bottom flask containing HATU (81.0 mg, 0.213 mmol), methylamine (2M in THF, 0.200 mL, 0.426 mmol), DIPEA (0.037 mL, 0.213 mmol), and DMF (1.0 mL). The reaction is stirred at rt for 12 hours whereupon the reaction mixture is washed with saturated NH₄Cl solution (2×20 mL) and extracted with EtOAc (2×20 mL). The organic layers are combined, dried over magnesium sulfate, filtered, and concentrated under reduced pressure. The mixture is purified using MPLC (SiO₂; 100% hexanes to 100% EtOAc) to yield the title compound. LCMS (ES, M+H=467).

N-methyl-2-phenyl-4-[(3S)-piperidin-3-ylamino]thieno[3,2-c]pyridine-7-carboxamide. To tert-butyl (3S)-3-({7-[(methylamino)carbonyl]-2-phenylthieno[3,2-c]pyridin-4-yl}amino)piperidine-1-carboxylate is added 4N HCl in dioxane solution (5.0 mL) and the reaction is stirred at rt for 20 minutes whereupon the reaction is concentrated under reduced pressure to yield the title compound. ¹H NMR δ 8.93 (m, 1H), 8.77 (m, 1H), 8.49 (m, 2H), 8.28 (m, 1H), 7.73 (d, 2H), 7.50 (m, 2H), 7.38 (m, 1H), 4.54 (m, 1H), 3.21 (m, 2H), 2.92 (m, 2H), 2.83 (d, 3H), 2.00 (m, 2H), 1.72 (m, 2H). LCMS (ES, M+H=367).

Example 179 is synthesized in an analogous fashion.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 179 | 2-phenyl-4-[(3S)- | 429.55 | 430 | 11.57 (s, 1H), 9.03 (s, 1H), 8.72 (d, |
| | piperidin-3-ylamino]-N- | | | 1H), 8.43 (s, 1H), 8.36 (d, 2H), |
| | pyridin-4-ylthieno[3,2- | | | 8.10 (m, 1H), 7.76 (m, 2H), 7.68 (m, 1H), |
| | c]pyridine-7- | | | 7.62 (s, 1H), 7.52 (m, 2H), 7.41 (m, |
| | carboxamide | | | 1H), 4.63 (m, 1H), 3.21 (m, 2H), |
| | | | | 2.98 (m, 2H), 2.00 (m, 2H), 1.76 (m, 2H) |

### Example 180

### 2-phenyl-4-[(3S)-piperidin-3-ylamino]-N-pyrazin-2-ylthieno[3,2-c]pyridine-7-carboxamide

4-{[(3S)-1-(tert-butoxycarbonyl)piperidin-3-yl]amino}-2-phenylthieno[3,2-c]pyridine-7-carboxylic acid. To tert-butyl (3S)-3-[(7-cyano-2-phenylthieno[3,2-c]pyridin-4-yl)amino]piperidine-1-carboxylate (2.00 grams, 4.60 mmol) is added 6N HCl (50 mL) and the resulting solution is heated to reflux overnight or until LCMS indicated complete conversion to product. The reaction is then cooled to rt, concentrated under reduced pressure and dried in a vacuum oven for 24 hours to afford the title compound. LCMS (ES, M+H=354).

tert-butyl (3S)-3-([2-phenyl-7-[(pyrazin-2-ylamino)carbonyl]thieno[3,2-c]pyridin-4-yl]amino)piperidine-1-carboxylate. To a round bottom flask containing aminopyrazine (113 mg, 1.19 mmol) in toluene (1.0 mL) is added at 0° C. trimethylaluminum (2.0 M in hexanes, 0.600 mL, 1.19 mmol). The solution is stirred for thirty minutes at rt and then added to a round bottom flask containing 4-{[(3S)-1-(tert-butoxycarbonyl)piperidin-3-yl]amino}-2-phenylthieno[3,2-c]pyridine-7-carboxylic acid (108 mg, 0.238 mmol), HATU (136 mg, 0.358 mmol), DIPEA (0.064 mL, 0.358 mmol), and DMF (1.0 mL). The reaction is stirred at 100° C. for 12 hours whereupon the reaction mixture is washed with saturated NH₄Cl solution (2×20 mL) and extracted with EtOAc (2×20 mL). The organic layers are combined, dried over magnesium sulfate, filtered, and concentrated under reduced pressure. The mixture is purified by preparatory HPLC (5% to 95% MeCN/water/0.1% TFA) to yield the title compound. LCMS (ES, M+H=531).

2-phenyl-4-[(3S)-piperidin-3-ylamino]-N-pyrazin-2-ylthieno[3,2-c]pyridine-7-carboxamide. To tert-butyl (3S)-3-({2-phenyl-7-[(pyrazin-2-ylamino)carbonyl]thieno[3,2-c]pyridin-4-yl}amino)piperidine-1-carboxylate is added 4N HCl in dioxane solution (5.0 mL) and the reaction is stirred at rt for 20 minutes whereupon the reaction is concentrated under reduced pressure to yield the title compound. ¹H NMR δ 11.12 (s, 1H), 9.44 (s, 1H), 8.94 (s, 1H), 8.80 (br s, 1H), 8.48 (s, 1H), 8.42 (s, 1H), 8.34 (s, 1H), 7.88 (m, 1H), 7.76 (m, 2H), 7.52 (m, 2H), 7.40 (m, 1H), 4.61 (m, 1H), 3.24 (m, 2H), 2.94 (m, 2H), 2.02 (m, 2H), 1.75 (m, 2H). LCMS (ES, M+H=431).

### Example 181

### 4-{[2-(hydroxymethyl)piperidin-3-yl]oxy}-2-phenylthieno[3,2-c]pyridine-7-carboxamide

2-({[tert-butyl(dimethyl)silyl]oxy}methyl)pyridin-3-ol. To 2-(hydroxymethyl)pyridin-3-ol (14.86 g, 91.96 mmol) in 150 mL THF is added tert-butyldimethylsilylchloride (15.2 g, 101 mmol) and N,N,-dimethylaminopyridine (20.0 g, 101 mmol). The reaction is stirred at rt for four hours whereupon the reaction is extracted with EtOAc (3×100 mL) and washed with water. The combined organic layers are dried over magnesium sulfate, filtered, and concentrated under reduced pressure to afford after MPLC purification (SiO₂; 100% hexanes to 100% EtOAc) the title compound as a white solid. LCMS (ES, M+H=240).

2-({[tert-butyl(dimethyl)silyl]oxy}methyl)piperidin-3-ol. To a high pressure vessel containing 2-({[tert-butyl(dimethyl)silyl]oxy}methyl)pyridin-3-ol (4.00 g, 16.7 mmol) is added 10 mL each of EtOH and water followed by platinum (IV) oxide (1.00 g) under nitrogen. The high-pressure vessel is evacuated under reduced pressure and placed on a Parr hydrogenation apparatus at 50 psi for 24 hours. The mixture is then evacuated under nitrogen, filtered over a bed of diatomaceous earth, and rinsed with copious amounts of MeOH. The collected filtrate is concentrated in vacuo to afford the title compound as a mixture of isomers (approximately 10% of a minor diastereomer). LCMS (ES, M+H=246).

2-bromo-4-{[2-({[tert-butyl(dimethyl)silyl]oxy}methyl)piperidin-3-yl]oxy}thieno[3,2-c]pyridine-7-carbonitrile. To the 2-({[tert-butyl(dimethyl)silyl]oxy}methyl)piperidin-3-ol (295 mg, 1.20 mmol) dissolved in 3.0 mL THF is added sodium hydride (30.0 mg, 1.20 mmol) and the resulting mixture is stirred for 20 minutes at rt. A slurry of 2-bromo-4-chlorothieno[3,2-c]pyridine-7-carbonitrile (293 mg, 1.07 mmol) in 3.0 μL THF is then added and the reaction stirred at rt for one hour. The resulting mixture is diluted with sodium bicarbonate (10 mL) and extracted with EtOAc (2×20 mL). The organic layers are combined, dried over magnesium sulfate, filtered, and concentrated under reduced pressure to yield the title compound. LCMS (ES, M+H=483).

4-{[2-({[tert-butyl(dimethyl)silyl]oxy}methyl)piperidin-3-yl]oxy}-2-phenylthieno[3,2-c]pyridine-7-carbonitrile. To the 2-bromo-4-{[2-({[tert-butyl(dimethyl)silyl]oxy}methyl)piperidin-3-yl]oxy}thieno[3,2-c]pyridine-7-carbonitrile (516 mg, 1.07 mmol) is added phenylboronic acid (194 mg, 1.61 mmol), cesium carbonate (1.04 g, 3.21 mmol), dioxane/water (4 mL/2 mL) and then Pd(PPh₃)₄ (124 mg, 0.107 mmol). The reaction is heated to 80° C. for one hour whereupon the reaction is cooled to rt, filtered, and purified using MPLC (SiO₂; 100% hexanes to 100% EtOAc) to afford the title compound. LCMS (ES, M+H=480).

4-{[2-({[tert-butyl(dimethyl)silyl]oxy}methyl)piperidin-3-yl]oxy}-2-phenylthieno[3,2-c]pyridine-7-carboxamide. To a flask containing 4-{[2-({[tert-butyl(dimethyl)silyl]oxy}methyl)piperidin-3-yl]oxy}-2-phenylthieno[3,2-c]pyridine-7-carbonitrile is added 5.00 mL of 12 N HCl. The reaction mixture is stirred at rt and monitored by LCMS. Additional 12 N HCl is added every twelve hours to afford complete conversion to the desired product. Upon completion, the reaction mixture is diluted with MeOH and concentrated under reduced pressure to yield the product, which is purified by MPLC (SiO₂; 100% CH₂Cl₂ to 20% MeOH/CH₂Cl₂/3% NH₄OH) to afford the title compound as a mixture of isomers (approximately 10% minor diastereomer). Analytical data provided for major isomer present in mixture: ¹H NMR δ 9.31 (m, 1H), 8.93 (m, 1H), 8.64 (s, 1H), 8.44 (s, 1H), 8.27 (m, 1H), 7.84 (d, 2H), 7.69 (m, 1H), 7.46 (m, 3H), 5.70 (m, 1H), 3.68 (m, 2H), 3.54 (m, 1H), 3.34 (m, 1H), 3.06 (m, 1H), 2.17 (m, 1H), 1.85 (m, 2H), 1.67 (m, 1H). LCMS (ES, M+H=384).

### Example 182

### 4-{[2-(hydroxymethyl)piperidin-3-yl]amino}-2-phenylthieno[3,2-c]pyridine-7-carboxamide

2-(methoxycarbonyl)nicotinic acid. To furo[3,4-b]pyridine-5,7-dione (41.0 g, 275 mmol) is added 200 mL of MeOH. The reaction is heated to reflux for approximately one hour followed by concentration in vacuo to afford the title compound and 3-(methoxycarbonyl)pyridine-2-carboxylic acid (2.3:1, respectively) as a mixture of isomers. LCMS (ES, M+H=182).

methyl 3-[(tert-butoxycarbonyl)amino]pyridine-2-carboxylate. To a mixture of 2-(methoxycarbonyl)nicotinic acid and 3-(methoxycarbonyl)pyridine-2-carboxylic acid (10.46 g, 57.7 mmol) is added tert-butanol (100 mL) and TEA (8.85 mL, 63.5 mmol). The reaction is stirred for five minutes at rt and then diphenyl phosphoryl azide (13.1 mL, 60.6 mmol) is added. The reaction is heated to reflux and stirred for approximately four hours. The reaction mixture is cooled to rt, concentrated to dryness, re-dissolved in EtOAc, and washed with water and saturated sodium bicarbonate (2×20 mL each). The organic layers are combined, dried over magnesium sulfate, filtered, and concentrated under reduced pressure. The mixture is purified by column chromatography (100% hexanes to 100% EtOAc) to yield the title compound and methyl 2-[(tert-butoxycarbonyl)amino]nicotinate (9.24 g, 64% yield). LCMS (ES, M+Na=275).

tert-butyl [2-(hydroxymethyl)pyridin-3-yl]carbamate. To methyl 3-[(tert-butoxycarbonyl)amino]pyridine-2-carboxylate and methyl 2-[(tert-butoxycarbonyl)amino]nicotinate (5.00 g, 19.8 mmol) is added THF/MeOH (30 mL/3 mL) and the reaction is cooled to 0° C. whereupon sodiumborohydride (1.49 g, 39.6 mmol) is added. The reaction is warmed to rt and stirred for four hours. The reaction mixture is then dissolved in EtOAc and washed with saturated sodium bicarbonate solution. The organic layers are combined, dried over magnesium sulfate, filtered, and concentrated under reduced pressure to afford the title compound and tert-butyl [3-(hydroxymethyl)pyridin-2-yl]carbamate which are separated by preparatory HPLC (5-95% MeCN/water/0.1% TFA). The desired isomer is confirmed by 1D NOE NMR experiments. ¹H NMR δ 8.78 (br s, 1H), 8.17 (m, 1H), 8.10 (d, 1H), 7.27 (dd, 1H), 4.64 (s, 2H), 1.46 (s, 9H). LCMS (ES, M+H=225).

tert-butyl [2-(hydroxymethyl)piperidin-3-yl]carbamate. To a high pressure vessel containing tert-butyl [2-(hydroxymethyl)pyridin-3-yl]carbamate (1.46 g, 6.51 mmol) is added 5 mL each of EtOH and water followed by platinum (IV) oxide (500 mg) under nitrogen. The high pressure vessel is evacuated under reduced pressure and placed on a Parr hydrogenation apparatus at 50 psi for 24 hours. The mixture is then evacuated under nitrogen, filtered over a bed of diatomaceous earth, and rinsed with copious amounts of MeOH. The collected filtrate is concentrated in vacuo to afford the title compound as a mixture of isomers. MS m/z 231 (M+H).

benzyl 3-amino-2-(hydroxymethyl)piperidine-1-carboxylate. To a round bottom flask is added tert-butyl [2-(hydroxymethyl)piperidin-3-yl]carbamate (785 mg, 3.41 mmol), DIPEA (0.653 mL, 3.75 mmol), and CH₂Cl₂ (10 mL). The flask is cooled to 0° C. and benzyl chloridocarbonate (0.504 mL, 3.58 mmol) is added. The reaction is warmed to rt and stirred for 12 hours whereupon the mixture is extracted with CH₂Cl₂ and EtOAc and washed with saturated sodium bicarbonate. The combined organic layers are dried over magnesium sulfate, filtered, and concentrated under reduced pressure. The residue is purified by MPLC (SiO₂; 100% hexanes to 100% EtOAc to 20% MeOH/CH₂Cl₂) and treated directly with a 4N HCl in dioxane solution (5 mL) for thirty minutes. The reaction mixture is concentrated under reduced pressure to afford the title compound. LCMS (ES, M+H=265).

benzyl 3-[(2-bromo-7-cyanothieno[3,2-c]pyridin-4-yl)amino]-2-(hydroxymethyl)piperidine-1-carboxylate. To a round bottom flask containing benzyl 3-amino-2-(hydroxymethyl)piperidine-1-carboxylate (246 mg, 0.932 mmol) is added 2-bromo-4-chlorothieno[3,2-c]pyridine-7-carbonitrile (128 mg, 0.466 mmol), potassium carbonate (100 mg, 0.700 mmol) and NMP (5.0 mL). The reaction mixture is heated to 80° C. and monitored by LCMS every hour for completion whereupon the mixture is cooled to rt. Water is added (50 mL) and the resulting solid is filtered and dried under reduced pressure for 12 hours to yield the title compound. LCMS (ES, M+H=502).

benzyl 3-[(7-cyano-2-phenylthieno[3,2-c]pyridin-4-yl)amino]-2-(hydroxymethyl)piperidine-1-carboxylate. To the benzyl 3-[(2-bromo-7-cyanothieno[3,2-c]pyridin-4-yl)amino]-2-(hydroxymethyl)piperidine-1-carboxylate (0.466 mmol) is added phenylboronic acid (0.699 mmol), cesium carbonate (0.932 mmol), dioxane/water (2.0 mL/1.0 mL) and then Pd(PPh₃)₄ (0.0466 mmol). The reaction is heated to 80° C. for one hour whereupon the reaction is cooled to rt, filtered, and purified using MPLC (SiO₂; 100% hexanes to 100% EtOAc) to afford the title compound. LCMS (ES, M+H=499).

4-{[2-(hydroxymethyl)piperidin-3-yl]amino}-2-phenylthieno[3,2-c]pyridine-7-carboxamide. To a flask containing benzyl 3-[(7-cyano-2-phenylthieno[3,2-c]pyridin-4-yl)amino]-2-(hydroxymethyl)piperidine-1-carboxylate is added 5.00 mL of 12 N HCl. The reaction mixture is stirred at rt and monitored by LCMS. Additional 12 N HCl is added every twelve hours to afford complete conversion to the desired product. Upon completion, the reaction mixture is diluted with MeOH and concentrated under reduced pressure to yield product, which is purified by preparatory HPLC (5-95% MeCN/water/0.1% TFA) to afford the title compound as a mixture of isomers (in an approximate 1/1 ratio). ¹H NMR δ 9.95 (m, 1H), 9.21 (m, 1H), 8.98 (m, 1H), 8.70 (m, 1H), 8.51 (m, 1H), 8.38 (m, 1H), 8.15 (m, 1H), 7.77 (m, 2H), 7.44 (m, 3H), 4.88 (m, 1H), 3.76 (m, 1H), 3.28 (m, 2H), 2.96 (m, 2H), 2.10 (m, 2H), 1.87 (m, 2H). LCMS (ES, M+H=383).

### Example 183

### 2-phenyl-7-[(3S)-piperidin-3-yloxy]-1H-benzimidazole-4-carboxamide

4-fluoro-3-nitrobenzamide. To 4-fluoro-3-nitrobenzoic acid (12.0 g, 64.8 mmol) is added CH₂Cl₂ (300 mL), oxalyl chloride (16.7 mL, 195 mmol) and approximately 0.100 mL DMF drop wise. The mixture is stirred at rt for three hours whereupon the reaction is cooled to −78° C. and liquid NH₃ is bubbled through the solution for approximately twenty minutes. The resulting yellow solid is purified by MPLC (SiO₂; 100% hexanes to 100% EtOAc) to afford the title compound (9.00 g, 76% yield). LCMS (ES, M−H=183).

tert-butyl (3S)-3-[4-(aminocarbonyl)-2-nitrophenoxy]piperidine-1-carboxylate. To a solution containing tert-butyl (3S)-3-hydroxypiperidine-1-carboxylate (3.97 g, 19.7 mmol) dissolved in 5.00 mL of DMF is added sodium hydride (473 mg, 19.7 mmol). The resulting solution is stirred at rt for thirty minutes followed by the addition of 4-fluoro-3-nitrobenzoic acid (3.29 g, 17.9 mmol) dissolved in 5.00 mL DMF. The mixture is stirred for twelve hours at rt or until LCMS indicates complete conversion to product. To the reaction mixture is then added 20 mL water and the resulting solid is filtered and dried under reduced pressure to afford the title compound (4.15 g, 63% yield). LCMS (ES, M+H=366).

tert-butyl (3S)-3-[2-amino-4-(aminocarbonyl)phenoxy]piperidine-1-carboxylate. To solution of tert-butyl (3S)-3-[4-(aminocarbonyl)-2-nitrophenoxy]piperidine-1-carboxylate (4.15 g, 11.4 mmol) dissolved in 50 mL MeOH is added 10% Pd/C (800 mg). The resulting mixture is charged with hydrogen for twelve hours or until LCMS indicates complete conversion to product. The mixture is filtered over diatomaceous earth and rinsed with copious amounts of MeOH to yield the desired product after purification by MPLC (SiO₂; 100% hexanes to 100% EtOAc to 20% MeOH/CH₂Cl₂). LCMS (ES, M+H=336).

tert-butyl(3S)-3-(4-(aminocarbonyl)-2{[imino(phenyl)methyl]amino}phenoxy)piperidine-1-carboxylate. Trimethyl aluminum (2M in hexanes, 17.9 mL, 35.8 mmol) is added at 0° C. to a solution of tert-butyl (3S)-3-[2-amino-4-(aminocarbonyl)phenoxy]piperidine-1-carboxylate (1.20 g, 3.58 mmol) in 20 mL THF. The mixture is warmed to rt and stirred for one hour whereupon a solution of benzonitrile (3.66 mL, 35.8 mmol) in 10 mL THF is added. The solution is stirred at 60° C. until LCMS indicates complete consumption of starting material. The mixture is cooled to 0° C. A 10% Rochelle's salt solution is added drop wise (approximately 20 mL). The mixture is extracted with EtOAc (4×20 mL), organic layers are dried over magnesium sulfate, filtered, and concentrated in vacuo to afford material which is purified on MPLC (SiO₂; CH₂Cl₂ to 20% MeOH/CH₂Cl₂) to afford the title compound (464 mg, 30% yield). LCMS (ES, M+H=439).

tert-butyl(3S)-3-{[4-(aminocarbonyl)-2-phenyl-1H-benzimidazol-7-yl]oxy}piperidine-1-carboxylate. To a solution of tert-butyl (3S)-3-(4-(aminocarbonyl)-2-{[imino(phenyl)methyl]amino}phenoxy)piperidine-1-carboxylate dissolved in 3.0 mL each of MeOH and water is added sodium hypochlorite (0.100 mL, 1.17 mmol) drop wise. The resulting solution is stirred at rt for five minutes whereupon sodium carbonate (148 mg) is added in 3.0 mL of water. The solution is then heated to reflux and monitored by LCMS for completion. Upon consumption of the starting material, the mixture is cooled to rt, extracted with EtOAc and CH₂Cl₂/MeOH (1/1), and the organic layers are dried over magnesium sulfate, filtered, and concentrated under reduced pressure. The resulting mixture is purified by MPLC (SiO₂; 100% CH₂Cl₂ to 20% MeOH/CH₂Cl₂) to afford the title compound. LCMS (ES, M+H=437).

2-phenyl-7-[(3S)-piperidin-3-yloxy]-1H-benzimidazole-4-carboxamide. To tert-butyl (3S)-3-{[4-(aminocarbonyl)-2-phenyl-1H-benzimidazol-7-yl]oxy}piperidine-1-carboxylate (18.7 mg, 0.043 mmol) is added 5.0 mL of 4.0 N HCl in dioxane solution. The reaction is stirred at rt for approximately thirty minutes, concentrated in vacuo under reduced pressure, and dried under high vacuum to afford the title compound. ¹H NMR δ 9.60 (m, 1H), 9.10 (m, 2H), 8.48 (s, 2H), 7.98 (m, 1H), 7.85 (m, 1H), 7.63 (m, 3H), 7.03 (d, 1H), 5.16 (m, 1H), 4.13 (m, 1H), 3.69 (m, 1H), 3.46 (m, 1H), 3.27 (m, 1H), 2.00 (m, 2H), 1.79 (m, 1H), 1.62 (m, 1H). LCMS (ES, M+H=337).

### Example 184

### 2-{4-[4-(methylsulfonyl)piperazin-1-yl]phenyl}-4-[(3S)-piperidin-3-ylamino]thieno[3,2-c]pyridine-7-carboxamide

1-[4-(4,4,5,5-tetramethyl-1,3,2-dioxaborolan-2-yl)phenyl]piperazine. To tert-butyl 4-[4-(4,4,5,5-tetramethyl-1,3,2-dioxaborolan-2-yl)phenyl]piperazine-1-carboxylate (235 mg, 0.656 mmol) is added 5.0 mL of 4N HCl in dioxane and the resulting solution is stirred at rt for two hours whereupon the solution is concentrated under reduced pressure to afford the title compound as a white solid. ¹H NMR δ 9.14 (br s, 1H), 7.54 (d, 2H), 6.96 (d, 2H), 3.43 (m, 4H), 3.18 (m, 4H), 1.25 (s, 12H).

tert-butyl (3S)-3-{[7-cyano-2-(4-piperazin-1-ylphenyl)thieno[3,2-c]pyridin-4-yl]amino}piperidine-1-carboxylate. To 1-[4-(4,4,5,5-tetramethyl-1,3,2-dioxaborolan-2-yl)phenyl]piperazine (58.0 mg, 0.225 mmol) is added tert-butyl (3S)-3-[(2-bromo-7-cyanothieno[3,2-c]pyridin-4-yl)amino]piperidine-1-carboxylate (361 mg, 0.826 mmol), cesium carbonate (806 mg, 2.48 mmol), Pd(PPh₃)₄ (95.4 mg, 0.0826 mmol) and dioxane/water (2.0 mL/1.0 mL). The reaction is stirred at 80° C. for thirty minutes, cooled to rt, filtered, rinsed with copious amounts of EtOAc, dried over magnesium sulfate, and concentrated under reduced pressure. The mixture is purified using MPLC (SiO₂; 100% CH₂Cl₂ to 20% CH₃OH/CH₂Cl₂/3% NH₄OH) to afford the title compound. LCMS (ES, M+H=519).

tert-butyl (3S)-3-[(7-cyano-2-{4-[4-(methylsulfonyl)piperazin-1-yl]phenyl}thieno[3,2-c]pyridin-4-yl)amino]piperidine-1-carboxylate. To tert-butyl (3S)-3-{[7-cyano-2-(4-piperazin-1-ylphenyl)thieno[3,2-c]pyridin-4-yl]amino}piperidine-1-carboxylate (74.0 mg, 0.143 mmol) dissolved in 5.0 mL THF is added TEA (0.0239 mL, 0.1721 mmol) and then methanesulfonylchloride (0.0133 mL, 0.172 mmol) drop wise. The reaction is stirred at rt for approximately one hour whereupon the reaction mixture is washed with a saturated sodium bicarbonate solution (2×20 mL) and extracted with EtOAc (2×20 mL). The organic layers are dried over magnesium sulfate, filtered, and concentrated under reduced pressure to afford the title compound after purification using MPLC (SiO₂; 100% CH₂Cl₂ to 20% CH₃OH/CH₂Cl₂/3% NH₄OH). LCMS (ES, M+H=597).

2-{4-[4-(methylsulfonyl)piperazin-1-yl]phenyl}-4-[(3S)-piperidin-3-ylamino]thieno[3,2-c]pyridine-7-carboxamide. To tert-butyl (3S)-3-[(7-cyano-2-{4-[4-(methylsulfonyl)piperazin-1-yl]phenyl}thieno[3,2-c]pyridin-4-yl)amino]piperidine-1-carboxylate is added approximately 5 mL 12N HCl and the resulting solution is stirred at rt for 12 hours or until LCMS indicates complete conversion to the desired product. The resulting reaction mixture is diluted with MeOH, concentrated under reduced pressure and purified using MPLC (SiO₂; 100% CH₂Cl₂ to 20% CH₃OH/CH₂Cl₂/3% NH₄OH) to yield the title compound. ¹H NMR δ 9.40 (m, 1H), 8.87 (m, 1H), 8.48 (s, 2H), 8.31 (m, 2H), 7.66 (d, 2H), 7.12 (d, 2H), 4.53 (m, 1H), 3.38 (m, 5H), 3.25 (m, 5H), 3.03 (m, 2H), 2.94 (s, 3H), 2.01 (m, 2H), 1.78 (m, 2H). LCMS (ES, M+H=515).

Examples 185-189 are prepared in a similar fashion using the appropriate starting materials.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 185 | 2-[4-(4-acetylpiperazin-1- | 478.62 | 479 | 9.23 (m, 1H), 8.84 (m, 1H), 8.47 (s, |
| | yl)phenyl]-4-[(3S)- | | | 1H), 8.26 (m, 1H), 7.62 (d, 2H), |
| | piperidin-3- | | | 7.08 (d, 2H), 4.51 (m, 1H), 3.58 (m, 5H), |
| | ylamino]thieno[3,2- | | | 3.45 (m, 2H), 3.24 (m, 5H), |
| | c]pyridine-7-carboxamide | | | 2.04 (overlapping s and m, 5H), 1.80 (m, |
| | | | | 2H) |
| 186 | 2-{3-[4- | 514.67 | 515 | 9.56 (m, 1H), 8.94 (m, 2H), 8.51 (s, |
| | (methylsulfonyl)piperazin- | | | 1H), 8.42 (m, 1H), 7.69 (m, 1H), |
| | 1-yl]phenyl}-4-[(3S)- | | | 7.39 (m, 2H), 7.24 (m, 1H), 7.06 (m, |
| | piperidin-3- | | | 1H), 4.59 (m, 1H), 3.50 (m, 1H), |
| | ylamino]thieno[3,2- | | | 3.32 (m, 8H), 3.08 (m, 2H), 2.95 (s, |
| | c]pyridine-7-carboxamide | | | 3H), 2.74 (m, 1H), 1.79 (m, 4H) |
| 187 | 2-[3-(4-acetylpiperazin-1- | 478.62 | 479 | 9.44 (m, 1H), 8.92 (m, 1H), 8.72 (m, |
| | yl)phenyl]-4-[(3S)- | | | 1H), 8.51 (s, 1H), 8.32 (m, 1H), |
| | piperidin-3- | | | 7.63 (m, 1H), 7.36 (m, 2H), 7.22 (d, 1H), |
| | ylamino]thieno[3,2- | | | 7.04 (m, 1H), 4.56 (m, 1H), 3.61 (m, |
| | c]pyridine-7-carboxamide | | | 4H), 3.46 (m, 2H), 3.24 (m, 5H), |
| | | | | 3.01 (m, 1H), 2.01 (overlapping s |
| | | | | and m, 5H), 1.78 (m, 2H) |
| 188 | 2-(4-piperazin-1-ylphenyl)- | 436.58 | 437 | 9.82 (m, 1H), 9.36 (m, 1H), 9.08 (m, |
| | 4-[(3S)-piperidin-3- | | | 1H), 8.91 (m, 1H), 8.57 (m, 1H), |
| | ylamino]thieno[3,2- | | | 8.49 (s, 1H), 7.76 (m, 1H), 7.69 (d, |
| | c]pyridine-7-carboxamide | | | 2H), 7.11 (d, 2H), 4.64 (m, 1H), |
| | | | | 3.49 (m, 5H),. 3.28 (m, 6H), 3.02 (m, 1H), |
| | | | | 2.05 (m, 2H), 1.83 (m, 2H) |
| 189 | 2-(3-piperazin-1-ylphenyl)- | 436.58 | 437 | 9.65 (m, 1H), 9.21 (m, 1H), 9.08 (m, |
| | 4-[(3S)-piperidin-3- | | | 1H), 8.94 (m, 1H), 8.53 (s, 1H), |
| | ylamino]thieno[3,2- | | | 8.38 (m, 1H), 7.64 (m, 1H), 7.38 (m, 2H), |
| | c]pyridine-7-carboxamide | | | 7.22 (m, 2H), 4.64 (m, 1H), 3.49 (m, |
| | | | | 5H), 3.23 (m, 6H), 3.02 (m, 1H), |
| | | | | 2.02 (m, 2H), 1.80 (m, 2H) |

### Example 190

### 4-[(4-hydroxypiperidin-3-yl)amino]-2-phenylthieno[3,2-c]pyridine-7-carboxamide

Prepared in a similar fashion to Example 1 but using benzyl trans-3-amino-4-hydroxypiperidine-1-carboxylate (synthesis described in J. Med. Chem. 1997, 40, 226) as the starting material in step 7. ¹H NMR δ 9.29 (m, 1H), 8.85 (m, 1H), 8.60 (m, 1H), 8.51 (s, 1H), 8.19 (m, 1H), 7.77 (d, 2H), 7.56 (m, 1H), 7.50 (dd, 2H), 7.40 (dd, 1H), 4.42 (m, 1H), 4.0-4.3 (br s, 1H), 3.88 (m, 1H), 3.51 (m, 1H), 3.27 (m, 1H), 3.05 (m, 2H), 2.16 (m, 1H), 1.73 (m, 1H). LCMS (ES, M+H=369).

Example 191 is made in a similar fashion.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 191 | 4-[(4-hydroxypiperidin- | 374.49 | 375 | 9.07 (m, 1H), 8.80 (m, 1H), 8.50 (s, 1H), |
| | 3-yl)amino]-2-(3- | | | 8.28 (m, 1H), 8.10 (m, 1H), 7.85 (m, |
| | thienyl)thieno[3,2- | | | 1H), 7.73 (dd, 1H), 7.48 (dd, 1H), |
| | c]pyridine-7- | | | 7.44 (m, 1H), 4.39 (m, 1H), 3.8-4.2 (br s, |
| | carboxamide | | | 1H), 3.8 (m, 1H), 3.49 (m, 1H), 3.27 (m, |
| | | | | 1H), 3.01 (m, 2H), 2.14 (m, 1H), |
| | | | | 1.71 (m, 1H) |

### Example 192

### 4-[(3-hydroxypiperidin-4-yl)amino]-2-phenylthieno[3,2-c]pyridine-7-carboxamide

Prepared in a similar fashion to Example 1 but using benzyl trans-4-amino-3-hydroxypiperidine-1-carboxylate (synthesis described in J. Med. Chem. 1997, 40, 226) as the starting material in step 7. ¹H NMR δ 8.85 (m, 1H), 8.73 (m, 1H), 8.48 (s, 1H), 8.36 (br, 1H), 8.08 (m, 1H), 7.75 (d, 2H), 7.55 (br, 1H), 7.51 (t, 2H), 7.40 (t, 1H), 5.74 (br, 1H), 4.33 (m, 1H), 3.93 (m, 1H), 3.38 (m, 2H), 3.04 (m, 1H), 2.86 (m, 1H), 2.27-2.16 (m, 1H), 1.90-1.67 (m, 1H). LCMS (ES, M+H=369).

Example 193 is made in a similar fashion to Example 192.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 193 | 4-[(3-hydroxypiperidin- | 374.49 | 375 | 9.08 (m, 1H), 8.85 (m, 1H), |
| | 4-yl)amino]-2-(3- | | | 8.50-8.10 (m, 2H), 8.46 (s, 1H), 7.91 (m, 1H), |
| | thienyl)thieno[3,2- | | | 7.75 (dd, 1H), 7.63 (br, 1H), 7.49 (dd, 1H), |
| | c]pyridine-7- | | | 5.83 (br, 1H), 4.33 (m, 1H), 3.95 (m, |
| | carboxamide | | | 1H), 3.40 (m, 2H), 3.01 (m, 1H), |
| | | | | 2.82 (m, 1H), 2.25-2.14 (m, 1H), |
| | | | | 1.96-1.78 (m, 1H) |

Examples 194-195 are made in a similar fashion to Example 58 using the appropriate starting material.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 194 | 4-{ethyl[(3S)-piperidin- | 380.51 | 381 | 9.26 (m, 1H), 8.95 (m, 1H), 8.63 (s, 1H), |
| | 3-yl]amino}-2- | | | 8.19 (m, 1H), 7.86 (m, 2H), 8.77 (s, 1H), |
| | phenylthieno[3,2- | | | 7.49 (m, 3H), 7.40 (m, 2H), 4.75 (m, 1H), |
| | c]pyridine-7- | | | 3.77 (m, 2H), 3.40 (m, 1H), 3.23 (m, 2H), |
| | carboxamide | | | 2.87 (m, 1H), 1.96 (m, 3H), 1.74 (m, 1H), |
| | | | | 1.17 (t, 3H). |
| 195 | 4-{ethyl[(3S)-piperidin- | 386.54 | 387 | 9.1 (m, 1H), 8.87 (m, 1H), 8.61 (s, 1H), |
| | 3-yl]amino}-2-(3- | | | 8.14 (m, 1H), 8.01 (s, 1H), 7.70 (m, 3H), |
| | thienyl)thieno[3,2- | | | 7.51 (m, 1H), 4.68 (m, 1H), 3.72 (m, 2H), |
| | c]pyridine-7- | | | 3.41 (m, 1H), 3.20 (m, 2H), 2.83 (m, 1H), |
| | carboxamide | | | 1.94 (m, 3H), 1.74 (m, 1H), 1.14 (t, 3H). |

Examples 196-197 are made in a similar fashion to Examples 69-70 using the appropriate starting materials.

| | | | MS | |
|---|---|---|---|---|
| | | MW | (ES, | ¹H NMR (300 MHz; d₆-DMSO; δ |
| Example | IUPAC Name | (g/mol) | M + H) | ppm) unless otherwise noted |
| 196 | 4-[(trans-2- | 380.51 | 381 | 9.05 (m, 1H), 8.86 (m, 1H), 8.50 (s, 1H), |
| | ethylpiperidin-3- | | | 8.43 (m, 1H), 8.18 (br s, 1H), 7.75 (d, |
| | yl)amino]-2- | | | 2H), 7.49 (d, 2H), 7.39 (dd, 1H), |
| | phenylthieno[3,2- | | | 7.34 (br s, 1H), 6.68 (br s, 1H), 4.48 (m, 1H), |
| | c]pyridine-7- | | | 3.26 (m, 2H), 2.97 (m, 1H), 2.05 (m, |
| | carboxamide | | | 1H), 1.88 (m, 3H), 1.63 (m, 2H), 0.95 (t, |
| | | | | 3H) |
| 197 | 4-[(cis-2- | 380.51 | 381 | 10.20 (m, 1H), 9.30 (m, 1H), 9.19 (m, |
| | ethylpiperidin-3- | | | 1H), 8.52 (s, 1H), 8.39 (br s, 1H), |
| | yl)amino]-2- | | | 7.82 (d, 2H), 7.67 (m, 1H), 7.50 (dd, 2H), |
| | phenylthieno[3,2- | | | 7.41 (dd, 1H), 6.75 (br s, 1H), 4.96 (m, |
| | c]pyridine-7- | | | 1H), 3.50 (m, 1H), 3.31 (m, 1H), |
| | carboxamide | | | 3.00 (m, 1H), 2.05 (m, 2H), 1.80 (m, 4H), |
| | | | | 0.86 (t, 3H) |

1. A compound of formula (I) 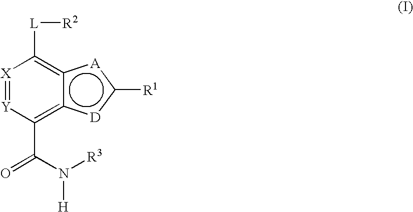
wherein:
A and D are each independently selected from N, CH, S, O and NR⁴;
L is selected from NR⁵, O and S;
X and Y are each independently selected from N and CH;
R¹ is selected from cyano, halo; C₁₋₆alkyl, —NR¹¹R¹², C₁₋₆alkoxy, C₂₋₆alkenyl, C₂₋₆alkynyl, cycloalkyl, cycloalkenyl, aryl, heterocyclyl, OR⁶; —COcarbocyclyl, —COheterocyclyl, —CO(C₁₋₆alkyl), —CONR²⁸, R²⁹, —S(O)ₓ(C₁₋₆alkyl), —S(O)ₓcarbocyclyl, —S(O)ₓheterocyclyl, S(O)_{y}NR²⁸R²⁹, and —(C₁₋₆alkyl)S(O)_{y}NR²⁸R²⁹ wherein x is independently 0 to 2 and y is independently 1 or 2; and wherein R¹ may be optionally substituted on one or more carbon atoms by one or more R⁹; and wherein if heterocyclyl contains an —NH— moiety, the nitrogen of said moiety may be optionally substituted by a group selected from R¹⁰;
R² is selected from (C₁₋₃alkyl)NR⁷R⁸, a 4- to 7-membered heterocyclyl ring containing at least one nitrogen atom, —COcarbocyclyl, —COheterocyclyl, —CO(C₁₋₆alkyl), —CONR²⁸R²⁹—CO₂(C₁₋₆alkyl), —CO₂-carbocyclyl, —CO₂heterocyclyl, —CO₂NR², R²⁹, —S(O)ₓ(C₁₋₆alkyl), —S(O)ₓcycloalkyl, —S(O)ₓcycloalkenyl, —S(O)ₓheterocyclyl, S(O)_{y}NR²⁸R²⁹, and —(C₁₋₆alkyl)S(O)_{y}NR²³R²⁹ wherein x is independently 0 to 2 and y is independently 1 or 2 and wherein R² may be optionally substituted on one or more carbon atoms by one or more R¹³; and further wherein if heterocyclyl contains an —NH— moiety, the nitrogen of said moiety may be optionally substituted by a group selected from R¹⁴;
R³ is selected from H, benzyl, C₁₋₆alkyl, cycloalkyl, cylcoalkenyl, aryl, heterocyclyl, OR⁶, CHO, —COcarbocyclyl, —CO(C₁₋₆alkyl), —CONR²⁸R²⁹, —S(O)ₓ(C₁₋₆alkyl), —S(O)ₓcarbocyclyl, —S(O)ₓheterocyclyl, S(O)_{y}NR²¹R²⁹, and —(C₁₋₆alkyl)S(O)_{y}NR²¹R²⁹ wherein x is independently 0 to 2, y is independently 1 or 2 and wherein R³ may be optionally substituted on one or more carbon atoms by one or more R¹⁵; and wherein if heterocyclyl contains a —NH— moiety, the nitrogen may be optionally substituted by a group selected from R¹⁶;
R⁴ is selected from H, C₁₋₃alkyl, cyclopropyl and CF₃;
R⁵ is selected from H, C₁₋₆alkyl, cycloalkyl, cylcoalkenyl, heterocyclyl and OR⁶; wherein R⁵ may be optionally substituted on carbon by one or more R¹⁷; and wherein if said heterocyclyl contains a —NH— moiety, the nitrogen of said moiety may be optionally substituted by a group selected from R¹⁸;
R⁶ is selected from H, C₁₋₆alkyl, cycloalkyl, cylcoalkenyl, aryl, and heterocyclyl; wherein R⁶ may be optionally substituted on carbon by one or more R¹⁹; and wherein if said heterocyclyl contains a —NH— moiety, the nitrogen of said moiety may be optionally substituted by a group selected from R²⁴;
R⁷ and R⁸ are independently selected from H, C₁₋₆alkyl, cycloalkyl, cylcoalkenyl, aryl, and heterocyclyl; wherein R⁷ and R⁸ independently of each other may be optionally substituted on carbon by one or more R²⁰; and wherein if said heterocyclyl contains a —NH— moiety, the nitrogen of said moiety may be optionally substituted by a group selected from R²¹.
R¹¹ and R¹² are independently selected from H, C₁₋₆alkyl, cycloalkyl, cylcoalkenyl, aryl, heterocyclyl, wherein R¹¹ and R¹² independently of each other may be optionally substituted on carbon by one or more R³²; and wherein if said heterocyclyl contains a —NH— moiety, the nitrogen of said moiety may be optionally substituted by a group selected from R³³;
R⁹, R¹³, R¹⁵, R¹⁷, R¹⁹, R²⁰, R³² and R³⁴ are each independently selected from halo, nitro, —NR²⁸R²⁹, cyano, isocyano, C₁₋₆alkyl, C₂₋₆alkenyl, C₂₋₆alkynyl, aryl, cycloalkyl, cylcoalkenyl, heterocyclyl, hydroxy, keto (═O), —O(C₁₋₆alkyl), —Ocarbocyclyl, —Oheterocyclyl, —Oaryl, —OC(O)C₁₋₆alkyl, —NHCHO, —N(C₁₋₆alkyl)CHO, —NHCONR²⁸R²⁹, —N(C₁₋₆alkyl)CONR²⁸R²⁹, —NHCO(C₁₋₆alkyl), —NHCOcarbocyclyl, —NHCO(heterocyclyl), —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR²⁸R²⁹, —CO(C₁₋₆alkyl), —COheterocyclyl, —COcycloalkyl, —COcycloalkenyl, —COaryl, —CO₂H, —CO₂(C₁₋₆alkyl), —CO₂-carbocyclyl, —CO₂heterocyclyl, —OC(O)(NR²⁸R²⁹), mercapto, —S(O)ₓ(C₁₋₆alkyl), —S(O)ₓcarbocyclyl, —S(O)ₓheterocyclyl, and —S(O)ₓNR²⁸R²⁹; wherein x is independently 0 to 2, wherein R⁹, R¹³, R¹⁵, R¹⁷, R¹⁹, R²⁰, R³² and R³⁴ independently of each other may be optionally substituted on carbon by one or more R²² and wherein if heterocyclyl contains a —NH— moiety, the nitrogen of said moiety may be optionally substituted by a group selected from R²³;
R¹⁰, R¹⁴, R¹⁶, R¹⁸, R²¹, R²⁴, R³³, and R³⁵ are each independently selected from cyano, C₁₋₆alkyl, C₂₋₆alkenyl, C₂₋₆alkynyl, aryl, cycloalkyl, cylcoalkenyl, heterocyclyl, hydroxy, —O(C₁₋₆alkyl), —Ocarbocyclyl, -amidino, —CHO, —CONR²⁸R²⁹, —CO(C₁₋₆alkyl), —COheterocyclyl, —COcarbocyclyl —COaryl, —CO₂(C₁₋₆alkyl), —CO₂-carbocyclyl, —CO₂heterocyclyl, —S(O)ₓ(C₁₋₆alkyl), —S(O)ₓcarbocyclyl, —S(O)ₓheterocyclyl, and —S(O)_{y}NR²⁸R²⁹; wherein x is independently 0 to 2, and y is independently 1 or 2; wherein R¹⁰, R¹⁴, R¹⁶, R¹⁸, R²¹, R²⁴, R³³ and R³⁵ independently of each other may be optionally substituted on carbon by one or more R²⁵ and wherein if said heterocyclyl contains a —NH— moiety, the nitrogen of said moiety may be optionally substituted by a group selected from R²⁶;
R²² and R²⁵ are each independently selected from halo, nitro, —NR²⁸R²⁹, cyano, isocyano, C₁₋₆alkyl, C₂₋₆alkenyl, C₂₋₆alkynyl, aryl, cycloalkyl, cylcoalkenyl, heterocyclyl, hydroxy, keto(═O), —O(C₁₋₆alkyl), —Ocarbocyclyl, —Oheterocyclyl, —Oaryl, —OC(O)C₁₋₆alkyl, —NHCHO, —N(C₁₋₆alkyl)CHO, —NHCONR²⁸R²⁹, —N(C₁₋₆alkyl)CONR²⁸R²⁹, —NHCO(C₁₋₆alkyl), —NHCOcarbocyclyl, —NHCO(heterocyclyl), —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR²⁸R²⁹, —CO(C₁₋₆alkyl), —COheterocyclyl, —COcycloalkyl, —COcycloalkenyl, —CO₂H, —CO₂(C₁₋₆alkyl), —CO₂-carbocyclyl, —OC(O)(NR²⁸R²⁹), mercapto, —S(O)ₓ(C₁₋₆alkyl), —S(O)ₓcarbocyclyl, —S(O)ₓheterocyclyl, and —S(O)ₓNR²⁸R²⁹; wherein x is independently 0 to 2, wherein R²² and R²⁵ may be optionally substituted on carbon by one or more R³⁶ and wherein if said heterocyclyl contains a —NH— moiety, the nitrogen of said moiety may be optionally substituted by a group selected from R²⁷.
R²³ and R²⁶ are each independently selected from cyano, C₁₋₆alkyl, C₂₋₆alkenyl, C₂₋₆alkynyl, aryl, cycloalkyl, cylcoalkenyl, heterocyclyl, hydroxy, —O(C₁₋₆alkyl), —Ocarbocyclyl, -amidino, —CHO, —CONR²⁸R²⁹, —CO(C₁₋₆alkyl), —COheterocyclyl, —COcycloalkyl, —COcycloalkenyl, —CO₂(C₁₋₆alkyl), —CO₂-carbocyclyl, —S(O)ₓ(C₁₋₆alkyl), —S(O)ₓcarbocyclyl, —S(O)ₓheterocyclyl, and —S(O)_{y}NR²⁸R²⁹; wherein x is independently 0 to 2, and y is independently 1 or 2; wherein R²³ and R²⁶ independently of each other may be optionally substituted on carbon by one or more R³⁰ and wherein if said heterocyclyl contains a —NH— moiety, the nitrogen of said moiety may be optionally substituted by a group selected from R³¹.
R²⁸ and R²⁹ are each independently selected from H, amino, cyano, C₁₋₆alkyl, C₂₋₆alkenyl, C₂₋₆alkynyl, aryl, cycloalkyl, cycloalkenyl, heterocyclyl, hydroxy, —O(C₁₋₆alkyl), —Oaryl, —OCOalkyl, -amidino, —CHO, —CO(C₁₋₆alkyl), —COheterocyclyl, —COcycloalkyl, —COcycloalkenyl, —SO(C₁₋₆alkyl), —SO₂(C₁₋₆alkyl), wherein R²⁸ and R²⁹ independently of each other may be optionally substituted on carbon by one or more R³⁴; and wherein if said heterocyclyl contains a —NH— the nitrogen of said moiety may be optionally substituted by a group selected from R³⁵;
R³⁰ and R³⁶ are each independently selected from halo, nitro, —NR², R²⁹, cyano, isocyano, C₁₋₆alkyl, C₂₋₆alkenyl, C₂₋₆alkynyl, aryl, cycloalkyl, cylcoalkenyl, heterocyclyl, hydroxy, keto (═O), —O(C₁₋₆alkyl), —Ocarbocyclyl, —OC(O)C₁₋₆alkyl, —NHCHO, —N(C₁₋₆alkyl)CHO, —NHCONR²⁸R²⁹, —N(C₁₋₆alkyl)CONR²⁸R²⁹, —NHCO(C₁₋₆alkyl), —NHCOcarbocyclyl, —NHCO(heterocyclyl), —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR²⁸R²⁹, —CO(C₁₋₆alkyl), —COheterocyclyl, —COcycloalkyl, —COcycloalkenyl, —CO₂H, —CO₂(C₁₋₆alkyl), —CO₂-carbocyclyl, —OC(O)(NR²³R²⁹), mercapto, —S(O)ₓ(C₁₋₆alkyl), —S(O)ₓcarbocyclyl, —S(O)ₓheterocyclyl, and —S(O)ₓNR²³R²⁹; wherein x is independently 0 to 2;
R²⁷ and R³¹ are each independently selected from cyano, C₁₋₆alkyl, C₂₋₆alkenyl, C₂₋₆alkynyl, aryl, cycloalkyl, cylcoalkenyl, heterocyclyl, hydroxy, —O(C₁₋₆alkyl), —Ocarbocyclyl, —(C₁₋₆alkyl)-O—(C₁₋₆alkyl), -amidino, —CHO, —CONR²⁸R²⁹, —CO(C₁₋₆alkyl), —COheterocyclyl, —COcycloalkyl, —COcycloalkenyl, —CO₂(C₁₋₆alkyl), —CO₂-carbocyclyl, —S(O)ₓ(C₁₋₆alkyl), —S(O)ₓcarbocyclyl, —S(O)ₓheterocyclyl, and —S(O)_{y}NR²⁸R²⁹; wherein x is independently 0 to 2, and y is independently 1 or 2;
or a pharmaceutically acceptable salt thereof.

2. A compound, or a pharmaceutically acceptable salt thereof, according to claim 1 wherein R² is selected from (C₁₋₃alkyl)NR⁷R⁸ and a 4- to 7-membered heterocyclyl ring containing at least one nitrogen atom wherein R² may be optionally substituted on one or more carbon atoms by one or more R¹³; and further wherein if heterocyclyl contains an —NH— moiety, the nitrogen of said moiety may be optionally substituted by a group selected from R¹⁴.

3. A compound, or a pharmaceutically acceptable salt thereof, according to claim 1 wherein R² is a 4- to 7-membered heterocyclyl ring containing at least one nitrogen atom wherein R² may be optionally substituted on one or more carbon atoms by one or more R¹³; and further wherein if heterocyclyl contains an —NH— moiety, the nitrogen of said moiety may be optionally substituted by a group selected from R¹⁴.

4. A compound, or a pharmaceutically acceptable salt thereof, according to claim 1 wherein R¹ is selected from aryl and heterocyclyl and wherein R¹ may be optionally substituted on one or more carbon atoms by one or more R⁹; and wherein if heterocyclyl contains an —NH— moiety, the nitrogen of said moiety may be optionally substituted by a group selected from R¹⁰.

5. A compound, or a pharmaceutically acceptable salt thereof, according to claim 1 wherein R³ is H.

6. A compound, or a pharmaceutically acceptable salt thereof, according to claim 1 wherein X is N; Y is CH; A is CH and D is S.

7. A compound, or a pharmaceutically acceptable salt thereof, according to claim 1 wherein X is CH; Y is CH; A is CH and D is NR⁴.

8. A compound, or a pharmaceutically acceptable salt thereof, according to claim 1 wherein L is NR⁵.

9. A compound of formula I, according to claim 1, wherein
A is CH;
D is S;
L is NR⁵
X is N;
Y is CH;
R¹ is selected from C₁₋₆alkyl, aryl and heterocyclyl wherein R¹ may be optionally substituted on one or more carbon atoms by one or more R⁹; and wherein if heterocyclyl contains an —NH— moiety, the nitrogen of said moiety may be optionally substituted by a group selected from R¹⁰;
R² is a 4- to 7-membered heterocyclyl ring containing at least one nitrogen atom, wherein R² may be optionally substituted on one or more carbon atoms by one or more R¹³; and further wherein if heterocyclyl contains an —NH— moiety, the nitrogen of said moiety may be optionally substituted by a group selected from R¹⁴;
R³ is H;
R⁵ is H or C₁₋₃alkyl; or a pharmaceutically acceptable salt thereof.

10. A compound of formula I, according to claim 1, wherein
A is CH;
D is NR⁴
L is NR⁵;
X is CH;
Y is CH;
R¹ is selected from C₁₋₆alkyl, aryl and heterocyclyl wherein R¹ may be optionally substituted on one or more carbon atoms by one or more R⁹; and wherein if heterocyclyl contains an —NH— moiety, the nitrogen of said moiety may be optionally substituted by a group selected from R¹⁰;
R² is a 4- to 7-membered heterocyclyl ring containing at least one nitrogen atom, wherein R² may be optionally substituted on one or more carbon atoms by one or more R¹³; and further wherein if heterocyclyl contains an —NH— moiety, the nitrogen of said moiety may be optionally substituted by a group selected from R¹⁴;
R³ is H;
R⁴ is H, C₁₋₃alkyl, cyclopropyl and CF₃;
R⁵ is H or C₁₋₃alkyl; or a pharmaceutically acceptable salt thereof.

11. A compound of formula I according to claim 1 or a pharmaceutically acceptable salt thereof selected from
2-phenyl-4-[(3S)-piperidin-3-ylamino]thieno[3,2-c]pyridine-7-carboxamide;
4-[(3S)-piperidin-3-ylamino]-2-(3-thienyl)thieno[3,2-c]pyridine-7-carboxamide;
2-(3-fluorophenyl)-4-[(3S)-piperidin-3-ylamino]thieno[3,2-c]pyridine-7-carboxamide;
4-[(3S)-piperidin-3-ylamino]-2-(2-thienyl)thieno[3,2-c]pyridine-7-carboxamide;
2-(4-fluorophenyl)-4-[(3S)-piperidin-3-ylamino]thieno[3,2-c]pyridine-7-carboxamide;
2-(3,4-difluorophenyl)-4-[(3S)-piperidin-3-ylamino]thieno[3,2-c]pyridine-7-carboxamide;
2-(1-benzyl-1H-pyrazol-4-yl)-4-[(3S)-piperidin-3-ylamino]thieno[3,2-c]pyridine-7-carboxamide;
4-{methyl[(3S)-piperidin-3-yl]amino}-2-phenylthieno[3,2-c]pyridine-7-carboxamide;
2-(3-fluorophenyl)-4-{methyl [(3S)-piperidin-3-yl]amino}thieno[3,2-c]pyridine-7-carboxamide;
2-(4-fluorophenyl)-4-{methyl [(3S)-piperidin-3-yl]amino}thieno[3,2-c]pyridine-7-carboxamide;
4-{methyl[(3S)-piperidin-3-yl]amino}-2-(3-thienyl)thieno[3,2-c]pyridine-7-carboxamide;
4-{[trans-2-methylpiperidin-3-yl]amino}-2-phenylthieno[3,2-c]pyridine-7-carboxamide;
2-(3-fluorophenyl)-4-{[trans-2-methylpiperidin-3-yl]amino}thieno[3,2-c]pyridine-7-carboxamide;
4-{[trans-2-methylpiperidin-3-yl]amino}-2-(3-thienyl)thieno[3,2-c]pyridine-7-carboxamide;
2-(4-fluorophenyl)-4-{[trans-2-methylpiperidin-3-yl]amino}thieno[3,2-c]pyridine-7-carboxamide;
4-{[(2R,3S)-2-methylpiperidin-3-yl]amino}-2-phenylthieno[3,2-c]pyridine-7-carboxamide;
4-{[(2R,3S)-2-methylpiperidin-3-yl]amino}-2-(3-thienyl)thieno[3,2-c]pyridine-7-carboxamide;
4-{methyl[trans-2-methylpiperidin-3-yl]amino}-2-phenylthieno[3,2-c]pyridine-7-carboxamide;
4-[(2,6-dimethylpiperidin-3-yl)amino]-2-phenylthieno[3,2-c]pyridine-7-carboxamide;
4-[(2,6-dimethylpiperidin-3-yl)amino]-2-(3-fluorophenyl)thieno[3,2-c]pyridine-7-carboxamide;
4-[(2,6-dimethylpiperidin-3-yl)amino]-2-(3-thienyl)thieno[3,2-c]pyridine-7-carboxamide;
4-[(6-methylpiperidin-3-yl)amino]-2-(3-thienyl)thieno[3,2-c]pyridine-7-carboxamide;
4-[(6-methylpiperidin-3-yl)amino]-2-phenylthieno[3,2-c]pyridine-7-carboxamide;
2-{4-[(dimethylamino)methyl]phenyl}-4-[(3S)-piperidin-3-ylamino]thieno[3,2-c]pyridine-7-carboxamide;
4-[(3S)-piperidin-3-ylamino]-2-[4-(piperidin-1-ylmethyl)phenyl]thieno[3,2-c]pyridine-7-carboxamide;
2-[4-(morpholin-4-ylmethyl)phenyl]-4-[(3S)-piperidin-3-ylamino]thieno[3,2-c]pyridine-7-carboxamide;
2-(4-chlorophenyl)-4-(piperidin-3-ylamino)-1H-indole-7-carboxamide;
2-(4-fluorophenyl)-4-(piperidin-3-ylamino)-1H-indole-7-carboxamide;
2-phenyl-4-[(3S)-piperidin-3-ylamino]-1H-indole-7-carboxamide;
2-(3-fluorophenyl)-4-[(3S)-piperidin-3-ylamino]-1H-indole-7-carboxamide;
2-(4-chlorophenyl)-4-[(3S)-piperidin-3-ylamino]-1H-indole-7-carboxamide;
2-(4-fluorophenyl)-4-[(3S)-piperidin-3-ylamino]-1H-indole-7-carboxamide;
4-{ethyl [(3S)-piperidin-3-yl]amino}-2-phenylthieno[3,2-c]pyridine-7-carboxamide;
4-{ethyl [(3S)-piperidin-3-yl]amino}-2-(3-thienyl)thieno[3,2-c]pyridine-7-carboxamide;
4-[(trans-2-ethylpiperidin-3-yl)amino]-2-phenylthieno[3,2-c]pyridine-7-carboxamide;
and 4-[(cis-2-ethylpiperidin-3-yl)amino]-2-phenylthieno[3,2-c]pyridine-7-carboxamide.

12. (canceled)

13. (canceled)

14. (canceled)

15. (canceled)

16. A method of treatment of a human or animal suffering from cancer comprising administering to said human or animal a therapeutically effective amount of a compound of formula (I) or a pharmaceutically acceptable salt thereof, as claimed in claim 1.

17. (canceled)

18. A method of treatment of a human or animal suffering from a neoplastic disease such as carcinoma of the breast, ovary, colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system, and other tumor types such as melanoma, multiple myeloma, fibrosarcoma and osteosarcoma, and malignant brain tumors;
or a pharmaceutically acceptable salt thereof, as claimed in claim 1.

19. (canceled)

20. (canceled)

21. (canceled)

22. (canceled)

23. (canceled)

24. A pharmaceutical composition comprising a compound of formula (I) or a pharmaceutically acceptable salt thereof, as claimed in claim 1, together with at least one pharmaceutically acceptable carrier, diluent or excipient.

25. (canceled)

26. (canceled)

27. (canceled)

28. (canceled)

29. A method of inhibiting CHK1 kinase comprising administering to an animal or human in need of said inhibiting a therapeutically effective amount of a compound of formula (I) or a pharmaceutically acceptable salt thereof, as claimed in claim 1.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. A method of treatment according to claim 16 additionally comprising administering to said human or animal a therapeutically effective amount of an anti-tumor agent.

42. A method of treatment according to claim 16 additionally comprising administering to said human or animal a therapeutically effective amount of a DNA damaging agent.
